(19) United States
(12) Patent Application Publication
(10) Pub. No.: US 2009/0264447 A1
(43) Pub. Date: Oct. 22, 2009

(54) Pyrimidine compounds for combating pathogenic fungi and cancer

(75) Inventors: Jochen Dietz, Mannheim (DE); Bernd Müller, Frankenthal (DE); Jan Klaas Lohmann, Ludwigshafen (DE); Jens Renner, Bad Dürkheim (DE); Sarah Ulmschneider, Bad Durkheim (DE); Marianna Vrettou, Mannheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/375,763

(22) PCT Filed: Aug. 1, 2007
(86) PCT No.: PCT/EP2007/057989

§ 371 Date: Jan. 30, 2009

(30) Foreign Application Priority Data
Aug. 2, 2006 (EP) 06118350.5

(51) Int. Cl. A01N 43/54; C07D 239/42; A61K 31/505; C07D 403/04; C07D 403/12; A61P 35/00

(52) U.S. Cl. 514/256; 544/326; 544/328; 544/296

(57) ABSTRACT

The present invention relates to the use of pyrimidine compounds of formula I

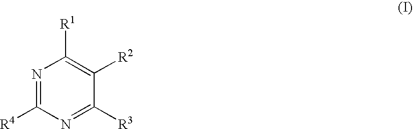

wherein the variables have the meanings stated in the claims and in the description, for combating pathogenic fungi, new pyrimidine compounds of formula (I), and fungicidal and pharmaceutical agents containing the same.

The present invention relates to the use of 5-(het)arylpyrimidines for controlling harmful fungi, to novel 5-(het)arylpyrimidines and to fungicidal or pharmaceutical compositions comprising at least one such compound as active component.

Fungicidally active 5-phenyl- and 5-hetarylpyrimidines which carry an amino group, a (thio)ether group or an aliphatic, carbocyclic or heterocyclic radical attached via carbon in the 6-position are generally known and described, for example, in WO 01/96314, WO 03/043993, WO 03/070721, WO 2004/087678, WO 2004/103978, WO 2005/012261, WO 2005/019187 and WO 2005/070899.

WO 2005/030216 describes 5-phenylpyrimidines which carry a hydroxyalkoxy, aminoalkoxy, hydroxyalkylthio, aminoalkylthio, hydroxyalkylamino or aminoalkylamino group on the phenyl ring, which are substituted in the 6-position by a secondary amino group or a cycloalkyl group and which carry an amino group, a cyanamide group, an aryl or a hetaryl substituent in the 2-position. These compounds are said to be suitable for the treatment of cancer. An application in crop protection is not mentioned.

The pyrimidine compounds known as fungicides from the prior art are, with a view to their fungicidal activity, sometimes unsatisfactory, or they have unwanted properties, such as low crop plant compatibility.

Accordingly, it is an object of the present invention to provide compounds having better fungicidal activity and/or better crop plant compatibility.

Moreover, it was an object to provide novel pyrimidine compounds having, compared to the pyrimidines of the prior art, improved pharmacological action.

Surprisingly, these objects are achieved by pyrimidine compounds of the general formula I, defined below, and by the agriculturally acceptable salts of the compounds I.

Accordingly, the present invention relates to the use of pyrimidine compounds of the formula I

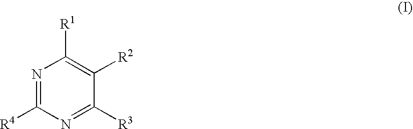

in which
R¹ is C₁-C₁₀-alkyl, C₂-C₁₀-alkenyl, C₂-C₁₀-alkynyl, C₃-C₁₀-cycloalkyl, C₃-C₁₀-cycloalkenyl, phenyl, naphthyl or a saturated or unsaturated aromatic or non-aromatic 5-, 6-, 7-, 8-, 9- or 10-membered heterocycle, where the heterocycle contains 1, 2, 3 or 4 heteroatoms selected from the group consisting of O, S and N as ring members and may furthermore contain one or two CO groups as ring members, where R¹ may be partially or fully halogenated and/or may carry 1, 2, 3 or 4 identical or different substituents L³; or
   is a radical of the formula NR⁵R⁶, OR⁷ or SR⁸;
R² is phenyl or a 5- or 6-membered heteroaromatic radical, where the heteroaromatic radical contains 1, 2, 3 or 4 heteroatoms selected from the group consisting of O, S and N as ring members, where phenyl or the heteroaromatic radical carries a substituent L¹ and optionally 1, 2, 3 or 4 identical or different substituents L²;
R³ is halogen, hydroxyl, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-alkylsulfonyl, C₁-C₄-alkoxy-C₁-C₄-alkyl, cyano-C₁-C₄-alkyl or cyano;
R⁴ is halogen, cyano, hydroxyl, mercapto, N₃, C₁-C₆-alkyl, C₂-C₈-alkenyl, C₂-C₈-alkynyl, C₁-C₆-alkoxy, C₃-C₈-alkenyloxy, C₃-C₈-alkynyloxy, C₁-C₆-alkylthio, C₃-C₈-alkenylthio, C₃-C₈-alkynylthio, C₁-C₆-alkylsulfinyl, C₁-C₆-alkylsulfonyl, hydroxysulfonyl, aminosulfonyl, C₁-C₆-alkylaminosulfonyl, di-C₁-C₆-alkylaminosulfonyl,
   C₃-C₁₀-cycloalkyl, phenyl, naphthyl, 3-, 4-, 5-, 6-, 7-, 8-, 9- or 10-membered saturated, partially unsaturated or aromatic heterocyclyl having 1, 2, 3 or 4 heteroatoms selected from the group consisting of O, N and S and optionally 1 or 2 carbonyl groups as ring members,
   or a radical of the formula —ON(═CR^{a}R^{b}), —NR^{c}N═CR^{a}R^{b}, —NR^{a}R^{b}, —NR^{c}NR^{a}R^{b}, —NR^{a}—CN, —N═OR^{a}; —NR^{c}C(═W)—NR^{a}R^{b}, —NR^{a}C(═W)R^{c}, —NNR^{a}R^{b}C(═W)—X¹—R^{c}, —OC(═W)R^{c}, —O(C═W)NR^{a}R^{b}, —C(═W)R^{c}, —C(═W)NR^{a}R^{b}, —C(═W)NR^{a}OR^{b}, —CR^{a}R^{b}—OR^{c}, —CR^{a}R^{b}—SR^{c}, —CR^{a}R^{b}—NR^{c}R^{d}, —CR^{a}R^{b}—C(═W)R^{c}, —C(═W)—NR^{a}—X²—R^{b}, —C(═NX²R^{a})—OR^{b} or —C(═NX²R^{a})—SR^{b},
   in which
   W is O, S, NR^{d} or NNR^{d}R^{e};
   X¹ is O or NR^{f};
   X² is a single bond, —CO—, —CONH—, —COO—, —O—, —NR^{f}—, —CH₂—O—CO— or —CH═CH—(C═O)—, where the left part of the divalent radicals is attached to the nitrogen atom;
   R^{a}, R^{b}, R^{c}, R^{d}, R^{e}, R^{f} independently of one another are hydrogen, hydroxyl, C₁-C₆-alkyl, C₂-C₈-alkenyl, C₂-C₈-alkynyl, C₁-C₆-alkoxy, C₁-C₄-alkoxy-C₁-C₄-alkyl, C₁-C₆-alkylcarbonyl, C₃-C₆-cycloalkyl, C₃-C₆-cycloalkenyl, C₃-C₆-cycloalkoxy, aryl, aryl-C₁-C₄-alkyl or 5-, 6-, 7-, 8-, 9- or 10-membered heterocyclyl having 1, 2, 3 or 4 heteroatoms selected from the group consisting of O, N and S and optionally 1 or 2 carbonyl groups as ring members;
      where, if R^{a}, R^{b}, R^{c} are attached directly to an oxygen atom, they are not hydroxyl, C₁-C₆-alkoxy or C₃-C₆-cycloalkoxy;
      or R^{a} and R^{b} together with the nitrogen atom to which they are attached form a group R^{c}—X¹¹—C(R^{g})═N in which
      R^{g} is independently defined like R^{a} or as halogen or cyano; and
      X¹¹ is independently defined like X¹;
      or two of the radicals R^{a}, R^{b}, R^{c}, R^{d}, R^{e}, R^{f}, R^{g} together form a C₂-C₄-alkylene group which may be interrupted by an oxygen atom and/or may contain a C—C double bond,
   where the aliphatic, alicyclic, aromatic and/or heterocyclic groups in R⁴, R^{a}, R^{b}, R^{c}, R^{d}, R^{e}, R^{f} and/or R^{g} may be partially or fully halogenated and/or may have 1, 2 or 3 substituents R^{x}, where
   R^{x} is cyano, nitro, amino, aminocarbonyl, aminothiocarbonyl, hydroxyl, mercapto, oxo, carboxyl, C₁-C₆-alkyl, C₁-C₆-haloalkyl, C₁-C₆-alkylcarbonyl, C₃-C₆-cycloalkyl, C₃-C₆-halocycloalkyl, C₃-C₆-cycloalkyl-C₁-C₄-alkyl, C₃-C₆-cycloalkenyl, C₁-C₆-alkoxy, C₁-C₆-haloalkoxy, C₁-C₆-alkyloxycarbonyl, C₁-C₆-alkylthio, C₁-C₆-alkylsulfinyl, C₁-C₆-alkylsulfonyl, hydroxysulfonyl, aminosulfonyl, C₁-C₆-alkylaminosulfonyl, di-C₁-C₆-alkylaminosulfonyl, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₁-C₆-alkylcarbonylamino, C₂-C₆-alkenyl, C₂-C₆-alkynyl, C₂-C₆-alkenyloxy, C₃-C₆-alkynyloxy, tri-C₁-C₆-alkylsilyl, aryl, aryloxy, aryl-C₁-C₄-alkyl, aryl-C₁-C₄-alkoxy, 5- or 6-membered saturated, partially unsaturated or aromatic heterocyclyl, 5- or 6-membered saturated, partially unsaturated or aromatic heterocyclyloxy, 5- or 6-membered saturated, partially unsaturated or aromatic heterocyclylcarbonyl, where the heterocyclyl radicals in the three last-mentioned groups contain 1, 2, 3 or 4 heteroatoms selected from the group consisting of O, N and S and optionally 1 or 2 carbonyl groups as ring members, —C(═NOR^{α})—OR^{β} or —OC(R^{α})₂—C(R^{β})═NOR^{β},
   where the cyclic radicals in R^{x} may be unsubstituted or may carry 1, 2 or 3 radicals R^{y}, where
   R^{y} is cyano, nitro, halogen, hydroxyl, amino, aminocarbonyl, aminothiocarbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl,
      C₃-C₆-cycloalkenyl, phenyl, phenoxy, phenylthio, benzyl, benzyloxy, 5- or 6-membered saturated, partially unsaturated or aromatic heterocyclyl, 5- or 6-membered saturated, partially unsaturated or aromatic heterocyclyloxy, where the heterocyclyl radicals in the two last-mentioned groups contain 1, 2, 3 or 4 heteroatoms selected from the group consisting of O, N and S and optionally 1 or 2 carbonyl groups as ring members, or —C(═NOR^{α})—OR^{β};
   R^{α}, R^{β} independently of one another are hydrogen or C₁-C₆-alkyl;
R⁵ is H, C₁-C₁₀-alkyl, C₂-C₁₀-hydroxyalkyl, C₂-C₁₀-alkenyl, C₂-C₁₀-alkynyl, C₄-C₁₀-alkadienyl, C₃-C₁₀-cycloalkyl, C₁-C₁₀-alkoxy, C₂-C₁₀-alkenyloxy, C₂-C₁₀-alkynyloxy, amino, C₁-C₈-alkylamino, di-(C₁-C₈-alkyl)amino, phenyl, naphthyl or a saturated or unsaturated aromatic or non-aromatic 5- or 6-membered heterocycle which is attached via a carbon atom, where the heterocycle contains 1, 2, 3 or 4 heteroatoms selected from the group consisting of O, S and N as ring members and may furthermore contain 1 or 2 CO groups as ring members;
   where the aliphatic, alicyclic, aromatic and/or heterocyclic groups in R⁵ may be partially or fully halogenated and/or may carry 1, 2, 3 or 4 identical or different substituents R^{a1};
R⁶ is independently defined like R⁵, with the proviso that R⁵ and R⁶ are not both H, or is a group #-CR⁶¹R⁶²—(CR⁶³R⁶⁴)_{q}—(CR⁶⁵R⁶⁶)ₚ—Y-Z in which
   # is the point of attachment to the nitrogen atom;
   R⁶¹, R⁶², R⁶³, R⁶⁴, R⁶⁵ and R⁶⁶ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle which contains one, two, three or four heteroatoms from the group consisting of O, N and S; where
      R⁶³ with R⁶¹ or R⁶⁶ together with the atoms to which these radicals are attached may also form a five-, six-, seven-, eight-, nine- or ten-membered saturated or partially unsaturated ring which, in addition to carbon atoms, may contain one, two or three heteroatoms from the group consisting of O, N and S as ring members and/or may carry one or more substituents R^{a1};
      R⁶¹ with R⁶², R⁶³ with R⁶⁴, R⁶⁵ with R⁶⁶ in each case together may also be oxygen, thus forming carbonyl groups, and form a C₂-C₅-alkylene, C₂-C₅-alkenylene or C₂-C₅-alkynylene chain (which may be interrupted by one, two or three heteroatoms from the group consisting of O, N and S), thus forming spiro groups;
      R⁵ and R⁶¹ together with atoms to which they are attached may form a 5-, 6-, 7-, 8-, 9- or 10-membered saturated or partially unsaturated heterocycle which, in addition to carbon atoms, may contain one, two or three further heteroatoms from the group consisting of O, N and S as ring members;
   where
   the aliphatic, alicyclic, heterocyclic, aromatic and/or heteroaromatic radicals in R⁶¹ to R⁶⁶ in each case independently of one another may be partially or fully halogenated and/or may carry one, two, three or four identical or different groups R^{a1};
   each R^{a1} is independently cyano, nitro, hydroxyl, carboxyl, C₁-C₆-alkyl, C₂-C₈-alkenyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C₁-C₆-alkylthio, amino, C₁-C₆-alkylamino, di-(C₁-C₆-alkyl)amino, C(O)R^{π}, C(S)R^{π}, C(O)OR^{π}, C(S)OR^{π}, C(O)SR^{π}, C(S)SR^{π}, C(O)NH₂, C(O)NHR^{π}, C(O)NR^{π}₂, OC(O)OR^{π}, OC(O)NH₂, OC(O)NHR^{π}, OC(O)NR^{π}₂, C₁-C₆-alkylene, oxy-C₁-C₄-alkylene, oxy-C₁-C₃-alkyleneoxy, where the three last-mentioned divalent groups may be attached to the same atom or to adjacent atoms, phenyl, naphthyl or a 5-, 6-, 7-, 8-, 9- or 10-membered saturated, partially unsaturated or aromatic heterocycle which contains one, two, three or four heteroatoms from the group consisting of O, N and S;
   each R^{π} is independently C₁-C₈-alkyl, C₃-C₈-alkenyl, C₃-C₈-alkynyl, C₃-C₆-cycloalkyl or C₃-C₆-cycloalkenyl;
   where the aliphatic, alicyclic, aromatic or heterocyclic groups in the abovementioned groups R^{a1} and R^{π} for their part may be partially or fully halogenated and/or may carry one, two or three groups R^{b1};
   each R^{b1} is independently cyano, nitro, hydroxyl, mercapto, amino, carboxyl, C₁-C₆-alkyl, C₂-C₈-alkenyl, C₁-C₆-alkoxy, C₂-C₈-alkenyloxy, C₂-C₈-alkynyloxy, C₁-C₆-alkylthio, C₁-C₆-alkylamino, di-(C₁-C₆-alkyl)amino, formyl, C₁-C₆-alkylcarbonyl, C₁-C₆-alkylsulfonyl, C₁-C₆-alkylsulfinyl, C₁-C₆-alkoxycarbonyl, C₁-C₆-alkylcarbonyloxy, C₁-C₆-alkoxycarbonyloxy, aminocarbonyl, aminothiocarbonyl, C₁-C₆-alkylaminocarbonyl, di-(C₁-C₆-alkyl)aminocarbonyl, C₁-C₆-alkylaminothiocarbonyl, di-(C₁-C₆-alkyl)aminothiocarbonyl, C₃-C₁₀-cycloalkyl, C₃-C₁₀-cycloalkoxy, heterocyclyl, heterocyclyloxy, where heterocyclyl in the two last-mentioned radicals is 3-, 4-, 5-, 6-, 7-, 8-, 9- or 10-membered and contains 1, 2, 3 or 4 heteroatoms selected from the group consisting of O, N and S and optionally 1 or 2 carbonyl groups as ring members; aryl, aryloxy, arylthio, aryl-C₁-C₆-alkoxy, aryl-C₁-C₆-alkyl, hetaryl, hetaryloxy or hetarylthio, where the aryl radicals contain 6 to 10 ring members and the hetaryl radicals 5 or 6 ring members and 1, 2, 3 or 4 heteroatoms selected from the group consisting of O, N and S, where the alicyclic, heterocyclic, aromatic and/or heteroaromatic systems may be partially or fully halogenated and/or substituted by 1, 2, 3, 4 or 5 C₁-C₄-alkyl and/or C₁-C₄-haloalkyl groups;
   p is 0, 1, 2, 3, 4 or 5;
   q is 0 or 1;
   Y is oxygen or sulfur;
   Z is hydrogen, carboxyl, formyl, C₁-C₈-alkyl, C₂-C₈-alkenyl, C₂-C₈-alkynyl, C₃-C₆-cycloalkyl, C₃-C₈-cycloalkenyl, C(O)R^{π}, C(O)OR^{π}, C(S)OR^{π}, C(O)SR^{π}, C(S)SR^{π}, C(NR^{A})SR^{π}, C(S)R^{π}, C(NR^{π})NR^{A}R^{B}, C(NR^{π})R^{A}, C(NR^{π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkylene-NR^{A}C(NR^{π})NR^{A}R^{B}, C(S)—C₁-C₄-alkylene-NR^{A}C(NR^{π})NR^{A}R^{B}, C(NR^{π})—C₁-C₄-alkylene-NR^{A}C(NR^{π})NR^{A}R^{B}, phenyl, naphthyl, a five-, six-, seven-, eight-, nine- or ten-membered saturated, partially unsaturated or aromatic heterocycle which contains one, two, three or four heteroatoms from the group consisting of O, N and S and which is attached directly or via a carbonyl, thiocarbonyl, C₁-C₄-alkylcarbonyl or C₁-C₄-alkylthiocarbonyl group; where the carbon chains in group Z may be substituted by one or more groups R^{b1};
   R^{A} and R^{B} independently of one another are hydrogen, C₂-alkenyl, C₂-alkynyl or one of the groups mentioned under R^{π}; or
      R^{A} and R^{B} together with the nitrogen atom to which they are attached or R^{A} and R^{π} together with the carbon atoms and heteroatoms to which they are attached may also form a five- or six-membered saturated, partially unsaturated or aromatic ring which, in addition to carbon atoms, may contain one, two or three further heteroatoms from the group consisting of O, N and S as ring members and/or may carry one or more substituents R^{a1};
   or
   Z with R⁶⁴ or R⁶⁶ may also form a five- or six-membered saturated or partially unsaturated ring which, in addition to carbon atoms and Y, may contain one or two further heteroatoms from the group consisting of O, N and S as ring members and/or may carry one or more substituents R^{a1};
   where the group Z may be partially or fully halogenated and/or carry one, two or three groups R^{b1};
   or R⁵ and R⁶ together with the nitrogen atom to which they are attached form a saturated or unsaturated aromatic or non-aromatic 5-, 6-, 7- or 8-membered heterocycle, where the heterocycle may additionally contain 1, 2 or 3 heteroatoms selected from the group consisting of O, S and N and/or 1 or 2 CO groups as ring members and where the heterocycle may carry 1, 2 or 3 substituents selected from the group consisting of halogen, hydroxyl, cyano, nitro, carboxyl, C₁-C₈-alkyl, C₁-C₈-haloalkyl, C₂-C₈-hydroxyalkyl, C₁-C₈-alkoxy, C₁-C₈-haloalkoxy, C₁-C₈-alkylthio, C₁-C₈-haloalkylthio, C₂-C₈-alkenyl, C₂-C₈-haloalkenyl, C₂-C₈-alkenyloxy, C₂-C₈-haloalkenyloxy, C₂-C₈-alkynyl, C₃-C₈-haloalkynyl, C₂-C₈-alkynyloxy, C₃-C₈-haloalkynyloxy, C₃-C₈-cycloalkyl, C₃-C₈-cycloalkoxy, C₃-C₈-cycloalkenyl, C₃-C₈-cycloalkenyloxy, amino, C₁-C₈-alkylamino, di-(C₁-C₈-alkyl)amino, C₁-C₈-alkylcarbonyl, C₁-C₈-haloalkylcarbonyl, C₂-C₈-alkenylcarbonyl, C₂-C₈-haloalkenylcarbonyl, C₂-C₈-alkynylcarbonyl, C₃-C₈-haloalkynylcarbonyl, C₃-C₈-cycloalkylcarbonyl, C₃-C₈-cycloalkenylcarbonyl, C₁-C₈-alkylcarbonyloxy, C₁-C₈-haloalkylcarbonyloxy, C₂-C₈-alkenylcarbonyloxy, C₂-C₈-haloalkenylcarbonyloxy, C₂-C₈-alkynylcarbonyloxy, C₃-C₈-haloalkynylcarbonyloxy, C₃-C₈-cycloalkylcarbonyloxy, C₃-C₈-cycloalkenylcarbonyloxy, C₁-C₈-alkoxycarbonyl, C₁-C₈-haloalkoxycarbonyl, C₂-C₈-alkenyloxycarbonyl, C₂-C₈-haloalkenyloxycarbonyl, C₂-C₈-alkynyloxycarbonyl, C₃-C₈-haloalkynyloxycarbonyl, C₃-C₈-cycloalkoxycarbonyl, cycloalkenyloxycarbonyl, aminocarbonyl, C₁-C₈-alkylaminocarbonyl, di-(C₁-C₈-alkyl)aminocarbonyl, C₁-C₈-alkoxycarbonyloxy, C₁-C₈-haloalkoxycarbonyloxy, C₂-C₈-alkenyloxycarbonyloxy, C₂-C₈-haloalkenyloxycarbonyloxy, C₂-C₈-alkynyloxycarbonyloxy, C₃-C₈-haloalkynyloxycarbonyloxy, C₃-C₈-cycloalkoxycarbonyloxy, cycloalkenyloxycarbonyloxy, aminocarbonyloxy, C₁-C₈-alkylaminocarbonyloxy and di-(C₁-C₈-alkyl)aminocarbonyloxy;
R⁷ and R⁸ independently of one another are hydrogen, C₁-C₁₀-alkyl, C₂-C₁₀-alkenyl, C₂-C₁₀-alkynyl, C₃-C₁₀-cycloalkyl, C₃-C₁₀-cycloalkenyl, phenyl, naphthyl or a saturated or unsaturated aromatic or non-aromatic 5-, 6-, 7-, 8-, 9- or 10-membered heterocycle, where the heterocycle contains 1, 2, 3 or 4 heteroatoms selected from the group consisting of O, S and N as ring members and may furthermore contain 1 or 2 CO groups as ring members, where the aliphatic, alicyclic, aromatic and/or heterocyclic groups in R⁷ and/or R⁸ may be partially or fully halogenated and/or may carry 1, 2, 3 or 4 identical or different substituents L⁴;
L¹ is a group of the formula —Y¹—Y²-T in which
   Y¹ is CR^{h}Rⁱ, C(O)O, C(O)NR^{h}, O, NR^{h} or S(O)ᵣ;
   Y² is C₁-C₈-alkylene, C₂-C₈-alkenylene or C₂-C₈-alkynylene, where Y² may be interrupted by one, two, three or four heteroatoms from the group consisting of NR^{h}, O and S(O)ᵣ;
   r is 0, 1 or 2;
   T is halogen, OR^{h}, NR^{h}Rⁱ, C(O)OR^{h}, C(O)NR^{h}Rⁱ, C(NOR^{h})Rⁱ or T¹-C(=T²)-T³ in which
      T¹ is O or NR^{h};
      T² is O, S or NR^{h};
      T³ is R^{h}, OR^{h}, SR^{h} or NR^{h}Rⁱ;
      each R^{h} and Rⁱ is independently H, C₁-C₈-alkyl, C₂-C₈-alkenyl, C₂-C₈-alkynyl, C₃-C₆-cycloalkyl, C₃-C₆-cycloalkenyl, phenyl or a 5- or 6-membered heteroaromatic radical, where the heteroaromatic radical contains 1, 2, 3 or 4 heteroatoms selected from the group consisting of O, S and N as ring members, where phenyl and the heteroaromatic radical may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy,
         or R^{h} and Rⁱ together with the nitrogen atom to which they are attached in the radical NR^{h}Rⁱ form a 5- or 6-membered saturated, partially unsaturated or aromatic heterocycle which may contain 1, 2 or 3 further heteroatoms selected from the group consisting of N, O and S and/or 1 or 2 carbonyl groups as ring members and/or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy;
each L² is independently halogen, hydroxyl, mercapto (SH), cyano, cyanato (OCN), nitro, carboxyl (COOH), C₁-C₁₀-alkyl, C₁-C₁₀-haloalkyl, C₂-C₁₀-hydroxyalkyl, C₁-C₁₀-alkoxy, C₁-C₁₀-haloalkoxy, C₁-C₁₀-alkylthio, C₂-C₁₀-alkenyl, C₂-C₁₀-haloalkenyl, C₂-C₁₀-alkenyloxy, C₂-C₁₀-alkynyl, C₃-C₁₀-haloalkynyl, C₂-C₁₀-alkynyloxy, C₃-C₁₀-cycloalkyl, C₃-C₁₀-cycloalkoxy, C₃-C₁₀-cycloalkyl-C₁-C₄-alkyl, C₃-C₁₀-cycloalkenyl, C₁-C₁₀-alkoxycarbonyl, C₁-C₁₀-haloalkoxycarbonyl, C₂-C₁₀-alkenyloxycarbonyl, C₂-C₁₀-alkynyloxycarbonyl, C₁-C₁₀-alkylcarbonyloxy, C₁-C₁₀-alkenylcarbonyloxy, C₁-C₁₀-alkynylcarbonyloxy, aminocarbonyl, C₁-C₁₀-alkylaminocarbonyl, di-(C₁-C₁₀-alkyl)aminocarbonyl, C₁-C₁₀-alkoximinoalkyl, C₂-C₁₀-alkenyloximinoalkyl, C₂-C₁₀-alkynyloximinoalkyl, formyl, C₁-C₁₀-alkylcarbonyl, C₂-C₁₀-alkenylcarbonyl, C₂-C₁₀-alkynylcarbonyl, C₃-C₆-cycloalkylcarbonyl, NR^{j}R^{k}, NR^{j}—(C═O)—R^{k}, S(═O)ₙA¹, C(═S)A², a group —C(═N—OR^{l})(NR^{m}Rⁿ) or a group —C(═N—NR^{o}R^{p})(NR^{q}R^{r});
   in which
   R^{j}, R^{k}, R^{l}, R^{m}, Rⁿ, R^{o}, R^{p}, R^{q}, R^{r} are each independently H, C₁-C₈-alkyl, C₁-C₈-haloalkyl, C₂-C₈-hydroxyalkyl, C₂-C₈-alkenyl, C₂-C₈-haloalkenyl, C₂-C₈-alkynyl, C₃-C₈-cycloalkyl or C₃-C₈-cycloalkenyl; or
      R^{m} and Rⁿ, R^{o} and R^{p} and/or R^{q} and R^{r} together with the nitrogen atom to which they are attached form a four-, five- or six-membered saturated or partially unsaturated ring which may carry one, two, three or four substituents independently of one another selected from L⁵;
   A¹ is hydrogen, hydroxyl, C₁-C₈-alkyl, amino, C₁-C₈-alkylamino or di-(C₁-C₈-alkyl)amino;
   A² is C₂-C₈-alkenyl, C₁-C₈-alkoxy, C₁-C₆-haloalkoxy, C₂-C₁₀-alkenyloxy, C₂-C₁₀-alkynyloxy or one of the groups mentioned under A¹; and
   n is 0, 1 or 2;
each L³ is independently defined like L² or is phenyl, naphthyl or a saturated or unsaturated aromatic or non-aromatic 5-, 6-, 7-, 8-, 9- or 10-membered heterocycle, where the heterocycle contains 1, 2, 3 or 4 heteroatoms selected from the group consisting of O, S and N as ring members and may furthermore contain one or two CO groups as ring members, where the aliphatic, alicyclic, aromatic and heterocyclic groups in L³ for their part may be partially or fully halogenated and/or may carry 1, 2 or 3 substituents L⁴; each L⁴ is independently cyano, nitro, hydroxyl, mercapto, amino, carboxyl, aminocarbonyl, aminothiocarbonyl, C₁-C₆-alkyl, C₁-C₆-haloalkyl, C₂-C₈-alkenyl, C₄-C₈-alkadienyl, C₂-C₈-alkenyloxy, C₂-C₈-alkynyloxy, C₁-C₆-alkoxy, C₁-C₆-haloalkoxy, C₁-C₆-alkylthio, C₁-C₆-alkylamino, di-(C₁-C₆-alkyl)amino, formyl, C₁-C₆-alkylcarbonyl, C₁-C₆-alkylsulfonyl, C₁-C₆-alkylsulfinyl, C₁-C₆-alkoxycarbonyl, C₁-C₆-alkylcarbonyloxy, C₁-C₆-alkylaminocarbonyl, di-(C₁-C₆-alkyl)aminocarbonyl, C₁-C₆-alkylaminothiocarbonyl, di-(C₁-C₆-alkyl)aminothiocarbonyl, C₃-C₈-cycloalkyl, bicycloalkyl, C₃-C₈-cycloalkoxy, heterocyclyl, heterocyclyloxy, aryl, aryloxy, arylthio, aryl-C₁-C₆-alkoxy or aryl-C₁-C₆-alkyl, where the heterocyclyl radicals may be saturated or unsaturated, aromatic or non-aromatic and have 5, 6, 7, 8, 9 or 10 ring members and 1, 2, 3 or 4 heteroatoms selected from the group consisting of O, S and N and optionally one or two carbonyl groups as ring members and where the cyclic systems may be partially or fully halogenated and/or substituted by C₁-C₆-alkyl or C₁-C₆-haloalkyl groups; and

each L⁵ is in each case independently selected from the group consisting of hydroxyl, cyano, nitro, C₁-C₈-alkyl, C₁-C₈-haloalkyl, C₂-C₈-hydroxyalkyl, C₁-C₈-alkoxy, C₁-C₈-haloalkoxy, C₁-C₈-alkylthio, C₂-C₈-alkenyl, C₂-C₈-haloalkenyl, C₂-C₈-alkenyloxy, C₂-C₈-alkynyl, C₂-C₈-alkynyloxy, C₃-C₈-cycloalkyl, amino, C₁-C₈-alkylamino and di-(C₁-C₈-alkyl)amino;

and/or the agriculturally acceptable salts thereof
for controlling harmful fungi.

Some of the compounds I described above are novel. Accordingly, the present invention also provides novel pyrimidine compounds of the formula I described in more detail below, and fungicidal or pharmaceutical compositions comprising these compounds and/or their agriculturally or pharmaceutically acceptable salts and suitable carriers. Suitable agriculturally and/or pharmaceutically acceptable carriers are described below. Moreover, the invention provides the use of the novel pyrimidine compounds for preparing a medicament for the treatment of cancer.

The invention provides novel pyrimidine compounds of the formula I in which the variables have the general meanings given above or the preferred meanings given below, except for compounds in which
R¹ is NR⁵R⁶, in which R⁵ is H and R⁶ is C₃-C₆-haloalkyl, or is C₃-C₁₀-cycloalkyl and simultaneously
R² is phenyl which carries a substituent L¹ of the formula —Y¹—Y²-T in which Y¹ is O, NR^{h} or S, Y² is C₁-C₄-alkylene and T is OR^{h} or NR^{h}Rⁱ and optionally one or two substituents L² selected from the group consisting of halogen,
R³ is halogen and
R⁴ is NR^{a}R^{b}, NR^{a}—CN, phenyl, naphthyl or 5- to 10-membered hetaryl.

The invention furthermore provides pyrimidine compounds of the formula I in which R¹, R³ and R⁴ have the general meanings given above or the preferred meanings given below and R² is a 5- or 6-membered heteroaromatic radical, where the heteroaromatic radical contains 1, 2, 3 or 4 heteroatoms selected from the group consisting of O, S and N as ring members, carries a substituent L¹ and optionally 1, 2, 3 or 4 identical or different substituents L², where L¹ and L² have the general meanings given above or the preferred meanings given below.

The invention further provides pyrimidine compounds of the formula I in which R¹, R², R³ and R⁴ have the general meanings given above or the preferred meanings given below, but where L¹ is a radical L¹¹ or L¹³. The radicals L¹¹ and L¹³ are defined below.

Moreover, the invention provides pyrimidine compounds of the formula I in which R¹, R² and R³ have the general meanings given above or the preferred meanings given below and R⁴ is a radical of the formula —ON(═CR^{a}R^{b}), —NR^{c}N═CR^{a}R^{b}, —N═OR^{a}; —NR^{c}C(═W)—NR^{a}R^{b}, —NR^{a}C(═W)R^{c}, —NNR^{a}R^{b}C(═W)—X¹—R^{c}, —OC(═W)R^{c}, —O(C═W)NR^{a}R^{b}, —C(═W)R^{c}, —C(═W)NR^{a}R^{b}, —C(═W)NR^{a}OR^{b}, —CR^{a}R^{b}—C(═W)R^{c}, —C(═W)—NR^{a}—X²—R^{b}, —C(═NX²R^{a})—OR^{b} or —C(═NX²R^{a})—SR^{b} in which R^{a}, R^{b}, R^{c}, W, X¹ and X² have the general meanings given above or the preferred meanings given below.

The invention furthermore provides pyrimidine compounds of the formula I in which R¹, R² and R³ have the general meanings given above or the preferred meanings given below and R⁴ is 3-, 4-, 5-, 6-, 7-, 8-, 9- or 10-membered saturated or partially unsaturated heterocyclyl having 1, 2, 3 or 4 heteroatoms selected from the group consisting of O, N and S and optionally 1 or 2 carbonyl groups as ring members, where the heterocyclyl radical may be partially or fully halogenated and/or may have 1, 2 or 3 substituents R^{x} and R^{x} has the general meanings given above or the preferred meanings given below.

The invention further provides pyrimidine compounds of the formula I in which R², R³ and R⁴ have the general meanings given above or the preferred meanings given below and R¹ is C₁-C₁₀-alkyl, C₂-C₁₀-alkenyl, C₂-C₁₀-alkynyl, phenyl, naphthyl or a saturated or unsaturated aromatic or non-aromatic 5-, 6-, 7-, 8-, 9- or 10-membered heterocycle, where the heterocycle contains 1, 2, 3 or 4 heteroatoms selected from the group consisting of O, S and N as ring members and may furthermore contain 1 or 2 CO groups as ring members, where R¹ may be partially or fully halogenated and/or may carry 1, 2, 3 or 4 identical or different substituents L³, where L³ has the general meanings given above or the preferred meanings given below.

Moreover, the invention provides pyrimidine compounds of the formula I in which R², R³ and R⁴ have the general meanings given above or the preferred meanings given below and R¹ is a radical of the formula NR⁵R⁶, where R⁵ and R⁶ have the general meanings given above or the preferred meanings given below, with the proviso that neither R⁵ nor R⁶ is H.

The invention furthermore provides pyrimidine compounds of the formula I in which R², R³ and R⁴ have the general meanings given above or the preferred meanings given below and R¹ is a radical of the formula OR⁷ or SR⁸, where R⁷ and R⁸ have the general meanings given above or the preferred meanings given below.

Depending on the substitution pattern, the compounds of the formula I may have one or more centers of chirality, in which case they are present as mixtures of enantiomers or diastereomers. The invention provides both the pure enantiomers or diastereomers and their mixtures and the use according to the invention of the pure enantiomers or diastereomers of the compound I or its mixtures. Suitable compounds of the formula I also include all possible stereoisomers (cis/trans isomers) and mixtures thereof.

Suitable agriculturally useful salts are especially the salts of those cations or the acid addition salts of those acids whose cations and anions, respectively, have no adverse effect on the fungicidal action of the compounds I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of C₁-C₄-alkanoic acids, preferably formate, acetate, propionate and butyrate. They can be formed by reacting I with an acid of the corresponding anion, preferably hydrochloric acid, hydrobromic acid, sulfuric acid, phosphoric acid or nitric acid.

Suitable pharmaceutically acceptable salts are especially physiologically tolerated salts of the compound I, in particular the acid addition salts with physiologically acceptable acids. Examples of suitable organic and inorganic acids are hydrochloric acid, hydrobromic acid, phosphoric acid, sulfuric acid, C₁-C₄-alkylsulfonic acids, such as methanesulfonic acid, aromatic sulfonic acids, such as benzenesulfonic acid and toluenesulfonic acid, oxalic acid, maleic acid, fumaric acid, lactic acid, tartaric acid, adipic acid and benzoic acid. Further suitable acids are described, for example, in Fortschritte der Arzneimittelforschung, Volume 10, pages 224 ff., Birkhäuser Verlag, Basle and Stuttgart, 1966, the entire contents of which is expressly incorporated herein by way of reference.

In the definitions of the variables given in the formulae above, collective terms are used which are generally representative for the substituents in question. The term Cₙ-Cₘ indicates the number of carbon atoms possible in each case in the substituent or substitutent moiety in question:

halogen: fluorine, chlorine, bromine and iodine;
alkyl and the alkyl moieities in alkoxy, alkylcarbonyl, alkylthiocarbonyl, alkylcarbonyloxy, alkylthiocarbonyloxy, alkylamino, dialkylamino, alkylaminocarbonyl, dialkylaminocarbonyl, alkylaminothiocarbonyl, dialkylaminothiocarbonyl, alkylaminocarbonyloxy, dialkylaminocarbonyloxy, alkylaminothiocarbonyloxy, dialkylaminothiocarbonyloxy, alkylthio, alkylsulfinyl, alkylsulfonyl and the like: saturated straight-chain or branched hydrocarbon radicals having 1 to 2, 1 to 4, 1 to 6, 1 to 8 or 1 to 10 carbon atoms. C₁-C₂-Alkyl is methyl or ethyl. C₁-C₄-Alkyl is additionally also, for example, propyl, isopropyl, butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl, 2-ethylhexyl and positional isomers thereof. C₁-C₁₀-Alkyl is additionally also, for example, nonyl, decyl and positional isomers thereof.

Branched C₃-C₈-alkyl: is an alkyl group having 3 to 8 carbon atoms, at least one of which is a secondary or tertiary carbon atom. Examples are isopropyl, tert-butyl, 2-butyl, isobutyl, 2-pentyl, 2-hexyl, 3-methylpentyl, 1,1-dimethylbutyl, 1,2-dimethylbutyl, 1-methyl-1-ethylpropyl and the like.

Haloalkyl: straight-chain or branched alkyl groups having 1 to 2, 1 to 4, 1 to 6, 1 to 8 or 1 to 10 carbon atoms (as mentioned above), where some or all of the hydrogen atoms in these groups may be replaced by halogen atoms as mentioned above: in particular C₁-C₃-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

C₁-C₁₀-hydroxyalkyl: straight-chain or branched alkyl groups having 1 to 2, 1 to 4, 2 to 4, 1 to 6, 2 to 6, 1 to 8, 2 to 8, 1 to 10 or 2 to 10 carbon atoms (as mentioned above), where at least one of the hydrogen atoms is replaced by a hydroxyl group, such as in 2-hydroxyethyl or 3-hydroxypropyl.

Alkenyl and the alkenyl moieties in alkenyloxy, alkenylcarbonyl and the like: monounsaturated straight-chain or branched hydrocarbon radicals having 2 to 4, 2 to 6, 2 to 8, 3 to 8, 2 to 10 or 3 to 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like;

alkadienyl: doubly unsaturated straight-chain or branched hydrocarbon radicals having 4 to 6, 4 to 8 or 4 to 10 carbon atoms and two double bonds in any position, but preferably not cumulated, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like;

haloalkenyl and the haloalkenyl moieties in haloalkenyloxy, haloalkenylcarbonyl and the like: unsaturated straight-chain or branched hydrocarbon radicals having 2 to 4, 2 to 6, 2 to 8 or 2 to 10 carbon atoms and a double bond in any position (as mentioned above), where some or all of the hydrogen atoms in these groups may be replaced by halogen atoms as mentioned above, in particular fluorine, chlorine and bromine, for example chlorovinyl, chloroallyl and the like; alkynyl and the alkynyl moieties in alkynyloxy, alkynylcarbonyl and the like: straight-chain or branched hydrocarbon groups having 2 to 4, 2 to 6, 2 to 8, 3 to 8, 2 to 10 or 3 to 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

haloalkynyl and the haloalkynyl moieties in haloalkynyloxy, haloalkynylcarbonyl and the like: unsaturated straight-chain or branched hydrocarbon radicals having 3 to 4, 3 to 6, 3 to 8 or 3 to 10 carbon atoms and one or two triple bonds in any position (as mentioned above), where some or all of the hydrogen atoms in these groups may be replaced by halogen atoms as mentioned above, in particular fluorine, chlorine and bromine;

cycloalkyl and the cycloalkyl moieties in cycloalkoxy, cycloalkylcarbonyl and the like; monocyclic saturated hydrocarbon groups having 3 to 6, 3 to 8 or 3 to 10 carbon ring members, such as cyclopropyl, cyclobutyl, cyclopentyl, cyclohexyl, cycloheptyl, cyclooctyl, cyclononyl and cyclodecyl;

halocycloalkyl and the halocycloalkyl moieties in halocycloalkoxy, halocycloalkylcarbonyl and the like: monocyclic saturated hydrocarbon groups having 3 to 6, 3 to 8 or 3 to 10 carbon ring members (as mentioned above) in which some or all of the hydrogen atoms may be replaced by halogen atoms as mentioned above, in particular fluorine, chlorine and bromine;

cycloalkyl-C₁-C₄-alkyl: C₁-C₄-alkyl (as defined above) where one hydrogen atom is replaced by a cycloalkyl group, for example cyclopropylmethyl, cyclopentylmethyl, cyclohexylmethyl and the like.

Cycloalkenyl: monocyclic monounsaturated hydrocarbon groups having 3 to 10, 3 to 8, 3 to 6, preferably 5 to 6, carbon ring members, such as cyclopenten-1-yl, cyclopenten-3-yl, cyclohexen-1-yl, cyclohexen-3-yl, cyclohexen-4-yl and the like;

halocycloalkenyl: monocyclic monounsaturated hydrocarbon groups having 3 to 10, 3 to 8, 3 to 6, preferably 5 to 6, carbon ring members (as mentioned above) in which some or all of the hydrogen atoms may be replaced by halogen atoms as mentioned above, in particular fluorine, chlorine and bromine;

bicycloalkyl: a bicyclic hydrocarbon radical having 5 to 10 carbon atoms, such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl, decalin and the like;

alkoxy: an alkyl group attached via oxygen. C₁-C₂-Alkoxy is methoxy or ethoxy. C₁-C₄-Alkoxy is additionally, for example, n-propoxy, 1-methylethoxy (isopropoxy), butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

Haloalkoxy: an alkoxy radical as mentioned above which is partially or fully substituted by fluorine, chlorine, bromine and/or iodine, preferably by fluorine. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

Alkenyloxy: alkenyl as mentioned above which is attached via an oxygen atom, for example C₃-C₆-alkenyloxy, such as 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy;

haloalkenyloxy: an alkenyloxy radical as mentioned above which is partially or fully substituted by fluorine, chlorine, bromine and/or iodine, preferably by fluorine.

Alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;

haloalkynyloxy: an alkynyloxy radical as mentioned above which is partially or fully substituted by fluorine, chlorine, bromine and/or iodine, preferably by fluorine.

Cycloalkoxy: cycloalkyl as mentioned above which is attached via an oxygen atom, for example C₃-C₁₀-cycloalkoxy or C₃-C₈-cycloalkoxy, such as cyclopropoxy, cyclopentoxy, cyclohexoxy, cycloheptoxy, cyclooctoxy and the like;

cycloalkenyloxy: cycloalkenyl as mentioned above which is attached via an oxygen atom, for example C₃-C₁₀-cycloalkenyloxy, C₃-C₈-cycloalkenyloxy or, preferably, C₅-C₆-cycloalkenyloxy, such as cyclopent-1-enoxy, cyclopent-2-enoxy, cyclohex-1-enoxy and cyclohex-2-enoxy;

alkoxyalkyl: alkyl as defined above having 1 to 8, 1 to 6 or 1 to 4, in particular 1 to 3, carbon atoms, in which one hydrogen atom is replaced by an alkoxy group having 1 to 8, 1 to 6 or 1 to 4 carbon atoms, for example methoxymethyl, 2-methoxyethyl, ethoxymethyl, 3-methoxypropyl, 3-ethoxypropyl and the like.

Cyanoalkyl: alkyl as defined above having 1 to 8, 1 to 6 or 1 to 4, in particular 1 to 3, carbon atoms in which one hydrogen atom is replaced by a cyano group;

alkylcarbonyl: group of the formula R—CO— in which R is an alkyl group as defined above, for example C₁-C₁₀-alkyl, C₁-C₈-alkyl, C₁-C₆-alkyl, C₁-C₄-alkyl or C₁-C₂-alkyl. Examples are acetyl, propionyl and the like.

Alkylthiocarbonyl: group of the formula R—CS— in which R is an alkyl group as defined above, for example C₁-C₁₀-alkyl, C₁-C₈-alkyl, C₁-C₆-alkyl, C₁-C₄-alkyl or C₁-C₂-alkyl. Examples are thioacetyl, thiopropionyl and the like.

Haloalkylcarbonyl: group of the formula R—CO— in which R is a haloalkyl group as defined above, for example C₁-C₁₀-haloalkyl, C₁-C₈-haloalkyl, C₁-C₆-haloalkyl, C₁-C₄-haloalkyl or C₁-C₂-haloalkyl. Examples are trifluoroacetyl, trifluoropropionyl and the like.

Haloalkylthiocarbonyl: group of the formula R—CS— in which R is a haloalkyl group as defined above, for example C₁-C₁₀-haloalkyl, C₁-C₈-haloalkyl, C₁-C₆-haloalkyl, C₁-C₄-haloalkyl or C₁-C₂-haloalkyl. Examples are trifluorothioacetyl, trifluorothiopropionyl and the like.

Alkenylcarbonyl: group of the formula R—CO— in which R is an alkenyl group as defined above, for example C₂-C₁₀-alkenyl, C₂-C₈-alkenyl, C₂-C₆-alkenyl or C₂-C₄-alkenyl.

Alkenylthiocarbonyl: group of the formula R—CS— in which R is an alkenyl group as defined above, for example C₂-C₁₀-alkenyl, C₂-C₈-alkenyl, C₂-C₆-alkenyl or C₂-C₄-alkenyl.

Haloalkenylcarbonyl: group of the formula R—CO— in which R is a haloalkenyl group as defined above, for example C₂-C₁₀-haloalkenyl, C₂-C₈-haloalkenyl, C₂-C₆-haloalkenyl or C₂-C₄-haloalkenyl.

Haloalkenylthiocarbonyl: group of the formula R—CS— in which R is a haloalkenyl group as defined above, for example C₂-C₁₀-haloalkenyl, C₂-C₈-haloalkenyl, C₂-C₆-haloalkenyl or C₂-C₄-haloalkenyl.

Alkynylcarbonyl: group of the formula R—CO— in which R is an alkynyl group as defined above, for example C₂-C₁₀-alkynyl, C₂-C₈-alkynyl, C₂-C₆-alkynyl or C₂-C₄-alkynyl.

Alkynylthiocarbonyl: group of the formula R—CS— in which R is an alkynyl group as defined above, for example C₂-C₁₀-alkynyl, C₂-C₈-alkynyl, C₂-C₆-alkynyl or C₂-C₄-alkynyl.

Haloalkynylcarbonyl: group of the formula R—CO— in which R is a haloalkynyl group as defined above, for example C₂-C₁₀-haloalkynyl, C₂-C₈-haloalkynyl, C₂-C₆-haloalkynyl or C₂-C₄-haloalkynyl.

Haloalkynylthiocarbonyl: group of the formula R—CS— in which R is a haloalkynyl group as defined above, for example C₂-C₁₀-haloalkynyl, C₂-C₈-haloalkynyl, C₂-C₆-haloalkynyl or C₂-C₄-haloalkynyl.

Cycloalkylcarbonyl: group of the formula R—CO— in which R is a cycloalkyl group as defined above, for example C₃-C₁₀-cycloalkyl, C₃-C₈-cycloalkyl, C₃-C₆-cycloalkyl or C₅-C₆-cycloalkyl.

Cycloalkylthiocarbonyl: group of the formula R—CS— in which R is a cycloalkyl group as defined above, for example C₃-C₁₀-cycloalkyl, C₃-C₈-cycloalkyl, C₃-C₆-cycloalkyl or C₅-C₆-cycloalkyl.

Cycloalkenylcarbonyl: group of the formula R—CO— in which R is a cycloalkenyl group as defined above, for example C₃-C₁₀-cycloalkenyl, C₃-C₈-cycloalkenyl, C₃-C₆-cycloalkenyl or C₅-C₆-cycloalkenyl.

Cycloalkenylthiocarbonyl: group of the formula R—CS— in which R is a cycloalkenyl group as defined above, for example C₃-C₁₀-cycloalkenyl, C₃-C₈-cycloalkenyl, C₃-C₆-cycloalkenyl or C₅-C₆-cycloalkenyl.

Alkylcarbonyloxy: group of the formula R—CO—O— in which R is an alkyl group as defined above, for example C₁-C₁₀-alkyl, C₁-C₈-alkyl, C₁-C₆-alkyl, C₁-C₄-alkyl or C₁-C₂-alkyl. Examples are acetyloxy, propionyloxy and the like.

Alkylthiocarbonyloxy: group of the formula R—CS—O— in which R is an alkyl group as defined above, for example C₁-C₁₀-alkyl, C₁-C₈-alkyl, C₁-C₆-alkyl, C₁-C₄-alkyl or C₁-C₂-alkyl. Examples are thioacetyloxy, thiopropionyloxy and the like.

Haloalkylcarbonyloxy: group of the formula R—CO—O— in which R is a haloalkyl group as defined above, for example C₁-C₁₀-haloalkyl, C₁-C₈-haloalkyl, C₁-C₆-haloalkyl, C₁-C₄-haloalkyl or C₁-C₂-haloalkyl. Examples are trifluoracetyloxy, trifluoropropionyloxy and the like.

Haloalkylthiocarbonyloxy: group of the formula R—CS—O— in which R is a haloalkyl group as defined above, for example C₁-C₁₀-haloalkyl, C₁-C₈-haloalkyl, C₁-C₆-haloalkyl, C₁-C₄-haloalkyl or C₁-C₂-haloalkyl. Examples are trifluorothioacetyloxy, trifluorothiopropionyloxy and the like.

Alkenylcarbonyloxy: group of the formula R—CO—O— in which R is an alkenyl group as defined above, for example C₂-C₁₀-alkenyl, C₂-C₈-alkenyl, C₂-C₆-alkenyl or C₂-C₄-alkenyl.

Alkenylthiocarbonyloxy: group of the formula R—CS—O— in which R is an alkenyl group as defined above, for example C₂-C₁₀-alkenyl, C₂-C₈-alkenyl, C₂-C₆-alkenyl or C₂-C₄-alkenyl.

Haloalkenylcarbonyloxy: group of the formula R—CO—O— in which R is a haloalkenyl group as defined above, for example C₂-C₁₀-haloalkenyl, C₂-C₈-haloalkenyl, C₂-C₆-haloalkenyl or C₂-C₄-haloalkenyl.

Haloalkenylthiocarbonyloxy: group of the formula R—CS—O— in which R is a haloalkenyl group as defined above, for example C₂-C₁₀-haloalkenyl, C₂-C₈-haloalkenyl, C₂-C₆-haloalkenyl or C₂-C₄-haloalkenyl.

Alkynylcarbonyloxy: group of the formula R—CO—O— in which R is an alkynyl group as defined above, for example C₂-C₁₀-alkynyl, C₂-C₈-alkynyl, C₂-C₆-alkynyl or C₂-C₄-alkynyl.

Alkynylthiocarbonyloxy: group of the formula R—CS—O— in which R is an alkynyl group as defined above, for example C₂-C₁₀-alkynyl, C₂-C₈-alkynyl, C₂-C₆-alkynyl or C₂-C₄-alkynyl.

Haloalkynylcarbonyloxy: group of the formula R—CO—O— in which R is a haloalkynyl group as defined above, for example C₂-C₁₀-haloalkynyl, C₂-C₈-haloalkynyl, C₂-C₆-haloalkynyl or C₂-C₄-haloalkynyl.

Haloalkynylthiocarbonyloxy: group of the formula R—CS—O— in which R is a haloalkynyl group as defined above, for example C₂-C₁₀-haloalkynyl, C₂-C₈-haloalkynyl, C₂-C₆-haloalkynyl or C₂-C₄-haloalkynyl.

Cycloalkylcarbonyloxy: group of the formula R—CO—O— in which R is a cycloalkyl group as defined above, for example C₃-C₁₀-cycloalkyl, C₃-C₈-cycloalkyl, C₃-C₆-cycloalkyl or C₅-C₆-cycloalkyl.

Cycloalkylthiocarbonyloxy: group of the formula R—CS—O— in which R is a cycloalkyl group as defined above, for example C₃-C₁₀-cycloalkyl, C₃-C₈-cycloalkyl, C₃-C₆-cycloalkyl or C₅-C₆-cycloalkyl.

Cycloalkenylcarbonyloxy: group of the formula R—CO—O— in which R is a cycloalkenyl group as defined above, for example C₃-C₁₀-cycloalkenyl, C₃-C₈-cycloalkenyl, C₃-C₆-cycloalkenyl or C₅-C₆-cycloalkenyl.

Cycloalkenylthiocarbonyloxy: group of the formula R—CS—O— in which R is a cycloalkenyl group as defined above, for example C₃-C₁₀-cycloalkenyl, C₃-C₈-cycloalkenyl, C₃-C₆-cycloalkenyl or C₅-C₆-cycloalkenyl.

Alkoxycarbonyl: group of the formula R—CO— in which R is an alkoxy group as defined above, for example C₁-C₁₀-alkoxy, C₁-C₈-alkoxy, C₁-C₆-alkoxy, C₁-C₄-alkoxy or C₁-C₂-alkoxy. Examples are methoxycarbonyl, ethoxycarbonyl and the like.

Alkoxythiocarbonyl: group of the formula R—CS— in which R is an alkoxy group as defined above, for example C₁-C₁₀-alkoxy, C₁-C₈-alkoxy, C₁-C₆-alkoxy, C₁-C₄-alkoxy or C₁-C₂-alkoxy. Examples are methoxythiocarbonyl, ethoxythiocarbonyl and the like.

Haloalkoxycarbonyl: group of the formula R—CO— in which R is a haloalkoxy group as defined above, for example C₁-C₁₀-haloalkoxy, C₁-C₈-haloalkoxy, C₁-C₆-haloalkoxy, C₁-C₄-haloalkoxy or C₁-C₂-haloalkoxy. Examples are trifluoromethoxycarbonyl, trifluoroethoxycarbonyl and the like.

Haloalkoxythiocarbonyl: group of the formula R—CS— in which R is a haloalkoxy group as defined above, for example C₁-C₁₀-haloalkoxy, C₁-C₈-haloalkoxy, C₁-C₆-haloalkoxy, C₁-C₄-haloalkoxy or C₁-C₂-haloalkoxy. Examples are trifluoromethoxythiocarbonyl, trifluoroethoxythiocarbonyl and the like.

Alkenyloxycarbonyl: group of the formula R—CO— in which R is an alkenyloxy group as defined above, for example C₂-C₁₀-alkenyloxy, C₂-C₈-alkenyloxy, C₂-C₆-alkenyloxy or C₂-C₄-alkenyloxy.

Alkenyloxythiocarbonyl: group of the formula R—CS— in which R is an alkenyloxy group as defined above, for example C₂-C₁₀-alkenyloxy, C₂-C₈-alkenyloxy, C₂-C₆-alkenyloxy or C₂-C₄-alkenyloxy.

Haloalkenyloxycarbonyl: group of the formula R—CO— in which R is a haloalkenyloxy group as defined above, for example C₂-C₁₀-haloalkenyloxy, C₂-C₈-haloalkenyloxy, C₂-C₆-haloalkenyloxy or C₂-C₄-haloalkenyloxy.

Haloalkenyloxythiocarbonyl: group of the formula R—CS— in which R is a haloalkenyloxy group as defined above, for example C₂-C₁₀-haloalkenyloxy, C₂-C₈-haloalkenyloxy, C₂-C₆-haloalkenyloxy or C₂-C₄-haloalkenyloxy.

Alkynyloxycarbonyl: group of the formula R—CO— in which R is an alkynyloxy group as defined above, for example C₂-C₁₀-alkynyloxy, C₂-C₈-alkynyloxy, C₂-C₆-alkynyloxy or C₂-C₄-alkynyloxy.

Alkynyloxythiocarbonyl: group of the formula R—CS— in which R is an alkynyloxy group as defined above, for example C₂-C₁₀-alkynyloxy, C₂-C₈-alkynyloxy, C₂-C₆-alkynyloxy or C₂-C₄-alkynyloxy.

Haloalkynyloxycarbonyl: group of the formula R—CO— in which R is a haloalkynyloxy group as defined above, for example C₂-C₁₀-haloalkynyloxy, C₂-C₈-haloalkynyloxy, C₂-C₆-haloalkynyl or C₂-C₄-haloalkynyloxy.

Haloalkynyloxythiocarbonyl: group of the formula R—CS— in which R is a haloalkynyloxy group as defined above, for example C₂-C₁₀-haloalkynyloxy, C₂-C₈-haloalkynyloxy, C₂-C₆-haloalkynyl or C₂-C₄-haloalkynyloxy.

Cycloalkyloxycarbonyl: group of the formula R—CO— in which R is a cycloalkyloxy group as defined above, for example C₃-C₁₀-cycloalkyloxy, C₃-C₈-cycloalkyloxy, C₃-C₆-cycloalkyloxy or C₅-C₆-cycloalkyloxy.

Cycloalkyloxythiocarbonyl: group of the formula R—CS— in which R is a cycloalkyloxy group as defined above, for example C₃-C₁₀-cycloalkyloxy, C₃-C₈-cycloalkyloxy, C₃-C₆-cycloalkyloxy or C₅-C₆-cycloalkyloxy.

Cycloalkenyloxycarbonyl: group of the formula R—CO— in which R is a cycloalkenyloxy group as defined above, for example C₃-C₁₀-cycloalkenyloxy, C₃-C₈-cycloalkenyloxy, C₃-C₆-cycloalkenyloxy or C₅-C₆-cycloalkenyloxy.

Cycloalkenyloxythiocarbonyl: group of the formula R—CS— in which R is a cycloalkenyloxy group as defined above, for example C₃-C₁₀-cycloalkenyloxy, C₃-C₈-cycloalkenyloxy, C₃-C₆-cycloalkenyloxy or C₅-C₆-cycloalkenyloxy.

Alkoxycarbonyloxy: group of the formula R—CO—O— in which R is an alkoxy group as defined above, for example C₁-C₁₀-alkoxy, C₁-C₈-alkoxy, C₁-C₆-alkoxy, C₁-C₄-alkoxy or C₁-C₂-alkoxy. Examples are methoxycarbonyl, ethoxycarbonyl and the like.

Alkoxythiocarbonyloxy: group of the formula R—CS—O— in which R is an alkoxy group as defined above, for example C₁-C₁₀-alkoxy, C₁-C₈-alkoxy, C₁-C₆-alkoxy, C₁-C₄-alkoxy or C₁-C₂-alkoxy. Examples are methoxycarbonyl, ethoxycarbonyl and the like.

Haloalkoxycarbonyloxy: group of the formula R—CO—O— in which R is a haloalkoxy group as defined above, for example C₁-C₁₀-haloalkoxy, C₁-C₈-haloalkoxy, C₁-C₆-haloalkoxy, C₁-C₄-haloalkoxy or C₁-C₂-haloalkoxy. Examples are trifluoromethoxycarbonyl, trifluoroethoxycarbonyl and the like.

Haloalkoxythiocarbonyloxy: group of the formula R—CS—O— in which R is a haloalkoxy group as defined above, for example C₁-C₁₀-haloalkoxy, C₁-C₈-haloalkoxy, C₁-C₆-haloalkoxy, C₁-C₄-haloalkoxy or C₁-C₂-haloalkoxy. Examples are trifluoromethoxycarbonyl, trifluoroethoxycarbonyl and the like.

Alkenyloxycarbonyloxy: group of the formula R—CO—O— in which R is an alkenyloxy group as defined above, for example C₂-C₁₀-alkenyloxy, C₂-C₈-alkenyloxy, C₂-C₆-alkenyloxy or C₂-C₄-alkenyloxy.

Alkenyloxythiocarbonyloxy: group of the formula R—CS—O— in which R is an alkenyloxy group as defined above, for example C₂-C₁₀-alkenyloxy, C₂-C₈-alkenyloxy, C₂-C₆-alkenyloxy or C₂-C₄-alkenyloxy.

Haloalkenyloxycarbonyloxy: group of the formula R—CO—O— in which R is a haloalkenyloxy group as defined above, for example C₂-C₁₀-haloalkenyloxy, C₂-C₈-haloalkenyloxy, C₂-C₆-haloalkenyloxy or C₂-C₄-haloalkenyloxy.

Haloalkenyloxythiocarbonyloxy: group of the formula R—CS—O— in which R is a haloalkenyloxy group as defined above, for example C₂-C₁₀-haloalkenyloxy, C₂-C₈-haloalkenyloxy, C₂-C₆-haloalkenyloxy or C₂-C₄-haloalkenyloxy.

Alkynyloxycarbonyloxy: group of the formula R—CO—O— in which R is an alkynyloxy group as defined above, for example C₂-C₁₀-alkynyloxy, C₂-C₈-alkynyloxy, C₂-C₆-alkynyloxy or C₂-C₄-alkynyloxy.

Alkynyloxythiocarbonyloxy: group of the formula R—CS—O— in which R is an alkynyloxy group as defined above, for example C₂-C₁₀-alkynyloxy, C₂-C₈-alkynyloxy, C₂-C₆-alkynyloxy or C₂-C₄-alkynyloxy.

Haloalkynyloxycarbonyloxy: group of the formula R—CO—O— in which R is a haloalkynyloxy group as defined above, for example C₂-C₁₀-haloalkynyloxy, C₂-C₈-haloalkynyloxy, C₂-C₆-haloalkynyl or C₂-C₄-haloalkynyloxy.

Haloalkynyloxythiocarbonyloxy: group of the formula R—CS—O— in which R is a haloalkynyloxy group as defined above, for example C₂-C₁₀-haloalkynyloxy, C₂-C₈-haloalkynyloxy, C₂-C₆-haloalkynyl or C₂-C₄-haloalkynyloxy.

Cycloalkyloxycarbonyloxy: group of the formula R—CO—O— in which R is a cycloalkyloxy group as defined above, for example C₃-C₁₀-cycloalkyloxy, C₃-C₈-cycloalkyloxy, C₃-C₆-cycloalkyloxy or C₅-C₆-cycloalkyloxy.

Cycloalkyloxythiocarbonyloxy: group of the formula R—CS—O— in which R is a cycloalkyloxy group as defined above, for example C₃-C₁₀-cycloalkyloxy, C₃-C₈-cycloalkyloxy, C₃-C₆-cycloalkyloxy or C₅-C₆-cycloalkyloxy.

Cycloalkenyloxycarbonyloxy: group of the formula R—CO—O— in which R is a cycloalkenyloxy group as defined above, for example C₃-C₁₀-cycloalkenyloxy, C₃-C₈-cycloalkenyloxy, C₃-C₆-cycloalkenyloxy or C₅-C₆-cycloalkenyloxy.

Cycloalkenyloxythiocarbonyloxy: group of the formula R—CS—O— in which R is a cycloalkenyloxy group as defined above, for example C₃-C₁₀-cycloalkenyloxy, C₃-C₈-cycloalkenyloxy, C₃-C₆-cycloalkenyloxy or C₅-C₆-cycloalkenyloxy.

Alkylamino: group of the formula RHN— in which R is an alkyl group as defined above.

Dialkylamino: group of the formula RRN— in which each R independently is an alkyl group as defined above.

Alkylaminocarbonyl: group of the formula RHN—CO— in which R is an alkyl group as defined above.

Dialkylaminocarbonyl: group of the formula RRN—CO— in which each R independently is an alkyl group as defined above.

Alkylaminothiocarbonyl: group of the formula RHN—CS— in which R is an alkyl group as defined above.

Dialkylaminothiocarbonyl: group of the formula RRN—CS— in which each R independently is an alkyl group as defined above.

Alkylaminocarbonyloxy: group of the formula RHN—CO—O— in which R is an alkyl group as defined above.

Dialkylaminocarbonyloxy: group of the formula RRN—CO—O— in which each R independently is an alkyl group as defined above.

Alkylaminothiocarbonyloxy: group of the formula RHN—CS—O— in which R is an alkyl group as defined above.

Dialkylaminothiocarbonyloxy: group of the formula RRN—CS—O— in which each R independently is an alkyl group as defined above.

Alkylthio: alkyl as defined above which is attached via a sulfur atom.

Haloalkylthio: haloalkyl as defined above which is attached via a sulfur atom. Alkylsulfinyl (sometimes also referred to as alkylsulfoxyl): alkyl as defined above which is attached via an SO group.

Alkylsulfonyl: alkyl as defined above which is attached via an S(O)₂ group.

Aryl: carbocyclic aromatic radical having 6 to 14 carbon atoms, such as phenyl, naphthyl, anthracenyl or phenanthrenyl. C₆-C₁₀-Aryl is phenyl or naphthyl.

Aryloxy: carbocyclic aromatic radical having 6 to 14 carbon atoms which is attached via oxygen, such as phenoxy, naphthyloxy, anthracenyloxy or phenanthrenyloxy. C₆-C₁₀-Aryloxy is phenoxy or naphthoxy.

Arylthio: carbocyclic aromatic radical having 6 to 14 carbon atoms which is attached via sulfur, such as phenylthio, naphthylthio, anthracenylthio or phenanthrenylthio. C₆-C₁₀-Arylthio is phenylthio or naphthylthio.

Arylalkyl: alkyl (as defined above), for example C₁-C₈-alkyl, C₁-C₆-alkyl or C₁-C₄-alkyl, where a hydrogen atom is replaced by an aryl group, such as benzyl, phenethyl and the like.

Arylalkoxy: alkoxy (as defined above), for example C₁-C₈-alkoxy, C₁-C₆-alkoxy or C₁-C₄-alkoxy, where one hydrogen atom is replaced by an aryl group, such as benzyloxy, phenethyloxy and the like.

3-, 4-, 5-, 6-, 7-, 8-, 9- or 10-membered saturated, partially unsaturated or aromatic heterocycle which contains 1, 2, 3 or 4 heteroatoms from the group consisting of oxygen, nitrogen and sulfur and optionally 1 or 2 carbonyl groups as ring members:
three-, four-, five- or six-membered saturated or partially unsaturated heterocycle (hereinbelow also referred to as heterocyclyl) which contains one, two, three or four heteroatoms from the group consisting of oxygen, nitrogen (as N or NR) and sulfur and optionally 1 or 2 carbonyl groups as ring members: for example monocyclic saturated or partially unsaturated heterocycles which, in addition to carbon ring members, contain one to three nitrogen atoms and/or one oxygen or sulfur atom or one or two oxygen and/or sulfur atoms and optionally 1 or 2 carbonyl groups, for example 2-oxiranyl, 2-thiiranyl, 1- or 2-aziridinyl, 1-, 2- or 3-azetidinyl, 2-tetrahydrofuranyl, 3-tetrahydrofuranyl, 3-tetrahydrofuran-2-onyl, 4-tetrahydrofuran-2-onyl, 5-tetrahydrofuran-2-onyl, 2-tetrahydrofuran-3-onyl, 4-tetrahydrofuran-3-onyl, 5-tetrahydrofuran-3-onyl, 2-tetrahydrothienyl, 3-tetrahydrothienyl, 3-tetrahydrothien-2-onyl, 4-tetrahydrothien-2-onyl, 5-tetrahydrothien-2-onyl, 2-tetrahydrothien-3-onyl, 4-tetrahydrothien-3-onyl, 5-tetrahydrothien-3-onyl, 2-pyrrolidinyl, 3-pyrrolidinyl, 1-pyrrolidin-2-onyl, 3-pyrrolidin-2-onyl, 4-pyrrolidin-2-onyl, 5-pyrrolidin-2-onyl, 1-pyrrolidin-3-onyl, 2-pyrrolidin-3-onyl, 4-pyrrolidin-3-onyl, 5-pyrrolidin-3-onyl, 1-pyrrolidin-2,5-dionyl, 3-pyrrolidin-2,5-dionyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals;
seven-membered saturated or partially unsaturated heterocycle which contains one, two, three or four heteroatoms from the group consisting of oxygen, nitrogen and sulfur as ring members: for example mono- and bicyclic heterocycles having 7 ring members which, in addition to carbon ring members, contain one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals;
five- or six-membered aromatic heterocycle (=heteroaromatic radical) which contains one, two, three or four heteroatoms from the group consisting of oxygen, nitrogen and sulfur, for example 5-membered heteroaryl which is attached via carbon and contains one to three nitrogen atoms or one or two nitrogen atoms and one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl, which contains one, two or three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

Alkylene: divalent branched or preferably unbranched chains having 1 to 8 carbon atoms, for example CH₂, CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, CH₂CH₂CH₂CH₂CH₂CH₂CH₂ und CH₂CH₂CH₂CH₂CH₂CH₂CH₂CH₂;

Oxyalkylene: divalent unbranched chains of 2 to 4 CH₂ groups where one valency is attached to the skeleton via an oxygen atom, for example OCH₂CH₂, OCH₂CH₂CH₂ and OCH₂CH₂CH₂CH₂;

oxyalkyleneoxy: divalent unbranched chains of 1 to 3 CH₂ groups where both valencies are attached to the skeleton via an oxygen atom, for example OCH₂O, OCH₂CH₂O and OCH₂CH₂CH₂O;

Alkenylene: aliphatic divalent unbranched chains of 2 to 6 chain members having a C—C double bond in any position, for example CH═CH, CH₂CH═CH, CH₂CH═CHCH₂, CH═CHCH₂CH₂, CH═CHCH₂CH₂CH₂, CH₂CH═CHCH₂CH₂, CH═CHCH₂CH₂CH₂CH₂, CH₂CH═CHCH₂CH₂CH₂ and CH₂CH₂CH═CHCH₂CH₂;

alkynylene: aliphatic divalent unbranched chains of 2 to 6 chain members having a C—C triple bond in any position, for example CH—CH, CH₂C≡C, CH₂C≡CCH₂, C≡CCH₂CH₂, C≡CCH₂CH₂CH₂, CH₂C≡CCH₂CH₂, C≡HCH₂CH₂CH₂CH₂, CH₂C≡CCH₂CH₂CH₂ and CH₂CH₂C≡CCH₂CH₂.

The statements below with respect to suitable and preferred features of the compounds according to the invention and compounds used according to the invention, especially with respect to their substituents R¹, R², R³, R⁴, R⁵, R⁶, R⁷, R⁸, R⁶¹, R⁶², R⁶³, R⁶⁴, R⁶⁵, R⁶⁶, L¹, L², L³, L⁴, L⁵, R^{a1}, R^{b1}, R^{a}, R^{b}, R^{c}, R^{d}, R^{e}, R^{f}, R^{g}, R^{h}, Rⁱ, R^{j}, R^{k}, R^{l}, R^{m}, Rⁿ, Rⁿ, R^{o}, R^{p}, R^{q}, R^{r}, R^{s}, R^{t}, R^{v}, R^{w}, R^{x}, R^{y}, R^{z}, T, T¹, T², T³, W, W¹, X¹, X², Y, Y¹, Y², Z, A, A′, A″, A¹ and A² and the indices a, m, n, q and p and their use, are valid both per se and, in particular, in combination with one another.

With a view to the fungicidal activity, in compounds of the general formula I R¹ is preferably a radical R^{1′} selected from the group consisting of C₁-C₁₀-alkyl, C₂-C₁₀-alkenyl, C₂-C₁₀-alkynyl, C₃-C₁₀-cycloalkyl, C₃-C₁₀-cycloalkenyl, phenyl, naphthyl or a saturated or unsaturated aromatic or non-aromatic 5-, 6-, 7-, 8-, 9- or 10-membered heterocycle, preferably attached via C, where the heterocycle contains 1, 2, 3 or 4 heteroatoms selected from the group consisting of O, S and N as ring members and may furthermore contain 1 or 2 CO groups as ring members, where R^{1′} may be partially or fully halogenated and/or may carry 1, 2, 3 or 4 identical or different substituents L³, which are as defined above. Particularly preferably, R^{1′} is C₁-C₁₀-alkyl, C₃-C₈-alkenyl, C₃-C₈-alkynyl, C₃-C₆-cycloalkyl, C₅-C₆-cycloalkenyl, where the two last-mentioned groups may carry a C₁-C₄-alkylidene group, or is a 5- or 6-membered saturated or aromatic heterocycle which is attached via carbon. R^{1′} may be partially or fully halogenated or carry one, two, three or four identical or different groups L³, which are as defined above.

If R^{1′} carries one, two, three or four, preferably one, two or three, identical or different groups L³, L³ is preferably selected from the group consisting of halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino, C₂-C₆-alkynyloximino, C₃-C₆-cycloalkyl, C₅-C₆-cycloalkenyl, where the aliphatic or alicyclic groups for their part may be partially or fully halogenated or may carry one, two or three groups L⁴.

If L³ carries at least one group L⁴, L⁴ is preferably selected from the group consisting of halogen, cyano, C₁-C₆-alkyl, C₁-C₆-haloalkyl, C₂-C₆-alkenyl, C₂-C₆-alkynyl, C₁-C₆-alkylcarbonyl, C₁-C₆-haloalkylcarbonyl and C₁-C₆-alkoxy.

Particularly preferably, R^{1′} is C₁-C₈-alkyl, especially branched C₃-C₈-alkyl, C₁-C₆-haloalkyl, C₃-C₈-alkenyl, especially branched C₃-C₈-alkenyl, C₃-C₆-cycloalkyl, which may have a C₁-C₄-alkyl group, or C₅-C₆-cycloalkenyl, which may have a C₁-C₄-alkyl group. More preferably, R^{1′} is branched C₃-C₈-alkyl, such as isopropyl, sec-butyl, isobutyl, tert-butyl, 2- and 3-pentyl, 2- and 3-methylbutyl, 1,1-dimethylpropyl, 2,2-dimethylpropyl, 2- and 3-hexyl, 2-, 3- and 4-methylpentyl and the like. The branch point is preferably not at the carbon atom through which the radical R^{1′} is attached to the pyrimidine ring. Examples of such alkyl radicals are isobutyl, 2- and 3-methylbutyl, 2,2-dimethylpropyl, 2-, 3- and 4-methylpentyl and the like.

Alternatively, in compounds of the general formula I, R¹ is preferably a group NR⁵R⁶.

Here, R⁵ is preferably C₁-C₈-alkyl, C₁-C₈-haloalkyl, C₁-C₈-hydroxyalkyl, C₁-C₄-alkoxy-C₁-C₄-alkyl, C₁-C₈-alkyl which carries a substituent selected from the group consisting of COOH, C₁-C₄-alkoxycarbonyl, aminocarbonyl, C₁-C₈-alkylaminocarbonyl, di-(C₁-C₈-alkyl)aminocarbonyl and C₁-C₄-alkylcarbonyloxy, C₃-C₈-cycloalkyl, C₃-C₈-cycloalkyl-C₁-C₄-alkyl or phenyl which optionally carries 1, 2 or 3 substituents selected from the group consisting of halogen, C₁-C₄-alkoxy and C₁-C₄-alkyl.

Particularly preferably, R⁵ is straight-chain or branched C₁-C₈-alkyl or straight-chain or branched C₁-C₈-haloalkyl, straight-chain or branched C₃-C₈-alkyl and straight-chain or branched C₂-C₈-haloalkyl being more preferred. Even more preferably, R⁵ is branched C₃-C₆-alkyl, straight-chain C₂-C₆-haloalkyl or branched C₃-C₆-haloalkyl.

Here, branched C₃-C₆-alkyl is, for example, isopropyl, sec-butyl, isobutyl, tert-butyl, 1-methylpropyl, 2- and 3-pentyl, 2- and 3-methylbutyl, 1,1-dimethylpropyl, 2,2-dimethylpropyl, 1,2-dimethylpropyl, 2- and 3-hexyl, 2-, 3- and 4-methylpentyl, 1,2,2-trimethylpropyl and the like. Particularly preferably, the branched C₃-C₆-alkyl radical has a branching point at the 1-position of the (starting from the nitrogen atom to which the radical R⁵ is attached) longest carbon chain of the alkyl radical, i.e. in the α-position to the nitrogen atom, and optionally a further branching point at a further carbon atom of the alkyl group, in particular at the 2-position of the longest carbon chain of the alkyl radical. Examples of these are isopropyl, sec-butyl, tert-butyl, 1-methylpropyl, 2-pentyl, 2-methylbutyl, 1,1-dimethylpropyl, 1,2-dimethylpropyl, 2-hexyl, 2-methylpentyl, 1,2,2-trimethylpropyl and the like.

The straight-chain or branched C₂-C₈-haloalkyl radical is preferably a fluorinated C₂-C₈-alkyl radical. The fluorinated C₂-C₈-alkyl radical preferably has 1, 2, 3, 4, 5 or 6 fluorine atoms, particularly preferably 1, 2 or 3 and especially 2 or 3 fluorine atoms. Preferably, the fluorine atoms are not attached to the carbon atom of the haloalkyl radical which is attached directly to the nitrogen atom which carries the radical R⁵. Particularly preferably, the fluorine atoms are attached in the 2- and/or 3-position of the (starting from the nitrogen atom to which the radical R⁵ is attached) longest carbon chain of the haloalkyl radical. Preferably, the branched C₃-C₈-haloalkyl radical has a branching point at the 1-position of the (starting from the nitrogen atom to which the radical R⁵ is attached) longest carbon chain of the haloalkyl radical, i.e. at the α-position to the nitrogen atom, and optionally a further branching point at a further carbon atom of the haloalkyl group, for example at the 2- and/or 3-position of the longest carbon chain of the haloalkyl radical.

The straight-chain or branched C₂-C₈-haloalkyl radical is especially a fluorinated C₂-C₃-alkyl radical, for example 2-fluoroethyl, 2,2-difluoroethyl, 2,2,2-trifluoroethyl, 2-fluoro-1-methylethyl, 2,2-difluoro-1-methylethyl, 1-methyl-2,2,2-trifluoroethyl, bis(fluoromethyl)methyl, bis(difluoromethyl)methyl, bis(trifluoromethyl)methyl and the like.

R⁶ is preferably H or has one of the inventive or preferred meanings given for R⁵. Particularly preferably, R⁶ is H or C₁-C₄-alkyl, more preferably H, methyl or ethyl and in particular H or methyl. In a special embodiment of the invention, R⁶ is H.

In an alternative preferred embodiment of the invention, R⁶ is #-CR⁶¹R⁶²—(CR⁶³R⁶⁴)_{q}—(CR⁶⁵R⁶⁶)ₚ—Y-Z in which # is the point of attachment to the nitrogen atom and R⁶¹, R⁶², R⁶³, R⁶⁴, R⁶⁵, R⁶⁶, Y, Z, p and q have the general meanings given above or the preferred meanings given below.

Here R⁶¹ is preferably straight-chain or branched C₁-C₈-alkyl, C₃-C₈-alkenyl or C₃-C₆-cycloalkyl, particularly preferably C₁-C₆-alkyl or C₃-C₆-cycloalkyl, for example methyl, ethyl, propyl, isopropyl, butyl, sec-butyl, isobutyl, tert-butyl, pentyl, sec-pentyl, cyclopropyl or cyclopentyl, preferably isopropyl, isobutyl, tert-butyl, sec-pentyl, cyclopropyl or cyclopentyl and in particular tert-butyl. In an alternative preferred embodiment, R⁶¹ is not hydrogen or methyl. In an alternative preferred embodiment, the group R⁶¹ has a branching point at the α-carbon atom. In an alternative preferred embodiment, the group R⁶¹ is substituted by groups attached via heteroatoms, such as halogen, alkoxy, alkylthio, amino, alkylamino, dialkylamino or formyl, carboxyl, alkoxycarbonyl, alkoxythiocarbonyl or alkenyl, alkynyl groups or C₂-C₅-alkylene, where both valencies are attached to the same carbon atom. In an alternative preferred embodiment, the group R⁶¹ is substituted by C₃-C₆-cycloalkyl or C₃-C₈-cycloalkenyl. In an alternative preferred embodiment, the group R⁶¹ is substituted by C(O)R^{A}, C(O)OR^{A}, C(S)OR^{A}, C(O)NR^{A}R^{B}, C(S)NR^{A}R^{B}, C(NR^{A})R^{B}, C(O)SR^{π} or C(S)SR^{π}.

Here, R^{π} is preferably C₁-C₈-alkyl or C₃-C₆-cycloalkyl, where these groups may be partially or fully halogenated. In an alternative preferred embodiment, the group R⁶¹ is substituted by a five-, six-, seven-, eight-, nine- or ten-membered saturated, partially unsaturated or aromatic heterocycle which contains one, two, three or four heteroatoms from the group consisting of O, N and S.

In a preferred embodiment of the invention, R⁶² is hydrogen, straight-chain or branched C₁-C₈-alkyl or C₃-C₆-cycloalkyl, in particular hydrogen, C₁-C₆-alkyl or C₃-C₆-cycloalkyl, preferably hydrogen, isopropyl or tert-butyl. If R⁶² is an alkyl group, R⁶² preferably has the same meaning as R⁶¹. In an alternative preferred embodiment, R⁶¹ and R⁶² together form a C₃-C₆-alkylene, in particular a C₃-C₄-alkylene, group, where the carbon chains may be substituted by the groups attached via heteroatoms, such as halogen, alkoxy, alkylthio, amino, alkylamino, dialkylamino or alkoxycarbonyl. In an alternative preferred embodiment, R⁶¹ and R⁶² together form a C₃-C₆-alkylene, in particular a C₃-C₄-alkylene, group, where the carbon chains are interrupted by one or two heteroatoms from the group consisting of O, N and S and may be substituted by groups attached via heteroatoms, such as halogen, alkoxy, alkylthio, amino, alkylamino, dialkylamino or alkoxycarbonyl.

In an alternative preferred embodiment, R⁶², R⁶³, R⁶⁴, R⁶⁵ and R⁶⁶ are each hydrogen or C₁-C₄-alkyl, preferably hydrogen, methyl or ethyl, in particular hydrogen. The substitution of the groups R⁶², R⁶³, R⁶⁴, R⁶⁵ and R⁶⁶ corresponds preferably to that of the group R⁶¹.

In an alternative preferred embodiment, R⁶¹ and R⁶³ together form a C₃-C₆-alkylene, C₃-C₆-oxyalkylene or C₂-C₅-oxyalkyleneoxy, in particular a C₃-C₄-alkylene, group.

In an alternative preferred embodiment, R⁶³ and R⁶⁴ and/or R⁶⁵ and R⁶⁶ in each case together form a C₃-C₆-alkylene, C₃-C₆-oxyalkylene or C₂-C₅-oxyalkyleneoxy, in particular a C₃-C₄-alkylene, group.

In a preferred embodiment, the index q has the value zero or 1.

In a preferred embodiment, the index p is zero or 1, in particular zero.

In an alternative preferred embodiment, R⁶³ and R⁶⁴ are preferably hydrogen if the index p has the value zero.

In an alternative preferred embodiment, R⁶⁵ is not hydrogen and R⁶⁶ is hydrogen if the index p is not zero.

In an alternative preferred embodiment, the index p has the value zero or 1 and the index q has the value 1.

In an alternative preferred embodiment, R⁶⁵ and R⁶⁶ are preferably hydrogen. In an alternative preferred embodiment, R⁶⁵ is not hydrogen and R⁶⁶ is hydrogen.

In a preferred embodiment, Y is oxygen.

In one embodiment of the compounds of the formula I, Z is a monovalent group.

In a preferred embodiment, Z is selected from the group consisting of C₁-C₄-alkyl-carbonyl, in particular acetyl, n-propan-1-one, 2-methylpropan-1-one or butan-1-one, hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{π}, C(O)OR^{π}, C(S)OR^{π}, C(O)SR^{π}, C(S)SR^{π}, C(NR^{A})SR^{π}, C(S)R^{π}, C(NR^{π})NR^{A}R^{B}, C(NR^{π})R^{A}, C(NR^{π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkylene-NR^{A}C(NR^{π})NR^{A}R^{B}, C(S)—C₁-C₄-alkylene-NR^{A}C(NR^{π})NR^{A}R^{B}, C(NR^{π})—C₁-C₄-alkylene-NR^{A}C(NR^{π})NR^{A}R^{B}, phenyl, naphthyl, a five-, six-, seven-, eight-, nine- or ten-membered saturated, partially unsaturated or aromatic heterocycle which contains one, two, three or four heteroatoms from the group consisting of O, N and S and which is attached directly or via a carbonyl, thiocarbonyl, C₁-C₄-alkylcarbonyl or C₁-C₄-alkylthiocarbonyl group. The abovementioned groups Z can be substituted by one or more groups R^{b1}. In a further embodiment, the group Z is substituted by one, two, three or four groups R^{b1}, such as halogen, or basic or acidic groups, such as NR^{A}R^{B}, guanidyl, amidyl, hydroxyl, carboxyl or sulfonic acids. Z is especially selected from the group consisting of H, formyl, C₁-C₄-alkylcarbonyl and C₃-C₆-cycloalkylcarbonyl.

Preferably, the groups R^{A} and R^{B} are hydrogen, C₁-C₄-alkyl or C₁-C₄-haloalkyl, in particular hydrogen and methyl.

R^{π} is preferably C₁-C₄-alkyl or C₁-C₄-haloalkyl, in particular methyl.

In a specific embodiment of the invention, in the group #-CR⁶¹R⁶²—(CR⁶³R⁶⁴)_{q}—(CR⁶⁵R⁶⁶)ₚ—Y-Z, R⁶¹ is H or C₁-C₄-alkyl, R⁶² is H, R⁶³ is H or C₁-C₄-alkyl, R⁶⁴ is H, q is 0 or 1, in particular 1, p is 0, Y is O and Z is H, C₁-C₄-alkyl, formyl, C₁-C₄-alkylcarbonyl or C₃-C₆-cycloalkylcarbonyl.

If R⁶ is a group #-CR⁶¹R⁶²—(CR⁶³R⁶⁴)_{q}—(CR⁶⁵R⁶⁶)ₚ—Y-Z, R⁵ is preferably H, C₁-C₈-alkyl or C₁-C₈-haloalkyl, particularly preferably H, C₁-C₄-alkyl or C₁-C₄-haloalkyl and in particular H or C₁-C₄-alkyl.

In a further preferred embodiment of the invention, the group NR⁵R⁶ is ethylglycinol, leucinol, tert-leucinol, valinol, norvalinol, methioninol, phenylalaminol, lysinol, argininol, histidinol, asparaginol, glutaminol, serinol, isoleucinol, cysteinol, hydroxymethylpiperidine, cis-2-hydroxymethyl-4-methylpiperidine, trans-2-hydroxy-methyl-4-methylpiperidine, cyclohexylglycinol, cyclopentylglycinol, butylglycinol, pentylglycinol, cis-2-aminocyclohexanol, trans-2-aminocyclohexanol, cis-2-aminocyclo-pentanol, trans-2-aminocyclopentanol, cis-1-amino-2-hydroxyindane or trans-1-amino-2-hydroxyindane, in each case attached via nitrogen.

In a special embodiment of the invention, neither R⁵ nor R⁶ is H, i.e. the radical R¹ is a tertiary amine.

In an alternative preferred embodiment of the invention, R⁵ and R⁶ together with the nitrogen atom to which they are attached form a saturated or unsaturated 5-, 6-, 7- or 8-membered, preferably 5-, 6- or 7-membered, especially 6- or 7-membered heterocycle, where the heterocycle may additionally contain a heteroatom or a heteroatom-containing group selected from the group consisting of O, N and NR′″ as ring member, where R′″ is H, C₁-C₈-alkyl, C₁-C₈-haloalkyl or C₂-C₈-hydroxyalkyl and in particular H or C₁-C₆-alkyl, and where the heterocycle may carry 1, 2 or 3 substituents selected from the group consisting of halogen, hydroxyl, C₁-C₈-alkyl, C₁-C₈-haloalkyl, C₂-C₈-hydroxyalkyl, C₁-C₈-alkoxy, and C₁-C₈-haloalkoxy. The heterocycle is preferably saturated. Particularly preferably, R⁵ and R⁶ together with the nitrogen atom to which they are attached form a saturated 5-, 6- or 7-membered, and in particular a 6- or 7-membered heterocycle, where the heterocycle may additionally contain a heteroatom or a heteroatom-containing group selected from the group consisting of O and NR′″ as ring member, where R′″ is H, C₁-C₈-alkyl, C₁-C₈-haloalkyl or C₂-C₈-hydroxyalkyl and in particular H or C₁-C₆-alkyl, and where the heterocycle may carry 1 or 2 substituents selected from the group consisting of halogen, hydroxyl, C₁-C₈-alkyl, C₁-C₈-haloalkyl, C₂-C₈-hydroxyalkyl, C₁-C₈-alkoxy and C₁-C₈-haloalkoxy. Preferably, the heterocycle has, in addition to the nitrogen atom which carries the radicals R⁵ and R⁶, no further heteroatoms as ring members. If the heterocycle carries substituents, these are preferably selected from the group consisting of halogen, C₁-C₄-alkyl and C₁-C₄-haloalkyl and in particular from the group consisting of C₁-C₄-alkyl. Especially, the heterocycle is unsubstituted or carries a C₁-C₄-alkyl substituent, for example a methyl substituent.

In an alternative preferred embodiment of the invention, R¹ is a radical OR⁷. In a further alternative preferred embodiment of the invention, R¹ is a radical SR⁸.

Here, R⁷ and R⁸ are preferably not H. Preferably, they are C₁-C₆-alkyl, C₁-C₆-haloalkyl, C₂-C₆-alkenyl, C₂-C₆-alkynyl or C₃-C₆-cycloalkyl. Particularly preferably, they are C₁-C₆-alkyl, C₂-C₆-alkenyl or C₁-C₆-haloalkyl which are in each case branched at the α-position. Alternatively, they are particularly preferably C₁-C₄-haloalkyl. They are in particular ethyl, propyl, isopropyl, 1,2-dimethylpropyl, 1,2,2-trimethylpropyl, 1-methyl-2,2,2-trifluoroethyl or 2,2,2-trifluoroethyl.

In a particularly preferred embodiment of the invention, R¹ is a group R¹′ or a group NR⁵R⁶, where R¹′, R⁵ and R⁶ preferably have the preferred meanings given above. R¹ is in particular a group NR⁵R⁶, where R⁵ and R⁶ preferably have the preferred meanings given above.

In a preferred embodiment of the invention, the radical R² is phenyl, pyridinyl, for example 2-, 3- or 4-pyridinyl, pyrimidinyl, for example 2-, 4- or 5-pyrimidinyl, pyrazinyl, for example 2-pyrazinyl, pyridazinyl, for example 3- or 4-pyridazinyl, triazinyl, furyl, for example 2- or 3-furyl, thienyl, for example 2- or 3-thienyl, pyrrolyl, for example 2- or 3-pyrrolyl, pyrazolyl, for example 1-, 3-, 4- or 5-pyrazolyl, imidazolyl, for example 1-, 2-, 4- or 5-imidazolyl, oxazolyl, for example 2-, 4- or 5-oxazolyl, isoxazolyl, for example 3-, 4- or 5-isoxazolyl, thiazolyl, for example 2-, 4- or 5-thiazolyl, isothiazolyl, for example 3-, 4- or 5-isothiazolyl, triazolyl, for example 1-, 4- or 5-[1,2,3]-1H-triazolyl, 2-, 4- or 5-[1,2,3]-2H-triazolyl, 1-, 3- or 5-[1,2,4]-1H-triazolyl or 3-, 4- or 5-[1,2,4]-4H-triazolyl, oxadiazolyl, for example 4- or 5-[1,2,3]-oxadiazolyl, 3- or 5-[1,2,4]-oxadiazolyl or 2- or 5-[1,3,4]-oxadiazolyl, thiadiazolyl, for example 4- or 5-[1,2,3]-thiadiazolyl, 3- or 5-[1,2,4]-thiadiazolyl or 2- or 5-[1,3,4]-thiadiazolyl, or tetrazolyl, for example 1-, 2- or 5-[1,2,3,4]tetrazolyl which carries a substituent L¹ and 0, 1, 2, 3 or 4, preferably 0, 1 or 2, substituents L², where L¹ and L² are defined as above or, preferably, as described below.

Particularly preferably, the radical R² is phenyl, pyridinyl, for example 2-, 3- or 4-pyridinyl, pyrimidinyl, especially 4- or 5-pyrimidinyl, pyrazinyl, for example 2-pyrazinyl, pyridazinyl, for example 3- or 4-pyridazinyl, furyl, for example 2- or 3-furyl, thienyl, for example 2- or 3-thienyl, pyrazolyl, especially 1- or 5-pyrazolyl, imidazolyl, especially 1-, 2- or 5-imidazolyl, oxazolyl, for example 2-, 4- or 5-oxazolyl, isoxazolyl, for example 3-, 4- or 5-isoxazolyl, thiazolyl, for example 2-, 4- or 5-thiazolyl, isothiazolyl, for example 3-, 4- or 5-isothiazolyl, or triazolyl, especially 1-[1,2,4]-1H-triazolyl which carries a substituent L¹ and 0, 1, 2, 3 or 4, preferably 0, 1 or 2, in particular 1 or 2, substituents L², where L¹ and L² are defined as above, or, preferably, as described below.

In one more preferred embodiment of the invention, R² is phenyl substituted by a radical L¹ and 0, 1, 2, 3 or 4, preferably 1 or 2, in particular 2 radicals L².

Suitable radicals L² are in particular the following groups: halogen, such as fluorine or chlorine; cyano; nitro; alkoxycarbonyl; aminocarbonyl; C₁-C₄-alkyl, such as methyl; C₁-C₄-haloalkyl, such as trifluoromethyl; C₁-C₄-alkoxy, such as methoxy.

Preferred embodiments of the radical R² relate in particular to phenyl groups which, in addition to the group L¹, may have the following substituents (for position numbering see the following illustration):

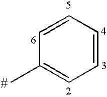

position 2: fluorine, chlorine, methyl; position 3: hydrogen, fluorine, methoxy; position 4: hydrogen, fluorine, chlorine, methyl, methoxy, cyano, nitro, alkoxycarbonyl, aminocarbonyl, haloalkyl, particularly preferably fluorine, chlorine, methyl, methoxy, cyano; position 5: hydrogen, fluorine, chlorine, methyl; particularly preferably hydrogen, fluorine; position 6: hydrogen, fluorine, chlorine, methyl; particularly preferably hydrogen, fluorine.

The group L¹ is preferably located in position 3, 4 or 5, particularly preferably 3 or 4 and in particular 4, relative to the 1-position of the point of attachment to the pyrimidine ring.

In a preferred embodiment of the invention, R² is one of the groups A or B.

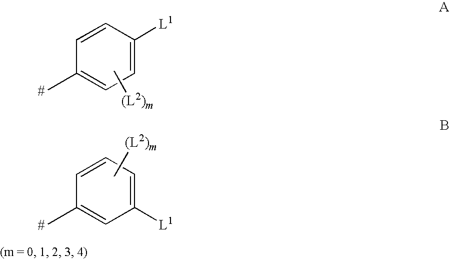

Here, L² is preferably one of the following substituent combinations: 2-Cl; 2-F; 2-CH₃; 2,6-F₂; 2,6-C₁₂; 2-F, 6-CH₃; 2,4,6-F₃; 2,6-F₂-4-OCH₃; 2-C₁₋₄-OCH₃; 2-CH₃-4-F; 2-CF₃; 2-OCH₃,6-F; 2,4-F₂; 2-F-4-Cl; 2-F-6-Cl; 2-C₁₋₄-F; 2-C₁₋₅-F; 2,3-F₂; 2,5-F₂; 2,3,4-F₃; 2-CH₃; 2,4-(CH₃)₂; 2-CH₃-4-Cl; 2-CH₃, 5-F; 2-F, 4-CH₃; 2,6-(CH₃)₂; 2,4,6-(CH₃)₃; 2,6-F₂, 4-CH₃. Particularly preferably, L² is one of the following substituent combinations: 2-F; 2-Cl; 2-CH₃; 2,6-F₂; 2-F, 6-Cl; 2-F, 6-CH₃ and in particular 2,6-F₂.

Group A is particularly preferred.

The compounds of the formula I which carry groups A or B correspond to the formulae I.A and I.B.

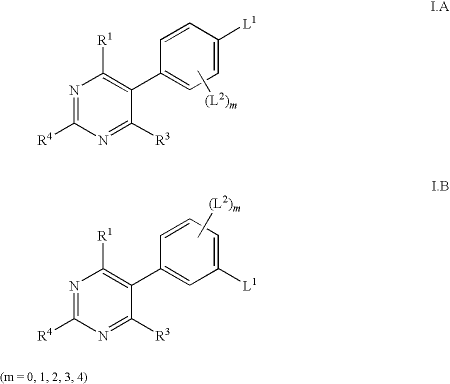

In a further embodiment of the invention, R² is 5-membered heteroaryl which is substituted by a radical L¹ and optionally by 1, 2 or 3 radicals L². Here, the 5-membered heteroaryl ring is preferably selected from the group consisting of thienyl, for example 2- or 3-thienyl, pyrazolyl, for example 1-, 3-, 4- or 5-pyrazolyl, and thiazolyl, for example 2-, 4- or 5-thiazolyl.

In a further embodiment of the invention, R² is 6-membered heteroaryl which contains 1 to 3 nitrogen atoms and is substituted by a radical L¹ and optionally by 1, 2 or 3 radicals L². Here, the 6-membered heteroaryl ring is preferably selected from the group consisting of pyridinyl, for example 2-, 3- or 4-pyridinyl, pyrimidinyl, for example 2-, 4- or 5-pyrimidinyl, pyrazinyl, for example 2-pyrazinyl, and pyridazinyl, for example 3- or 4-pyridazinyl.

In a preferred embodiment of the invention, R² is pyridyl which is attached in the 2-, 3- or 4-position to the pyrimidine ring and may carry 1, 2 or 3 identical or different substituents L² which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred embodiment of such compounds are those of the formulae I.C and I.D.

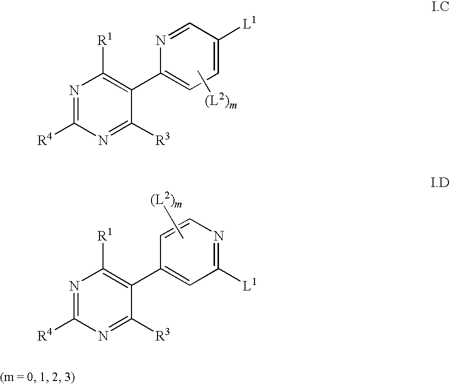

In an alternative preferred embodiment of the invention, R² is pyrimidyl which is attached in the 2- or 4-position to the pyrimidine ring and may carry 1 or 2 identical or different substituents L² which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred embodiment of such compounds are those of the formulae I.E and I.F.

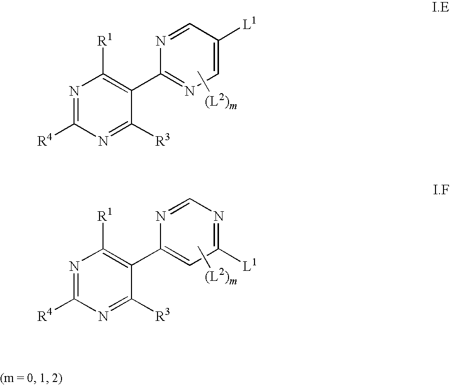

In an alternative preferred embodiment of the invention, R² is thienyl which is attached in the 2- or 3-position to the pyrimidine ring and may carry 1 or 2 identical or different substituents L² which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred embodiment of such compounds are those of the formulae I.G and I.H.

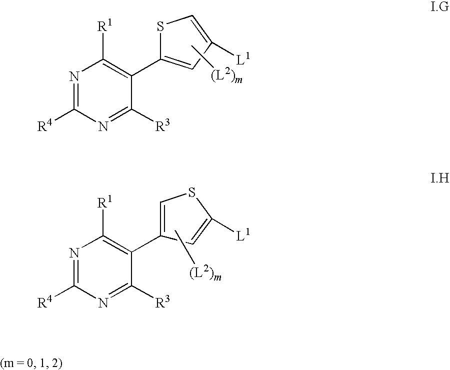

In an alternative preferred embodiment of the invention, R² is thiazolyl which is attached in the 2-, 4- or 5-position to the pyrimidine ring and may carry a substituent L² which is preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred embodiment of such compounds are those of the formulae I.I and I.J.

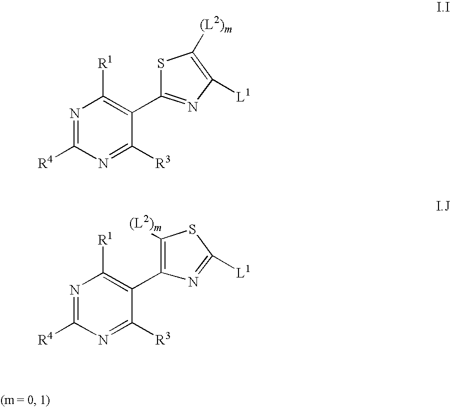

In an alternative preferred embodiment of the invention, R² is imidazolyl which is attached in the 4- or 5-position to the pyrimidine ring and may carry 1 or 2 identical or different substituents L² which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred embodiment of such compounds are those of the formulae I.K and I.L.

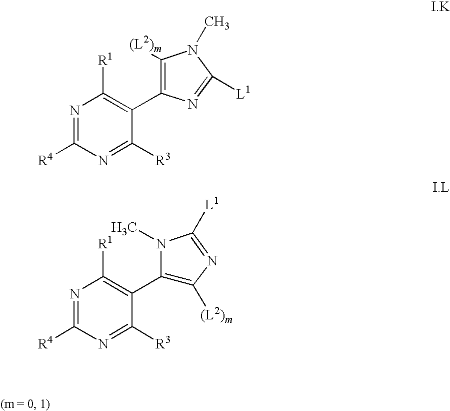

In an alternative preferred embodiment of the invention, R² is pyrazolyl which is attached in the 1-, 3-, 4- or 5-position to the pyrimidine ring and may carry 1 or 2 identical or different substituents L² which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred embodiment of such compounds are those of the formulae I.M, I.N and I.O.

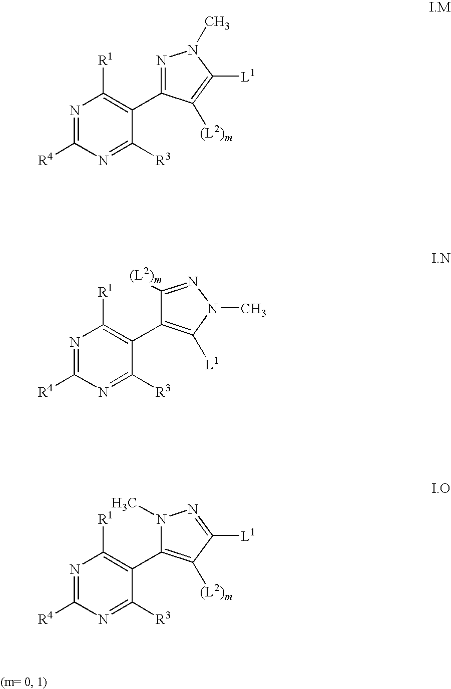

In an alternative preferred embodiment of the invention, R² is oxazolyl which is attached in the 2-, 3- or 4-position to the pyrimidine ring and may carry a substituent L² which is preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred embodiment of such compounds are those of the formulae I.P and I.Q

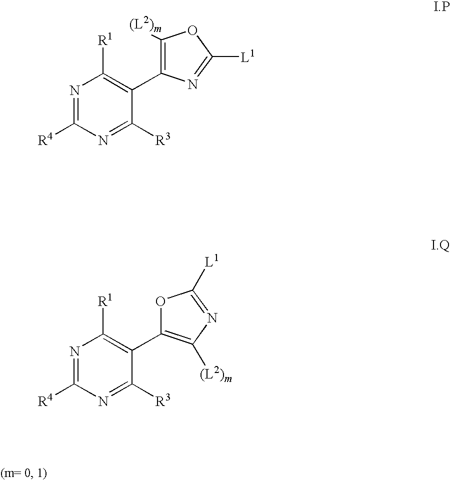

In a preferred embodiment of the compounds I, in particular of the formulae I.A to I.Q, at least one group L² is located in the ortho-position to the point of attachment of the group R² to the pyrimidine skeleton, in particular chlorine, fluorine or methyl.

In a further preferred embodiment, a heteroatom of the heteroaromatic radical R² is located in the ortho-position to the point of attachment.

If structurally possible, the index m is preferably 1 to 4, where the groups L² may be identical or different. If the heteroaromatic groups R² carry, in addition to a group L¹, further substituents L², these are preferably selected from the group consisting of: fluorine, chlorine, methyl, methoxy, cyano, nitro, alkoxycarbonyl, aminocarbonyl and haloalkyl. In a further embodiment, the optional substituents L² are selected from the group consisting of fluorine, chlorine, methyl and methoxy. In a further embodiment, the optional substituents L² are selected from the group consisting of chlorine, methyl and methoxy. A further embodiment relates to heteroaromatic groups R² which, in addition to a group L¹, are substituted by chlorine.

The radical R² is in particular phenyl or pyridinyl, especially 2-pyridinyl, where these carry a substituent L¹ and 0, 1, 2, 3 or 4, preferably 0, 1 or 2, in particular 1 or 2, substituents L², where L¹ and L² are defined as above or as described below.

If R² is phenyl or 2-pyridinyl, these rings preferably carry the substituent L¹ in the 3- or, in particular, 4-position (based on the 1-position of the bond to the pyrimidine ring; i.e. L¹ is particularly preferably attached in the meta- or, in particular, para-position to this point of attachment). The phenyl or the 2-pyridinyl ring optionally has 1 or 2 further substituents L². These are preferably attached in the 2- and/or 6-position of the phenyl ring (based on the 1-position of the bond to the pyrimidine ring), i.e. in the ortho-position to the point of attachment to the pyrimidine ring, and in the case of the 2-pyridine ring preferably attached in the 6-position (based on the 1-position of the bond to the pyrimidine ring).

R² is in particular phenyl. Preferably, the radical L¹ is attached in the 4-position of the phenyl ring, based on the 1-position of the bond of the phenyl ring to the pyrimidine ring. Preferably, the phenyl ring furthermore carries 1 or 2, preferably 2, substituents L² which are preferably attached in the 2- or 2,6-position. Preferred substituents L² are mentioned above; particularly preferably, L² is F.

In a preferred embodiment of the invention, the substituent L¹ of the radical R² is a radical L¹¹ of the formula

-Y^{α1}-[A^{α}-Y^{α2}]ₐ-A^{α}-T^{α}

in which
A^{α} is C₁-C₄-alkylene;
Y^{α1}, Y^{α2} independently of one another are O, S or NR^{hα};
T^{α} is OR^{hα}, SR^{hα} or NR^{hα}R^{iα};
R^{hα} and R^{iα} independently of one another are H or C₁-C₄-alkyl; and
a is 1, 2, 3 or 4.

C₁-C₄-Alkylene in A^{α} is preferably methylene, 1,2-ethylene, 1,2- or 1,3-propylene or 1,4-n-butylene.

A^{α} is preferably methylene, 1,2-ethylene, 1,2-propylene or 1,3-propylene and in particular methylene or 1,2-ethylene.

Y^{α1} and Y^{α2} independently of one another are preferably O or NR^{hα}. If Y^{α1} is O, Y^{α2} is also preferably O. Moreover, in this case T^{α} is preferably OR^{hα}. If Y^{α1} is NR^{hα}R^{iα} and Y^{α2} is simultaneously O, T^{α} is in this case preferably OR^{hα}.

T^{α} is preferably OR^{hα} or NR^{hα}R^{iα}.

R^{hα} and R^{iα} independently of one another are preferably H, methyl or ethyl.

a is preferably 1, 2 or 3.

In another preferred embodiment of the invention, the substituent L¹ of the radical R² is a radical L¹² of the formula

Y^{β}-A^{β}-T^{β}

in which
Y^{β} is CH₂, O, S or NR^{hβ}R^{iβ};
A^{β} is C₁-C₈-alkylene;
T^{β} is halogen, OR^{hβ}, NR^{hβ}R^{iβ}, NR^{hβ}C(═O)-T^{3β} or OC(═O)-T^{3β};
T^{3β} is R^{hβ}, OR^{hβ} or NR^{hβ}OR^{iβ}; and
each R^{hβ} and R^{iβ} is independently H, C₁-C₈-alkyl, C₂-C₈-alkenyl, C₂-C₈-alkynyl, C₃-C₆-cycloalkyl, C₃-C₆-cycloalkenyl, phenyl or a 5- or 6-membered heteroaromatic radical, where the heteroaromatic radical contains 1, 2, 3 or 4 heteroatoms selected from the group consisting of O, S and N as ring members, where phenyl and the heteroaromatic radical may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy,
   or R^{h} and Rⁱ together with the nitrogen atom to which they are attached in the radical NR^{h}Rⁱ form a 5- or 6-membered saturated, partially unsaturated or aromatic heterocycle which may contain 1, 2 or 3 further heteroatoms selected from the group consisting of N, O and S and/or 1 or 2 carbonyl groups as ring members and/or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy.
Y^{β} is preferably CH₂ or O and especially O.
A^{β} is preferably C₁-C₆-alkylene, particularly preferably C₁-C₄-alkylene, in particular 1,2-ethylene or 1,3-propylene and especially 1,3-propylene.
T^{β} is preferably halogen, OR^{hβ}, NR^{hβ}R^{iβ} or NR^{hβ}C(═O)-T^{3β}.

R^{hβ} and R^{iβ} independently of one another are preferably H, C₁-C₆-alkyl, phenyl or a 5- or 6-membered heteroaromatic radical, where the heteroaromatic radical contains 1, 2, 3 or 4 heteroatoms selected from the group consisting of O, S and N as ring members, where phenyl and the heteroaromatic radical may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy; or R^{h} and Rⁱ together with the nitrogen atom to which they are attached in the radical NR^{h}Rⁱ form a 5- or 6-membered saturated, partially unsaturated or aromatic heterocycle which may contain 1, 2 or 3 further heteroatoms selected from the group consisting of N, O and S and/or 1 or 2 carbonyl groups as ring members and/or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy.

Particularly preferably, R^{hβ} and R^{iβ} independently of one another are H, C₁-C₆-alkyl or a 5- or 6-membered heteroaromatic radical, where the heteroaromatic radical contains 1, 2 or 3 heteroatoms selected from a group consisting of O, S and N as ring members, where the heteroaromatic radical may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy; or R^{h} und Rⁱ together with the nitrogen atom to which they are attached in the radical NR^{h}Rⁱ form a 5- or 6-membered saturated, partially unsaturated or aromatic heterocycle which may contain 1, 2 or 3 further heteroatoms selected from the group consisting of N, O and S and/or 1 or 2 carbonyl groups as ring members and/or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy.

More preferably, R^{hβ} and R^{iβ} independently of one another are H, C₁-C₆-alkyl or a 5- or 6-membered heteroaromatic radical, where the heteroaromatic radical contains 1, 2 or 3 nitrogen atoms as ring members, where the heteroaromatic radical may carry 1 or 2 substituents selected from the group consisting of halogen, hydroxyl, C₁-C₄-alkyl, C₁-C₄-haloalkyl, C₁-C₄-alkoxy and C₁-C₄-haloalkoxy; or R^{hβ} and R^{iβ} together with the nitrogen atom to which they are attached in the radical NR^{h}Rⁱ form a 5- or 6-membered saturated or aromatic heterocycle which may contain 1 or 2 further nitrogen atoms and/or 1 or 2 carbonyl groups as ring members and/or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy.

In the radical OR^{hβ}, R^{hβ} is preferably H, C₁-C₄-alkyl or a 5- or 6-membered heteroaromatic radical, where the heteroaromatic radical contains 1, 2 or 3 nitrogen atoms as ring members, where the heteroaromatic radical may carry 1 or 2 substituents selected from the group consisting of halogen, hydroxyl, C₁-C₄-alkyl, C₁-C₄-haloalkyl, C₁-C₄-alkoxy and C₁-C₄-haloalkoxy. In particular, R^{hβ} is methyl, ethyl, pyridyl or pyrimidinyl, where pyridyl and pyrimidyl may carry 1 or 2 substituents selected from the group consisting of halogen, hydroxyl, C₁-C₄-alkyl, C₁-C₄-haloalkyl, C₁-C₄-alkoxy and C₁-C₄-haloalkoxy.

In the radical NR^{hβ}R^{iβ}, R^{hβ} and R^{iβ} are preferably H or C₁-C₄-alkyl, preferably not both radicals being H, or they form, together with the nitrogen atom to which they are attached in the radical NR^{h}Rⁱ, a 5- or 6-membered saturated or aromatic heterocycle which may contain 1 or 2 further nitrogen atoms and/or 1 or 2 carbonyl groups as ring members and/or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy.

In the radical NR^{hβ}C(═O)-T^{3β}, R^{hβ} is preferably H or C₁-C₄-alkyl, in particular methyl. T^{3β} is preferably OR^{hβ}, where R^{hβ} is preferably H or C₁-C₆-alkyl.

In another preferred embodiment of the invention, the substituent L¹ of the radical R² is a radical L¹³ of the formula

Y^{1γ}-A^{γ}-T^{γ}

in which

### Y^{1γ} is —CONR^{hγ} or —COO;

A^{γ} is C₂-C₆-alkylene;

### T^{γ} is OR^{hγ}, NR^{hγ}R^{iγ} or OC(═O)-T^{3γ};

### T^{3γ} is R^{hγ}, OR^{hγ} or NR^{hγ}R^{iγ}; and

each R^{hγ} and R^{1γ} is independently H or C₁-C₄-alkyl.

Particularly preferably, the substituent L¹ of the radical R² is a radical L¹¹ or L¹² and in particular L¹².

L² is preferably halogen, C₁-C₈-alkyl, for example C₁-C₄-alkyl, C₁-C₈-haloalkyl, for example C₁-C₄-haloalkyl, C₁-C₈-alkoxy, for example C₁-C₄-alkoxy, or C₁-C₈-haloalkoxy, for example C₁-C₄-haloalkoxy. Particularly preferably, L² is halogen or C₁-C₄-alkyl and especially halogen, such as chlorine or fluorine, or methyl. Even more especially L² is fluorine.

R³ is preferably halogen, C₁-C₁₀-alkyl, especially C₁-C₈-alkyl, C₁-C₁₀-haloalkyl, especially C₁-C₈-haloalkyl, C₁-C₁₀-alkoxy, especially C₁-C₈-alkoxy, C₁-C₁₀-haloalkoxy, especially C₁-C₈-haloalkoxy, or CN, particularly preferably halogen, C₁-C₄-alkyl, C₁-C₄-haloalkyl, C₁-C₄-alkoxy, C₁-C₄-haloalkoxy or CN, more preferably halogen, C₁-C₄-alkyl, especially C₁-C₂-alkyl, or C₁-C₄-haloalkyl, especially C₁-C₂-haloalkyl. R³ is in particular halogen, especially chlorine, or C₁-C₄-alkyl, especially C₁-C₂-alkyl, in particular methyl, and specifically halogen, especially chlorine.

In a preferred embodiment of the invention, R⁴ is a radical R^{4a} which for its part is a 3-, 4-, 5-, 6-, 7-, 8-, 9- or 10-membered, preferably 5- or 6-membered, saturated, partially unsaturated or aromatic heterocyclic ring having 1, 2, 3 or 4 heteroatoms selected from the group consisting of O, N and S and optionally 1 or 2 carbonyl groups as ring members, where the heterocyclic ring may be partially or fully halogenated and/or may carry 1, 2 or 3 radicals R^{x}, where R^{x} is as defined above.

The 5- or 6-membered heterocycles are preferably selected from the group consisting of pyrrolyl, such as 1-, 2- and 3-pyrrolyl; pyrrolinyl, such as 1-, 2- and 3-pyrrolinyl; pyrrolinonyl, pyrrolidinyl, such as 1-, 2- and 3-pyrrolidinyl; pyrrolidonyl, such as 1-, 3-, 4- and 5-pyrrolidin-2-onyl and also 1-, 2-, 4- and 5-pyrrolidin-3-onyl; pyrrolidindionyl, such as 1-pyrrolidin-2,5-dionyl; pyrazolyl, such as 1-, 3-, 4- and 5-pyrazolyl; pyrazolinyl, such as 1-, 3-, 4- and 5-pyrazolinyl; pyrazolidinyl, such as 1-, 2-, 3- and 4-pyrazolidinyl; pyrazolidinonyl; imidazolyl, such as 1-, 2-, 4- and 5-imidazolyl; imidazolinyl, such as 1-, 2-, 4- and 5-imidazolinyl; imidazolidinyl, such as 1-, 2- and 4-imidazolidinyl; imidazolidinonyl, such as 1- and 4-imidazolidin-2-onyl and also 1-, 2-, 3- and 5-imidazolidin-4-onyl; triazolyl, such as 1- and 2-[1,3,5]-(1H)-triazolyl, 1-[1,2,3]-(1H)-triazolyl, 2-[1,2,3]-(2H)-triazolyl and also 1-, 3- and 5-[1,2,4]-(1H)-triazolyl; tetrazolyl, such as 1- and 5-[1,2,3,4]-(1H)-tetrazolyl; thienyl, such as 2- and 3-thienyl; dihydrothienyl, such as 2,3-dihydrothien-2-, 3-, 4- and 5-yl; tetrahydrothienyl, such as tetrahydrothien-2- or 3-yl; tetrahydrothienonyl, such as tetrahydrothien-2-on-3-, 4- or 5-yl; dithiolanyl, such as 1,3-dithiolan-2- and 4-yl; furanyl, such as 2- and 3-furanyl; dihydrofuranyl, such as 2,3-dihydrofuran-2-, 3-, 4- and 5-yl; tetrahydrofuranyl, such as tetrahydrofuran-2- or 3-yl; tetrahydrofuranonyl, such as tetrahydrofuran-2-on-3-, 4- or 5-yl; tetrahydrofurandionyl, such as tetrahydrofuran-2,5-dion-3-yl; dioxolanyl, such as 1,3-dioxolan-2- and 4-yl; thiazolyl, such as 2-, 4- and 5-thiazolyl; thiazolinyl, such as 2-, 4- and 5-thiazolinyl; thiazolidinyl, such as 2-, 4- and 5-thiazolidinyl; isothiazolyl, such as 3-, 4- and 5-isothiazolyl; isothiazolinyl, such as 3-, 4- and 5-isothiazolinyl; isothiazolidinyl, such as 3-, 4- and 5-isothiazolidinyl; oxazolyl, such as 2-, 4- and 5-oxazolyl; oxazolinyl, such as 2-, 4- and 5-oxazolinyl; oxazolidinyl, such as 2-, 3-, 4- and 5-oxazolidinyl, oxazolidinonyl, such as 3-, 4- and 5-oxazolidin-2-onyl; isoxazolyl, such as 3-, 4- and 5-isoxazolyl; isoxazolinyl, such as 3-, 4- and 5-isoxazolinyl; isoxazolidinyl, such as 3-, 4- and 5-isoxazolidinyl; isoxazolidinonyl, such as 2-, 4- and 5-isoxazolidin-3-onyl; thiadiazolyl, such as 3- and 5-[1,2,4]-thiadiazolyl and also 2- and 5-[1,3,4]-thiadiazolyl; oxadiazolyl, such as 3- and 5-[1,2,4]-oxadiazolyl and also 2- and 5-[1,3,4]-oxadiazolyl; pyridyl, such as 2-, 3- and 4-pyridyl; dihydropyridyl, such as 1,4-dihydropyrid-1-, 2-, 3- and 4-yl; dihydropyridinonyl, such as 1-, 3-, 4-, 5- and 6-(1,2-dihydro)-pyridin-2-onyl; tetrahydropyridyl, such as 1,2,3,6-tetrahydropyrid-1-, 2-, 3-, 4-, 5- and 6-yl and also 1,2,3,4-tetrahydropyrid-1-, 2-, 3-, 4-, 5- and 6-yl; tetrahydropyridinonyl, such as 1-, 3-, 4-, 5- and 6-(1,2,3,4-tetrahydro)pyridin-2-onyl; piperidyl, such as 1-, 2-, 3- and 4-piperidyl; pyrimidinyl, such as 2-, 4- and 5-pyrimidinyl; pyridazinyl, such as 2- and 3-pyridazinyl; pyrazinyl; piperazinyl, triazinyl; morpholinyl, such as 1-, 2- and 3-morpholinyl; thiomorpholinyl, such as 1-, 2- and 3-thiomorpholinyl; pyranyl, such as 2-, 3- and 4-pyranyl; pyranonyl, such as pyran-4-on-2- or 3-yl; dihydropyranyl, such as 2,3-dihydropyran-2-, 3-, 4-, 5- and 6-yl; dihydropyranonyl, such as 2,3-dihydropyran-4-on-2-, 3-, 5- or 6-yl and 2,3-dihydropyran-6-on-2-, 3-, 4- or 5-yl; tetrahydropyranyl, such as 2-, 3- and 4-tetrahydropyranyl; tetrahydropyranonyl, such as tetrahydropyran-2-on-3-, 4-, 5- or 6-yl and tetrahydropyran-4-on-2- or 3-yl; and dioxanyl, such as 1,4-dioxan-2- or 3-yl or 1,3-dioxan-2- or 4-yl.

Preferably, the heterocyclic ring is unsubstituted or carries 1 or 2 substituents R^{x} selected from the group consisting of halogen, nitro, C₁-C₄-alkyl, C₁-C₄-haloalkyl, C₁-C₄-alkoxy and C₁-C₄-haloalkoxy and in particular from the group consisting of nitro and C₁-C₄-alkyl, especially nitro, methyl or ethyl.

In a preferred embodiment, the radical R^{4a} is a 5- or 6-membered heteroaromatic ring which contains a nitrogen atom and optionally one or two further heteroatoms selected from the group consisting of O, N and S as ring members and which may be partially or fully halogenated and/or carry 1, 2 or 3 radicals R^{x}, where R^{x} has the inventive or preferred meanings mentioned above (radical R^{4aa}).

Preferably, R^{4aa} is pyrrolyl, such as 1-, 2- and 3-pyrrolyl; pyrazolyl, such as 1-, 3-, 4- and 5-pyrazolyl; imidazolyl, such as 1-, 2-, 4- and 5-imidazolyl; triazolyl, such as 1- and 2-[1,3,5]-(1H)-triazolyl, 1-[1,2,3]-(1H)-triazolyl, 2-[1,2,3]-(2H)-triazolyl and also 1-, 3- and 5-[1,2,4]-(1H)-triazolyl; tetrazolyl, such as 1- and 5-[1,2,3,4]-(1H)-tetrazolyl; thiazolyl, such as 2-, 4- and 5-thiazolyl; isothiazolyl, such as 3-, 4- and 5-isothiazolyl; oxazolyl, such as 2-, 4- and 5-oxazolyl; isoxazolyl, such as 3-, 4- and 5-isoxazolyl; thiadiazolyl, such as 3- and 5-[1,2,4]-thiadiazolyl and also 2- and 5-[1,3,4]-thiadiazolyl; oxadiazolyl, such as 3- and 5-[1,2,4]-oxadiazolyl and also 2- and 5-[1,3,4]-oxadiazolyl; pyridyl, such as 2-, 3- and 4-pyridyl; pyrimidinyl, such as 2-, 4- and 5-pyrimidinyl; pyridazinyl, such as 2- and 3-pyridazinyl; pyrazinyl; or triazinyl, where the heteroaromatic rings may be partially or fully halogenated and/or carry 1, 2 or 3 radicals R^{x}, where R^{x} has the inventive or preferred meanings mentioned above or below. Particularly preferably, R^{4aa} is pyrazolyl, especially 1- and 3-pyrazolyl; triazolyl, especially 1- and 2-[1,2,4]-(1H)-triazolyl, 4-[1,2,4]-(4H)-triazolyl, 1-[1,2,3]-(1H)-triazolyl and 2-[1,2,3]-(2H)-triazolyl; thiazolyl, especially 2-thiazolyl; pyridyl, especially 2-pyridyl; pyridazinyl, especially 3-pyridazinyl; or pyrazinyl, and in particular pyrazolyl, especially 1- and 3-pyrazolyl; triazolyl, especially 1-[1,2,4]-(1H)-triazolyl, 4-[1,2,4]-(4H)-triazolyl, 1-[1,2,3]-(1H)-triazolyl and 2-[1,2,3]-(2H)-triazolyl; pyridazinyl, especially 3-pyridazinyl; or pyrazinyl, where the heteroaromatic rings may be partially or fully halogenated and/or carry 1, 2 or 3 radicals R^{x}, where R^{x} has the inventive or preferred meanings mentioned above or below. Particularly preferably, R^{4aa} is a 5-membered aromatic heterocycle which is attached via nitrogen and contains 1, 2 or 3, preferably 2 or 3, nitrogen atoms as ring members, especially 1-pyrazolyl, 1-[1,2,4]-(1H)-triazolyl, 4-[1,2,4]-(4H)-triazolyl, 1-[1,2,3]-(1H)-triazolyl and 2-[1,2,3]-(2H)-triazolyl.

R^{4aa} is preferably unsubstituted or carries 1 or 2 identical or different substituents R^{x}, which are as defined above or, preferably, selected from the group consisting of halogen, nitro, C₁-C₄-alkyl and C₁-C₄-haloalkyl and in particular from the group consisting of nitro and C₁-C₄-alkyl, especially methyl.

In an alternative preferred embodiment, the radical R^{4a} is a 5- or 6-membered saturated or partially unsaturated heterocyclic ring which contains a nitrogen atom and optionally one or two further heteroatoms selected from the group consisting of O, N and S and/or one or two carbonyl groups as ring members and which may be partially or fully halogenated and/or carry 1, 2 or 3 radicals R^{x}, where R^{x} has the inventive or preferred meanings mentioned above (radical R^{4ab}).

R^{4ab} is preferably a saturated heterocyclic radical selected from the group consisting of pyrrolidinyl, such as 1-, 2- and 3-pyrrolidinyl; pyrrolidonyl, such as 1-, 3-, 4- and 5-pyrrolidin-2-onyl and also 1-, 2-, 4- and 5-pyrrolidin-3-onyl; pyrrolidindionyl, such as 1-pyrrolidin-2,5-dionyl; pyrazolidinyl, such as 1-, 2-, 3- and 4-pyrazolidinyl; pyrazolidinonyl; imidazolidinyl, such as 1-, 2- and 4-imidazolidinyl; imidazolidinonyl, such as 1- and 4-imidazolidin-2-onyl and also 1-, 2-, 3- and 5-imidazolidin-4-onyl; thiazolidinyl, such as 2-, 4- and 5-thiazolidinyl; isothiazoidinlyl, such as 3-, 4- and 5-isothiazolidinyl; oxazolidinyl, such as 2-, 3-, 4- and 5-oxazolidinyl; oxazolidinonyl, such as 3-, 4- and 5-oxazolidin-2-onyl; isoxazolidinyl, such as 3-, 4- and 5-isoxazolidinyl; isoxazolidinonyl, such as 2-, 4- and 5-isoxazolidin-3-onyl; piperidyl, such as 1-, 2-, 3- and 4-piperidyl; morpholinyl, such as 1-, 2- and 3-morpholinyl; and thiomorpholinyl, such as 1-, 2- and 3-thiomorpholinyl. Alternatively, R^{4ab} is a partially unsaturated heterocyclic radical. Examples of partially unsaturated (non-aromatic) heterocycles are pyrrolinyl, such as 1-, 2- and 3-pyrrolinyl; pyrrolinonyl, pyrazolinyl, such as 1-, 3-, 4- and 5-pyrazolinyl; imidazolinyl, such as 1-, 2-, 4- and 5-imidazolinyl; thiazolinyl, such as 2-, 4- and 5-thiazolinyl; isothiazolinyl, such as 3-, 4- and 5-isothiazolinyl; oxazolinyl, such as 2-, 4- and 5-oxazolinyl; isoxazolinyl, such as 3-, 4- and 5-isoxazolinyl; dihydropyridyl, such as 1,4-dihydropyrid-1-, 2-, 3- and 4-yl; dihydropyridinonyl, such as 1-, 3-, 4-, 5- and 6-(1,2-dihydro)pyridin-2-onyl; tetrahydropyridyl, such as 1,2,3,6-tetrahydropyrid-1-, 2-, 3-, 4-, 5- and 6-yl and also 1,2,3,4-tetrahydropyrid-1-, 2-, 3-, 4-, 5- and 6-yl; and tetrahydropyridinonyl, such as 1-, 3-, 4-, 5- and 6-(1,2,3,4-tetrahydro)pyridin-2-onyl.

The heterocyclic radicals in R^{4ab} can be partially or fully halogenated and/or carry 1, 2 or 3 radicals R^{x}, where R^{x} has the inventive or preferred meanings mentioned above or below.

Particularly preferably, R^{4ab} is pyrrolidonyl, such as 1-, 3-, 4- and 5-pyrrolidin-2-onyl and also 1-, 2-, 4- and 5-pyrrolidin-3-onyl; imidazolidinonyl, such as 1- and 4-imidazolidin-2-onyl and also 1-, 2-, 3- and 5-imidazolidin-4-onyl; oxazolidinonyl, such as 3-, 4- and 5-oxazolidin-2-onyl; isoxazolidinonyl, such as 2-, 4- and 5-isoxazolidin-3-onyl; or dihydropyridinonyl, such as 1-, 3-, 4-, 5- and 6-(1,2-dihydro)pyridin-2-onyl, where the heterocyclic rings may be partially or fully halogenated and/or carry 1, 2 or 3 radicals R^{x}, where R^{x} has the inventive or preferred meanings given above or below.

R^{4ab} is preferably unsubstituted or carries 1 or 2 identical or different substituents R^{x} which are as defined above or, preferably, selected from the group consisting of halogen, C₁-C₄-alkyl and C₁-C₄-haloalkyl and in particular from the group consisting of C₁-C₄-alkyl, especially methyl.

Specifically, R^{4ab} is pyrrolidinonyl, especially pyrrolidin-2-on-1-yl, which is unsubstituted or carries 1 or 2 identical or different substituents R^{x} which are preferably selected from the group consisting of halogen, C₁-C₄-alkyl and C₁-C₄-haloalkyl and in particular from the group consisting of C₁-C₄-alkyl, especially methyl.

In an alternative preferred embodiment of the invention, R⁴ is CN or a radical R^{4b} of the formula —ON(═CR^{a}R^{b}), —NR^{c}N═CR^{a}R^{b}, —N═OR^{a}; —NR^{c}C(═W)—NR^{a}R^{b}, —NR^{a}C(═W)R^{c}, —NNR^{a}R^{b}C(═W)—X¹—R^{c}, —OC(═W)R^{c}, —O(C═W)NR^{a}R^{b}, —C(═W)R^{c}, —C₁—C(═W)NR^{a}R^{b}, —C(═W)NR^{a}OR^{b}, —CR^{a}R^{b}—C(═W)R^{c}, —C(═W)—NR^{a}—X²—R^{b}, —C(═NX²R^{a})—OR^{b} or —C(═NX²R^{a})—SR^{b}, where R^{a}, R^{b}, R^{c}, W, X¹ and X² are as defined above.

In the radical R^{4b}, R^{a}, R^{b}, R^{c}, R^{d}, R^{e} and R^{f} are preferably selected from the group consisting of H, hydroxyl, C₁-C₄-alkyl, C₁-C₄-alkylcarbonyl, C₁-C₄-alkoxy and phenyl, where phenyl may carry 1 or 2 substituents selected from the group consisting of halogen, C₁-C₄-alkyl, C₁-C₄-haloalkyl, C₁-C₄-alkoxy and C₁-C₄-haloalkoxy, where, if R^{a}, R^{b}, R^{c} or R^{d} are attached directly to an oxygen atom, they are not hydroxyl or C₁-C₄-alkoxy.

In the radical R^{4b}, R^{a}, R^{b} and R^{c} are particularly preferably selected from the group consisting of H, hydroxyl, C₁-C₄-alkyl, C₁-C₄-alkylcarbonyl, C₁-C₄-alkoxy and phenyl, where phenyl may carry 1 or 2 substituents selected from the group consisting of halogen, C₁-C₄-alkyl, C₁-C₄-haloalkyl, C₁-C₄-alkoxy and C₁-C₄-haloalkoxy, where, if R^{a}, R^{b} or R^{c} are attached directly to an oxygen atom, they are not hydroxyl or C₁-C₄-alkoxy, and R^{d}, R^{e} and R^{f} are selected from the group consisting of H and C₁-C₄-alkyl.

In the radical R^{4b}, X² is preferably a bond or —CO— and in particular a bond.

In this embodiment of the invention, R⁴ is particularly preferably CN or a radical R^{4ba} of the formula —NR^{a}C(═O)R^{c}, —C(═O)—R^{c}, —C(═O)—OR^{c}, —C(═NR^{d})R^{c}, —C(═NR^{d})—NR^{a}—X²—R^{b}, —C(═N—NR^{d}R^{e})—NR^{a}—X²—R^{b}, —C(═O)—NR^{a}—X²—R^{b} or —C(═S)—NR^{a}—X²—R^{b},

in which
X² is a single bond, —CO—, —CONH—, —COO—, —O— or —NR^{f}, where the left part of the divalent radicals is attached to the nitrogen atom;
R^{a} is hydrogen, hydroxyl, C₁-C₄-alkyl, C₁-C₄-alkoxy or C₁-C₄-alkylcarbonyl; and
R^{b}, R^{c}, R^{d}, R^{e} and R^{f} independently of one another are hydrogen, hydroxyl, C₁-C₄-alkyl, C₁-C₄-alkoxy or phenyl, where phenyl may carry 1 or 2 substituents selected from the group consisting of halogen, C₁-C₄-alkyl, C₁-C₄-haloalkyl, C₁-C₄-alkoxy and C₁-C₄-haloalkoxy,
   where, if R^{a}, R^{b}, R^{c} or R^{d} are attached directly to an oxygen atom, they are not hydroxyl or C₁-C₄-alkoxy.

In the radical R^{4ba}, R^{b} and R^{c} are particularly preferably selected from the group consisting of H, hydroxyl, C₁-C₄-alkyl, C₁-C₄-alkoxy and phenyl, where phenyl may carry 1 or 2 substituents selected from the group consisting of halogen, C₁-C₄-alkyl, C₁-C₄-haloalkyl, C₁-C₄-alkoxy and C₁-C₄-haloalkoxy and R^{a} is selected from the group consisting of H, hydroxyl, C₁-C₄-alkyl, C₁-C₄-alkylcarbonyl and C₁-C₄-alkoxy, where, if R^{a}, R^{b} or R^{c} are attached directly to an oxygen atom, they are not hydroxyl or C₁-C₄-alkoxy, and R^{d}, R^{e} and R^{f} are selected from the group consisting of H, C₁-C₄-alkyl and C₁-C₄-alkoxy.

In the radical R^{4ba}, X² is preferably a bond or —CO— and in particular a bond.

In an alternative preferred embodiment of the invention, R⁴ is a radical R^{4c} of the formula —NR^{a}R^{b}, —NR^{c}NR^{a}R^{b}, —NR^{a}—CN, —CR^{a}R^{b}—OR^{c}, —CR^{a}R^{b}—SR^{c} or —CR^{a}R^{b}—NR^{c}R^{d}, where R^{a}, R^{b}, R^{c} and R^{d} are as defined above. R^{a}, R^{b}, R^{c} and R^{d} are preferably independently H, C₁-C₄-alkyl or C₁-C₄-alkoxy and in particular H or C₁-C₄-alkyl.

In an alternative preferred embodiment of the invention, R⁴ is a radical R^{4d} of the formula

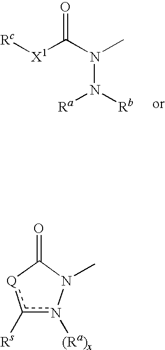

in which
x is 0 or 1;
X¹ and X¹¹ are independently oxygen or N—R^{f};
Q is C(H)—R^{t}, C—R^{t}, N—N(H)—R^{f} or N—R^{f};
[symbol] is a single bond or a double bond;
R^{a}, R^{b}, R^{c}, R^{f} independently of one another are hydrogen, C₁-C₆-alkyl, C₂-C₈-alkenyl, C₂-C₈-alkynyl, C₃-C₆-cycloalkyl or C₄-C₆-cycloalkenyl, or
R^{a} and R^{b} together with the nitrogen atom to which they are attached form a group R^{c}—X¹¹—C(R⁹)═N;
R^{g}, R^{s} and R^{t} are defined like R^{c} and may furthermore be halogen or cyano; or
R^{s} together with the carbon atom to which it is attached forms a CO group;
   where the aliphatic, alicyclic or aromatic groups in the radicals R^{a}, R^{b}, R^{c}, R^{f}, R^{g}, R^{s} and/or R^{t} may be partially or fully halogenated and/or may carry 1, 2, 3 or 4 substituents R^{v}, where
   R^{v} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy or C₃-C₆-cycloalkenyloxy, or two of the radicals R^{a}, R^{b}, R^{c} or R^{d} together with the atoms to which they are attached form a 5- or 6-membered saturated, partially unsaturated or aromatic heterocycle having 1, 2, 3 or 4 heteroatoms selected from the group consisting of N, O and S.

In an alternative preferred embodiment of the invention, R⁴ is a radical R^{4e} of the formula

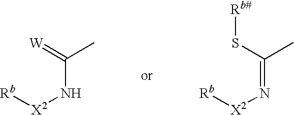

in which
X² is a single bond, —CO—, —CONH—, —COO—, —O— or —NR^{f}—, where the left part of the divalent radicals is attached to the nitrogen atom;
R^{f} is hydrogen, methyl or C₁-C₄-alkylcarbonyl;
R^{b} is hydrogen, methyl, benzyl, trifluoromethyl, allyl, propargyl or methoxymethyl;
R^{b#}, R^{d#} are independently hydrogen, C₁-C₆-alkyl or C₂-C₆-alkynyl;
W is S or NR^{d#};
where the aliphatic groups in the radicals R^{b}, R^{b#}, R^{d} and/or R^{f} may carry one or two substituents R^{w}; where
R^{w} is halogen, OR^{z}, NHR^{z}, C₁-C₆-alkyl, C₁-C₄-alkoxycarbonyl, C₁-C₄-acylamino, [1,3]dioxolane-C₁-C₄-alkyl or [1,3]dioxane-C₁-C₄-alkyl in which R^{z} is hydrogen, methyl, allyl or propargyl.

A particular embodiment of the invention relates to compounds of the formula 1.1

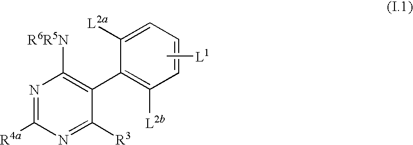

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4a} has the meanings given above and is preferably a radical R^{4aa} or R^{4ab}; and
R⁵ and R⁶ have the meanings given above, preferably the meanings given as being preferred.

A further particular embodiment of the invention relates to compounds of the formula I.2

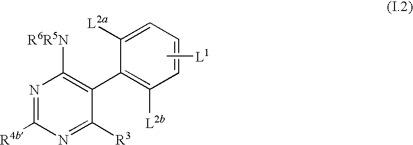

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4b′} is CN or a radical R^{4b} which has the general or preferably preferred meanings given above and is preferably CN or a radical R^{4ba}; and
R⁵ and R⁶ have the meanings given above, preferably the meanings given as being preferred.

A further particular embodiment of the invention relates to compounds of the formula I.3

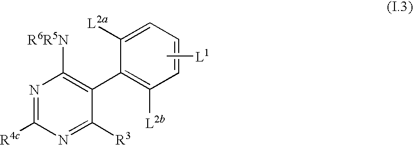

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4c} has the general or preferably preferred meanings given above; and
R⁵ and R⁶ have the meanings given above, preferably the meanings given as being preferred.

A further particular embodiment of the invention relates to compounds of the formula I.4

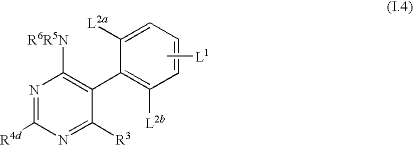

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4d} has the general or preferably preferred meanings given above; and
R⁵ and R⁶ have the meanings given above, preferably the meanings given as being preferred.

A further particular embodiment of the invention relates to compounds of the formula I.5

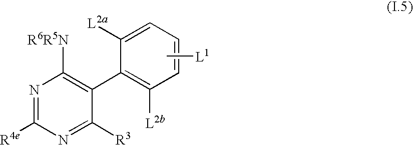

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4e} has the general or preferably preferred meanings given above; and
R⁵ and R⁶ have the meanings given above, preferably the meanings given as being preferred.

A further particular embodiment of the invention relates to compounds of the formula I.6

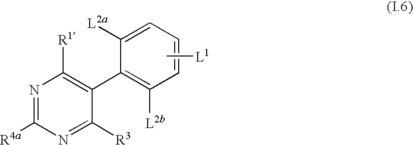

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and
R^{4a} has the meanings given above and is preferably a radical R^{4aa} or R^{4ab}.

A further particular embodiment of the invention relates to compounds of the formula I.7

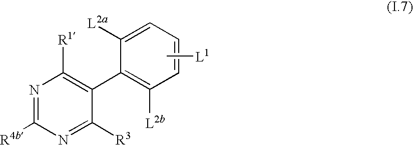

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and
R^{4b′} is CN or a radical R^{4b} which has the general or preferably preferred meanings given above and is preferably CN or a radical R^{4ba}.

A further particular embodiment of the invention relates to compounds of the formula I.8

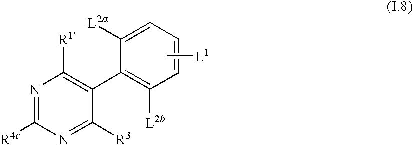

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and
R^{4c} has the general or preferably preferred meanings given above.

A further particular embodiment of the invention relates to compounds of the formula I.9

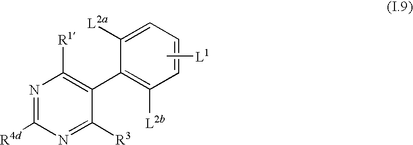

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and
R^{4d} has the general or preferably preferred meanings given above.

A further particular embodiment of the invention relates to compounds of the formula I.10

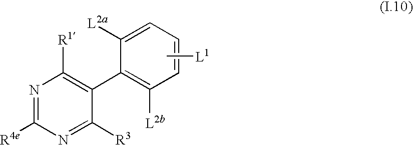

in which
L¹ is as defined above and is preferably a radical L¹¹, L¹² or L¹³ and in particular a radical L¹¹ or L¹²;
L^{2a}, L^{2b} independently of one another are H, halogen, C₁-C₄-alkyl, C₁-C₄-haloalkyl, C₁-C₄-alkoxy or C₁-C₄-haloalkoxy and preferably H, halogen or C₁-C₄-alkyl, where preferably at least one of the radicals is not H;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and
R^{4e} has the general or preferably preferred meanings given above.

A further particular embodiment of the invention relates to compounds of the formula I.11

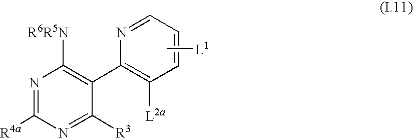

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl and in particular halogen or C₁-C₄-alkyl;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4a} has the meanings given above and is preferably a radical R^{4aa} or R^{4ab}; and
R⁵ and R⁶ have the meanings given above, preferably the meanings given as being preferred.

A further particular embodiment of the invention relates to compounds of the formula I.12

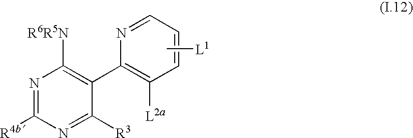

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl and in particular halogen or C₁-C₄-alkyl;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4b′} is CN or a radical R^{4b} which has the general or preferably preferred meanings given above and is preferably CN or a radical R^{4ba}; and
R⁵ and R⁶ have the meanings given above, preferably the meanings given as being preferred.

A further particular embodiment of the invention relates to compounds of the formula I.13

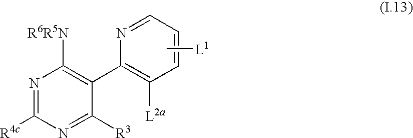

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl and in particular halogen or C₁-C₄-alkyl;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4c} has the general or preferably preferred meanings given above; and
R⁵ and R⁶ have the meanings given above, preferably the meanings given as being preferred.

A further particular embodiment of the invention relates to compounds of the formula I.14

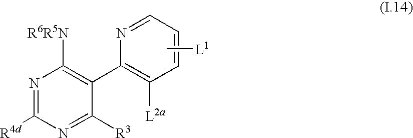

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl and in particular halogen or C₁-C₄-alkyl;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4d} has the general or preferably preferred meanings given above; and
R⁵ and R⁶ have the meanings given above, preferably the meanings given as being preferred.

A further particular embodiment of the invention relates to compounds of the formula I.15

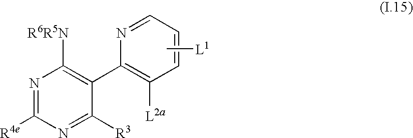

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl and in particular halogen or C₁-C₄-alkyl;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen;
R^{4e} has the general or preferably preferred meanings given above; and
R⁵ and R⁶ have the meanings given above, preferably the meanings given as being preferred.

A further particular embodiment of the invention relates to compounds of the formula I.16

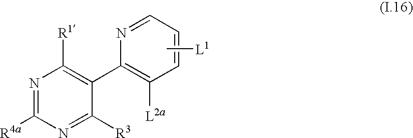

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl, and in particular halogen or C₁-C₄-alkyl;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and

R^{4a} has the meanings given above and is preferably a radical R^{4aa} or R^{4ab}.

A further particular embodiment of the invention relates to compounds of the formula I.17

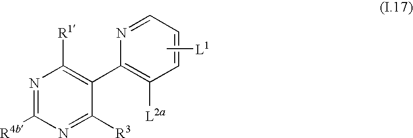

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl, and in particular halogen or C₁-C₄-alkyl;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and
R^{4b′} is CN or a radical R^{4b} which has the general or preferably preferred meanings given above and is preferably CN or a radical R^{4ba}.

A further particular embodiment of the invention relates to compounds of the formula I.18

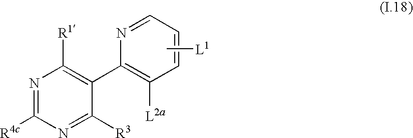

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl, and in particular halogen or C₁-C₄-alkyl;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and
R^{4c} has the general or preferably preferred meanings given above.

A further particular embodiment of the invention relates to compounds of the formula I.19

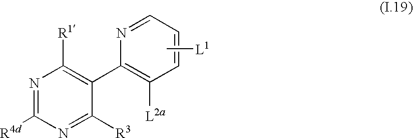

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl, and in particular halogen or C₁-C₄-alkyl;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and
R^{4d} has the general or preferably preferred meanings given above.

A further particular embodiment of the invention relates to compounds of the formula I.20

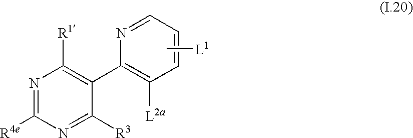

in which
L¹ is as defined above and is preferably a radical L¹¹, L¹² or L¹³ and in particular a radical L¹¹ or L¹²;
L^{2a} is H, halogen, C₁-C₄-alkyl, C₁-C₄-haloalkyl, C₁-C₄-alkoxy or C₁-C₄-haloalkoxy, preferably H, halogen or C₁-C₄-alkyl, and in particular halogen or C₁-C₄-alkyl;
R^{1′} has the meanings given above, preferably the meanings given as being preferred;
R³ is halogen, C₁-C₄-alkyl, C₁-C₄-haloalkyl, C₁-C₄-alkoxy, C₁-C₄-haloalkoxy or cyano, preferably halogen, C₁-C₄-alkyl or cyano, in particular halogen; and
R^{4e} has the general or preferably preferred meanings given above.

In compounds I. 1 to I. 10, the substituent L¹ is preferably attached in the 3- or, in particular, 4-position, based on the point of attachment of the phenyl ring to the pyrimidine ring in the 1-position, i.e. L¹ is preferably in the meta- or, in particular, paraposition in relation to the point of attachment to the pyrimidine ring.

In compounds I. 11 to I. 20, the radical L¹ is preferably attached in the 4-, 5- or 6-position, in particular in the 5-position, based on the point of attachment of the pyridyl ring to the pyrimidine ring in the 2-position.

Examples of preferred compounds of the general formula I are those of the formulae I.a, I.b, I.c and I.d

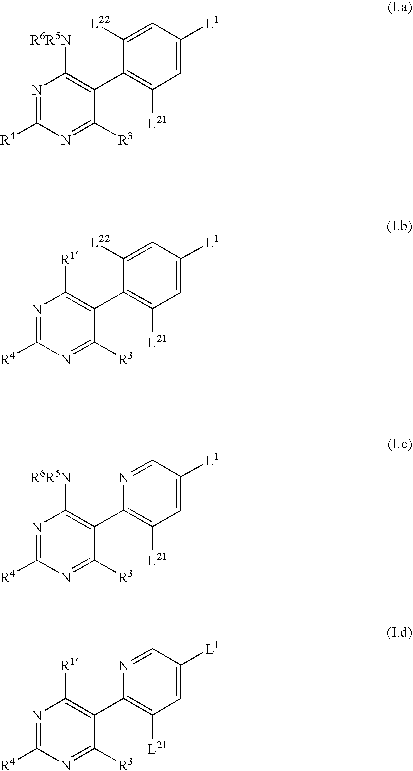

in which the variables R^{1′}, R³, R⁴, R⁵, R⁶ and L¹ have the general or preferred meanings given above and L²¹ and L²² are H or have one of the general or preferred meanings given for L².

Examples of particularly preferred compounds of the general formula I are the compounds I compiled in Tables 1 to 41160 below. Moreover, the meanings mentioned for the individual variables in the tables are per se, independently of the combination in which they are mentioned, a particularly preferred embodiment of the substituents in question.

### Table 1

Compounds of the formula I.a in which R³ is chlorine, L²¹ is H, L²² is H, L¹ is —(OCH₂)₂—OH, R⁴ is pyrazol-1-yl and the combination of R⁵ and R⁶ for a compound corresponds in each case to one row of Table A.

### Table 2

Compounds of the formula I.a in which R³ is chlorine, L²¹ is H, L²² is H, L¹ is —(OCH₂)₂—OH, R⁴ is 3-methylpyrazol-1-yl and the combination of R⁵ and R⁶ for a compound corresponds in each case to one row of Table A.

### Table 3

Compounds of the formula I.a in which R³ is chlorine, L²¹ is H, L²² is H, L¹ is —(OCH₂)₂—OH, R⁴ is [1,2,3]-(1H)-triazol-1-yl and the combination of R⁵ and R⁶ for a compound corresponds in each case to one row of Table A.

### Table 4

Compounds of the formula I.a in which R³ is chlorine, L²¹ is H, L²² is H, L¹ is —(OCH₂)₂—OH, R⁴ is [1,2,3]-(2H)-triazol-2-yl and the combination of R⁵ and R⁶ for a compound corresponds in each case to one row of Table A.

### Table 5

Compounds of the formula I.a in which R³ is chlorine, L²¹ is H, L²² is H, L¹ is —(OCH₂)₂—OH, R⁴ is [1,2,4]-(1H)-triazol-1-yl and the combination of R⁵ and R⁶ for a compound corresponds in each case to one row of Table A.

### Table 6

Compounds of the formula I.a in which R³ is chlorine, L²¹ is H, L²² is H, L¹ is —(OCH₂)₂—OH, R⁴ is [1,2,4]-(4H)-triazol-4-yl and the combination of R⁵ and R⁶ for a compound corresponds in each case to one row of Table A.

### Table 7

Compounds of the formula I.a in which R³ is chlorine, L²¹ is H, L²² is H, L¹ is —(OCH₂)₂—OH, R⁴ is 3-nitro-[1,2,4]-(1H)-triazol-1-yl and the combination of R⁵ and R⁶ for a compound corresponds in each case to one row of Table A.

### Table 8

Compounds of the formula I.a in which R³ is chlorine, L²¹ is H, L²² is H, L¹ is —(OCH₂)₂—OH, R⁴ is thiazol-2-yl and the combination of R⁵ and R⁶ for a compound corresponds in each case to one row of Table A.

### Table 9

Compounds of the formula I.a in which R³ is chlorine, L²¹ is H, L²² is H, L¹ is —(OCH₂)₂—OH, R⁴ is pyridin-2-yl and the combination of R⁵ and R⁶ for a compound corresponds in each case to one row of Table A.

### Table 10

Compounds of the formula I.a in which R³ is chlorine, L²¹ is H, L²² is H, L¹ is —(OCH₂)₂—OH, R⁴ is 4-methylpyridin-2-yl and the combination of R⁵ and R⁶ for a compound corresponds in each case to one row of Table A.

### Table 11

Compounds of the formula I.a in which R³ is chlorine, L²¹ is H, L²² is H, L¹ is —(OCH₂)₂—OH, R⁴ is 6-methylpyridin-2-yl and the combination of R⁵ and R⁶ for a compound corresponds in each case to one row of Table A.

### Table 12

Compounds of the formula I.a in which R³ is chlorine, L²¹ is H, L²² is H, L¹ is —(OCH₂)₂—OH, R⁴ is pyridazin-3-yl and the combination of R⁵ and R⁶ for a compound corresponds in each case to one row of Table A.

### Table 13

Compounds of the formula I.a in which R³ is chlorine, L²¹ is H, L²² is H, L¹ is —(OCH₂)₂—OH, R⁴ is pyrazin-2-yl and the combination of R⁵ and R⁶ for a compound corresponds in each case to one row of Table A.

### Table 14

Compounds of the formula I.a in which R³ is chlorine, L²¹ is H, L²² is H, L¹ is —(OCH₂)₂—OH, R⁴ is pyrrolidin-2-on-1-yl and the combination of R⁵ and R⁶ for a compound corresponds in each case to one row of Table A.

### Table 15

Compounds of the formula I.a in which R³ is chlorine, L²¹ is H, L²² is H, L¹ is —(OCH₂)₂—OH, R⁴ is 5-methylpyrrolidin-2-on-1-yl and the combination of R⁵ and R⁶ for a compound corresponds in each case to one row of Table A.

### Table 16

Compounds of the formula I.a in which R³ is chlorine, L²¹ is H, L²² is H, L¹ is —(OCH₂)₂—OH, R⁴ is —C(═O)-(p-tolyl) and the combination of R⁵ and R⁶ for a compound corresponds in each case to one row of Table A.

### Table 17

Compounds of the formula I.a in which R³ is chlorine, L²¹ is H, L²² is H, L¹ is —(OCH₂)₂—OH, R⁴ is —C(═O)OCH₃ and the combination of R⁵ and R⁶ for a compound corresponds in each case to one row of Table A.

### Table 18

Compounds of the formula I.a in which R³ is chlorine, L²¹ is H, L²² is H, L¹ is —(OCH₂)₂—OH, R⁴ is —C(═O)OCH(CH₃)₂ and the combination of R⁵ and R⁶ for a compound corresponds in each case to one row of Table A.

### Table 19

Compounds of the formula I.a in which R³ is chlorine, L²¹ is H, L²² is H, L¹ is —(OCH₂)₂—OH, R⁴ is —C(═O)NH₂ and the combination of R⁵ and R⁶ for a compound corresponds in each case to one row of Table A.

### Table 20

Compounds of the formula I.a in which R³ is chlorine, L²¹ is H, L²² is H, L¹ is —(OCH₂)₂—OH, R⁴ is —C(═O)NHCH₃ and the combination of R⁵ and R⁶ for a compound corresponds in each case to one row of Table A.

### Table 21

Compounds of the formula I.a in which R³ is chlorine, L²¹ is H, L²² is H, L¹ is —(OCH₂)₂—OH, R⁴ is —C(═O)NH—C(═O)CH₃ and the combination of R⁵ and R⁶ for a compound corresponds in each case to one row of Table A.

### Table 22

Compounds of the formula I.a in which R³ is chlorine, L²¹ is H, L²² is H, L¹ is —(OCH₂)₂—OH, R⁴ is —C(═S)NH₂ and the combination of R⁵ and R⁶ for a compound corresponds in each case to one row of Table A.

### Table 23

Compounds of the formula I.a in which R³ is chlorine, L²¹ is H, L²² is H, L¹ is —(OCH₂)₂—OH, R⁴ is —C(═NOH)OH and the combination of R⁵ and R⁶ for a compound corresponds in each case to one row of Table A.

### Table 24

Compounds of the formula I.a in which R³ is chlorine, L²¹ is H, L²² is H, L¹ is —(OCH₂)₂—OH, R⁴ is —C(═NOH)CH₃ and the combination of R⁵ and R⁶ for a compound corresponds in each case to one row of Table A.

### Table 25

Compounds of the formula I.a in which R³ is chlorine, L²¹ is H, L²² is H, L¹ is —(OCH₂)₂—OH, R⁴ is —C(═NOH)NH₂ and the combination of R⁵ and R⁶ for a compound corresponds in each case to one row of Table A.

### Table 26

Compounds of the formula I.a in which R³ is chlorine, L²¹ is H, L²² is H, L¹ is —(OCH₂)₂—OH, R⁴ is —C(═NOCH₃)CH₃ and the combination of R⁵ and R⁶ for a compound corresponds in each case to one row of Table A.

### Table 27

Compounds of the formula I.a in which R³ is chlorine, L²¹ is H, L²² is H, L¹ is —(OCH₂)₂—OH, R⁴ is —C(═NOCH₃)NH₂ and the combination of R⁵ and R⁶ for a compound corresponds in each case to one row of Table A.

### Table 28

Compounds of the formula I.a in which R³ is chlorine, L²¹ is H, L²² is H, L¹ is —(OCH₂)₂—OH, R⁴ is —C(═NH)NH—C(═O)CH₃ and the combination of R⁵ and R⁶ for a compound corresponds in each case to one row of Table A.

### Table 29

Compounds of the formula I.a in which R³ is chlorine, L²¹ is H, L²² is H, L¹ is —(OCH₂)₂—OH, R⁴ is —C(═N—N(CH₃)₂)NH₂ and the combination of R⁵ and R⁶ for a compound corresponds in each case to one row of Table A.

### Table 30

Compounds of the formula I.a in which R³ is chlorine, L²¹ is H, L²² is H, L¹ is —(OCH₂)₂—OH, R⁴ is —N(CH₃)(C(═O)CH₃) and the combination of R⁵ and R⁶ for a compound corresponds in each case to one row of Table A.

### Table 31

Compounds of the formula I.a in which R³ is chlorine, L²¹ is H, L²² is H, L¹ is —(OCH₂)₂—OH, R⁴ is —N(H)(C(═O)OCH₃) and the combination of R⁵ and R⁶ for a compound corresponds in each case to one row of Table A.

### Table 32

Compounds of the formula I.a in which R³ is chlorine, L²¹ is H, L²² is H, L¹ is —(OCH₂)₂—OH, R⁴ is —N(CH₃)(C(═O)OCH₃) and the combination of R⁵ and R⁶ for a compound corresponds in each case to one row of Table A.

### Table 33

Compounds of the formula I.a in which R³ is chlorine, L²¹ is H, L²² is H, L¹ is —(OCH₂)₂—OH, R⁴ is —N(OCH₃)(C(═O)OCH₃) and the combination of R⁵ and R⁶ for a compound corresponds in each case to one row of Table A.

### Table 34

Compounds of the formula I.a in which R³ is chlorine, L²¹ is H, L²² is H, L¹ is —(OCH₂)₂—OH, R⁴ is —N(H)(CN) and the combination of R⁵ and R⁶ for a compound corresponds in each case to one row of Table A.

### Table 35

Compounds of the formula I.a in which R³ is chlorine, L²¹ is H, L²² is H, L¹ is —(OCH₂)₂—OH, R⁴ is —N(CH₃)(CN) and the combination of R⁵ and R⁶ for a compound corresponds in each case to one row of Table A.

### Tables 36 to 70

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —(OCH₂)₂—OCH₃ instead of —(OCH₂)₂—OH.

### Tables 71 to 105

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —(OCH₂)₂—OC₂H₅ instead of —(OCH₂)₂—OH.

### Tables 106 to 140

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —(OCH₂)₃—OH instead of —(OCH₂)₂—OH.

### Tables 141 to 175

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —(OCH₂)₃—OCH₃ instead of —(OCH₂)₂—OH.

### Tables 176 to 210

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —(OCH₂)₃—OC₂H₅ instead of —(OCH₂)₂—OH.

### Tables 211 to 245

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂—OH instead of —(OCH₂)₂—OH.

### Tables 246 to 280

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂—OCH₃ instead of —(OCH₂)₂—OH.

### Tables 281 to 315

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂—OC₂H₅ instead of —(OCH₂)₂—OH.

### Tables 316 to 350

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —(O—CH₂CH₂)₂—OH instead of —(OCH₂)₂—OH.

### Tables 351 to 385

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —(O—CH₂CH₂)₂—OCH₃ instead of —(OCH₂)₂—OH.

### Tables 386 to 420

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —(O—CH₂CH₂)₂—OC₂H₅ instead of —(OCH₂)₂—OH.

### Tables 421 to 455

Compounds of the formula I.a in which the combination of R³, R⁴, L²¹ and L²² is as defined in any of Tables 1 to 35, the combination of R⁵ and R⁵ for a compound corresponds in each case to one row of Table A and L¹ is —O—CH₂CH₂—NHCH₃ instead of —(OCH₂)₂—OH.

### Tables 456 to 490

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂—N(CH₃)₂ instead of —(OCH₂)₂—OH.

### Tables 491 to 525

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂—NHC₂H₅ instead of —(OCH₂)₂—OH.

### Tables 526 to 560

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂—N(C₂H₅)₂ instead of —(OCH₂)₂—OH.

### Tables 561 to 595

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—NHCH₃ instead of —(OCH₂)₂—OH.

### Tables 596 to 630

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—N(CH₃)₂ instead of —(OCH₂)₂—OH.

### Tables 631 to 665

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—NHC₂H₅ instead of —(OCH₂)₂—OH.

### Tables 666 to 700

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—N(C₂H₅)₂ instead of —(OCH₂)₂—OH.

### Tables 701 to 735

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂CH₂—NHCH₃ instead of —(OCH₂)₂—OH.

### Tables 736 to 770

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂CH₂—N(CH₃)₂ instead of —(OCH₂)₂—OH.

### Tables 771 to 805

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂CH₂—NHC₂H₅ instead of —(OCH₂)₂—OH.

### Tables 806 to 840

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂CH₂—N(C₂H₅)₂ instead of —(OCH₂)₂—OH.

### Tables 841 to 875

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—OH instead of —(OCH₂)₂—OH.

### Tables 876 to 910

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—OCH₃ instead of —(OCH₂)₂—OH.

### Tables 911 to 945

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—OCH₂CH₃ instead of —(OCH₂)₂—OH.

### Tables 946 to 980

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—O-(pyridin-2-yl) instead of —(OCH₂)₂—OH.

### Tables 981 to 1015

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂-Q-(pyrimidin-4-yl) instead of —(OCH₂)₂—OH.

### Tables 1016 to 1050

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—O-(4-chloro-pyrimidin-6-yl) instead of —(OCH₂)₂—OH.

### Tables 1051 to 1085

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂— (pyrazol-1-yl) instead of —(OCH₂)₂—OH.

### Tables 1086 to 1120

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂CH₂-([1,2,3]-(1H)-triazol-1-yl) instead of —(OCH₂)₂—OH.

### Tables 1121 to 1155

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂-([1,2,4]-(1H)-triazol-1-yl) instead of —(OCH₂)₂—OH.

### Tables 1156 to 1190

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂-([1,2,3]-(2H)-triazol-2-yl) instead of —(OCH₂)₂—OH.

### Tables 1191 to 1225

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂— (pyrrolidin-2-on-1-yl) instead of —(OCH₂)₂—OH.

### Tables 1226 to 1260

Compounds of the formula I.a in which the combination of R³, R⁴, L²¹ und L²² is as defined in any of Tables 1 to 35, the combination of R⁵ and R⁶ for a compound corresponds in each case to one row of Table A and L¹ is —O—CH₂CH₂CH₂—Cl instead of —(OCH₂)₂—OH.

### Tables 1261 to 1295

Compounds of the formula I.a in which the combination of R³, R⁴, L²′ and L²² is as defined in any of Tables 1 to 35, the combination of R⁵ and R⁶ for a compound corresponds in each case to one row of Table A and L¹ is —O—CH₂CH₂CH₂—NH—C(═O)—O—CH₃.

### Tables 1296 to 1330

Compounds of the formula I.a in which the combination of R³, R⁴, L²′ and L²² is as defined in any of Tables 1 to 35, the combination of R⁵ and R⁶ for a compound corresponds in each case to one row of Table A and L¹ is —O—CH₂CH₂CH₂—NH—C(═O)—O—CH₂CH₃.

### Tables 1331 to 1365

Compounds of the formula I.a in which the combination of R³, R⁴, L²′ and L²² is as defined in any of Tables 1 to 35, the combination of R⁵ and R⁶ for a compound corresponds in each case to one row of Table A and L¹ is —O—CH₂CH₂CH₂—NH—C(═O)—O—C(CH₃)₃.

### Tables 1366 to 1400

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—NCH₃—C(═O)—O—CH₃.

### Tables 1401 to 1435

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—NCH₃—C(═O)—O—CH₂CH₃.

### Tables 1436 to 1470

Compounds of the formula I.a in which the combination of R³, R⁴, L²¹ and L²² is as defined in any of Tables 1 to 35, the combination of R⁵ and R⁶ for a compound corresponds in each case to one row of Table A and L¹ is —O—CH₂CH₂CH₂—NCH₃—C(═O)—O—C(CH₃)₃.

### Tables 1471 to 2940

Compounds of the formula I.a in which the combination of R³, R⁴ and L¹ is as defined in any of Tables 1 to 1470, the combination of R⁵ and R⁶ for a compound corresponds in each case to one row of Table A, L²¹ is fluorine and L²² is H.

### Tables 2941 to 4410

Compounds of the formula I.a in which the combination of R³, R⁴ and L¹ is as defined in any of Tables 1 to 1470, the combination of R⁵ and R⁶ for a compound corresponds in each case to one row of Table A, L²¹ is chlorine and L²² is H.

### Tables 4411 to 5880

Compounds of the formula I.a in which the combination of R³, R⁴ and L¹ is as defined in any of Tables 1 to 1470, the combination of R⁵ and R⁶ for a compound corresponds in each case to one row of Table A, L²¹ is methyl and L²² is H.

### Tables 5881 to 7350

Compounds of the formula I.a in which the combination of R³, R⁴ and L¹ is as defined in any of Tables 1 to 1470, the combination of R⁵ and R⁶ for a compound corresponds in each case to one row of Table A, L²¹ is fluorine and L²² is fluorine.

### Tables 7351 to 8820

Compounds of the formula I.a in which the combination of R³, R⁴ and L¹ is as defined in any of Tables 1 to 1470, the combination of R⁵ and R⁶ for a compound corresponds in each case to one row of Table A, L²¹ is chlorine and L²² is fluorine.

### Tables 8821 to 10290

Compounds of the formula I.a in which the combination of R³, R⁴ and L¹ is as defined in any of Tables 1 to 1470, the combination of R⁵ and R⁶ for a compound corresponds in each case to one row of Table A, L²¹ is methyl and L²² is fluorine.

### Tables 10291 to 11760

Compounds of the formula I.a in which the combination of R³, R⁴ and L¹ is as defined in any of Tables 1 to 1470, the combination of R⁵ and R⁶ for a compound corresponds in each case to one row of Table A, L²¹ is chlorine and L²² is chlorine.

### Tables 11761 to 13230

Compounds of the formula I.a in which the combination of R³, R⁴ and L¹ is as defined in any of Tables 1 to 1470, the combination of R⁵ and R⁶ for a compound corresponds in each case to one row of Table A, L²¹ is methyl and L²² is chlorine.

### Tables 13231 to 14700

Compounds of the formula I.a in which the combination of R³, R⁴ and L¹ is as defined in any of Tables 1 to 1470, the combination of R⁵ and R⁶ for a compound corresponds in each case to one row of Table A, L²¹ is methyl and L²² is methyl.

### Tables 14701 to 29400

Compounds of the formula I.b in which the combination of R³, R⁴; L¹, L²¹ and L²² is as defined in any of Tables 1 to 14700 and R^{1′} for a compound corresponds in each case to one row of Table B.

### Tables 29401 to 35280

Compounds of the formula I.c in which the combination of R³, R⁴; L¹ and L²¹ is as defined in any of Tables 1 to 5880 and the combination of R⁵ and R⁶ for a compound corresponds in each case to one row of Table A.

### Tables 35281 to 41160

Compounds of the formula I.d in which the combination of R³, R⁴; L¹ and L²¹ is as defined in any of Tables 1 to 5880 and R^{1′} for a compound corresponds in each case to one row of Table B.

TABLE A

| No. | R⁵ | R⁶ |
|---|---|---|
| A-1 | H | H |
| A-2 | CH₃ | H |
| A-3 | CH₃ | CH₃ |
| A-4 | CH₂CH₃ | H |
| A-5 | CH₂CH₃ | CH₃ |
| A-6 | CH₂CH₃ | CH₂CH₃ |
| A-7 | CH₂CHF₂ | H |
| A-8 | CH₂CHF₂ | CH₃ |
| A-9 | CH₂CHF₂ | CH₂CH₃ |
| A-10 | CH₂CF₃ | H |
| A-11 | CH₂CF₃ | CH₃ |
| A-12 | CH₂CF₃ | CH₂CH₃ |
| A-13 | CH₂CCl₃ | H |
| A-14 | CH₂CCl₃ | CH₃ |
| A-15 | CH₂CCl₃ | CH₂CH₃ |
| A-16 | CH₂CH₂CH₃ | H |
| A-17 | CH₂CH₂CH₃ | CH₃ |
| A-18 | CH₂CH₂CH₃ | CH₂CH₃ |
| A-19 | CH₂CH₂CH₃ | CH₂CH₂CH₃ |
| A-20 | CH(CH₃)₂ | H |
| A-21 | CH(CH₃)₂ | CH₃ |
| A-22 | CH(CH₃)₂ | CH₂CH₃ |
| A-23 | CH(CH₃)₂ | CH₂CH₂CH₃ |
| A-24 | CH₂CH(CH₃)₂ | H |
| A-25 | CH₂CH(CH₃)₂ | CH₃ |
| A-26 | CH₂CH(CH₃)₂ | CH₂CH₃ |
| A-27 | CH₂CH(CH₃)₂ | CH₂CH₂CH₃ |
| A-28 | CH₂CH₂CH₂CH₃ | H |
| A-29 | CH₂CH₂CH₂CH₃ | CH₃ |
| A-30 | CH₂CH₂CH₂CH₃ | CH₂CH₃ |
| A-31 | CH₂CH₂CH₂CH₃ | CH₂CH₂CH₃ |
| A-32 | CH₂CH₂CH₂CH₃ | CH₂CH₂CH₂CH₃ |
| A-33 | (±)CH(CH₃)—CH₂CH₃ | H |
| A-34 | (±)CH(CH₃)—CH₂CH₃ | CH₃ |
| A-35 | (±)CH(CH₃)—CH₂CH₃ | CH₂CH₃ |
| A-36 | (±)CH(CH₃)—CH₂CH₃ | CH₂CH₂CH₃ |
| A-37 | (S)CH(CH₃)—CH₂CH₃ | H |
| A-38 | (S)CH(CH₃)—CH₂CH₃ | CH₃ |
| A-39 | (S)CH(CH₃)—CH₂CH₃ | CH₂CH₃ |
| A-40 | (S)CH(CH₃)—CH₂CH₃ | CH₂CH₂CH₃ |
| A-41 | (R)CH(CH₃)—CH₂CH₃ | H |
| A-42 | (R)CH(CH₃)—CH₂CH₃ | CH₃ |
| A-43 | (R)CH(CH₃)—CH₂CH₃ | CH₂CH₃ |
| A-44 | (R)CH(CH₃)—CH₂CH₃ | CH₂CH₂CH₃ |
| A-45 | (±)CH(CH₃)—CH(CH₃)₂ | H |
| A-46 | (±)CH(CH₃)—CH(CH₃)₂ | CH₃ |
| A-47 | (±)CH(CH₃)—CH(CH₃)₂ | CH₂CH₃ |
| A-48 | (±)CH(CH₃)—CH(CH₃)₂ | CH₂CH₂CH₃ |
| A-49 | (S)CH(CH₃)—CH(CH₃)₂ | H |
| A-50 | (S)CH(CH₃)—CH(CH₃)₂ | CH₃ |
| A-51 | (S)CH(CH₃)—CH(CH₃)₂ | CH₂CH₃ |
| A-52 | (S)CH(CH₃)—CH(CH₃)₂ | CH₂CH₂CH₃ |
| A-53 | (R)CH(CH₃)—CH(CH₃)₂ | H |
| A-54 | (R)CH(CH₃)—CH(CH₃)₂ | CH₃ |
| A-55 | (R)CH(CH₃)—CH(CH₃)₂ | CH₂CH₃ |
| A-56 | (R)CH(CH₃)—CH(CH₃)₂ | CH₂CH₂CH₃ |
| A-57 | (±)CH(CH₃)—C(CH₃)₃ | H |
| A-58 | (±)CH(CH₃)—C(CH₃)₃ | CH₃ |
| A-59 | (±)CH(CH₃)—C(CH₃)₃ | CH₂CH₃ |
| A-60 | (±)CH(CH₃)—C(CH₃)₃ | CH₂CH₂CH₃ |
| A-61 | (S)CH(CH₃)—C(CH₃)₃ | H |
| A-62 | (S)CH(CH₃)—C(CH₃)₃ | CH₃ |
| A-63 | (S)CH(CH₃)—C(CH₃)₃ | CH₂CH₃ |
| A-64 | (S)CH(CH₃)—C(CH₃)₃ | CH₂CH₂CH₃ |
| A-65 | (R)CH(CH₃)—C(CH₃)₃ | H |
| A-66 | (R)CH(CH₃)—C(CH₃)₃ | CH₃ |
| A-67 | (R)CH(CH₃)—C(CH₃)₃ | CH₂CH₃ |
| A-68 | (R)CH(CH₃)—C(CH₃)₃ | CH₂CH₂CH₃ |
| A-69 | (±)CH₂—CH(CH₃)—CH₂CH₃ | H |
| A-70 | (±)CH₂—CH(CH₃)—CH₂CH₃ | CH₃ |
| A-71 | (±)CH₂—CH(CH₃)—CH₂CH₃ | CH₂CH₃ |
| A-72 | (±)CH₂—CH(CH₃)—CH₂CH₃ | CH₂CH₂CH₃ |
| A-73 | (S)CH₂—CH(CH₃)—CH₂CH₃ | H |
| A-74 | (S)CH₂—CH(CH₃)—CH₂CH₃ | CH₃ |
| A-75 | (S)CH₂—CH(CH₃)—CH₂CH₃ | CH₂CH₃ |
| A-76 | (S)CH₂—CH(CH₃)—CH₂CH₃ | CH₂CH₂CH₃ |
| A-77 | (R)CH₂—CH(CH₃)—CH₂CH₃ | H |
| A-78 | (R)CH₂—CH(CH₃)—CH₂CH₃ | CH₃ |
| A-79 | (R)CH₂—CH(CH₃)—CH₂CH₃ | CH₂CH₃ |
| A-80 | (R)CH₂—CH(CH₃)—CH₂CH₃ | CH₂CH₂CH₃ |
| A-81 | (±)CH(CH₃)—CF₃ | H |
| A-82 | (±)CH(CH₃)—CF₃ | CH₃ |
| A-83 | (±)CH(CH₃)—CF₃ | CH₂CH₃ |
| A-84 | (±)CH(CH₃)—CF₃ | CH₂CH₂CH₃ |
| A-85 | (S)CH(CH₃)—CF₃ | H |
| A-86 | (S)CH(CH₃)—CF₃ | CH₃ |
| A-87 | (S)CH(CH₃)—CF₃ | CH₂CH₃ |
| A-88 | (S)CH(CH₃)—CF₃ | CH₂CH₂CH₃ |
| A-89 | (R)CH(CH₃)—CF₃ | H |
| A-90 | (R)CH(CH₃)—CF₃ | CH₃ |
| A-91 | (R)CH(CH₃)—CF₃ | CH₂CH₃ |
| A-92 | (R)CH(CH₃)—CF₃ | CH₂CH₂CH₃ |
| A-93 | (±)CH(CH₃)—CCl₃ | H |
| A-94 | (±)CH(CH₃)—CCl₃ | CH₃ |
| A-95 | (±)CH(CH₃)—CCl₃ | CH₂CH₃ |
| A-96 | (±)CH(CH₃)—CCl₃ | CH₂CH₂CH₃ |
| A-97 | (S)CH(CH₃)—CCl₃ | H |
| A-98 | (S)CH(CH₃)—CCl₃ | CH₃ |
| A-99 | (S)CH(CH₃)—CCl₃ | CH₂CH₃ |
| A-100 | (S)CH(CH₃)—CCl₃ | CH₂CH₂CH₃ |
| A-101 | (R)CH(CH₃)—CCl₃ | H |
| A-102 | (R)CH(CH₃)—CCl₃ | CH₃ |
| A-103 | (R)CH(CH₃)—CCl₃ | CH₂CH₃ |
| A-104 | (R)CH(CH₃)—CCl₃ | CH₂CH₂CH₃ |
| A-105 | CH₂CF₂CF₃ | H |
| A-106 | CH₂CF₂CF₃ | CH₃ |
| A-107 | CH₂CF₂CF₃ | CH₂CH₃ |
| A-108 | CH₂CF₂CF₃ | CH₂CH₂CH₃ |
| A-109 | CH₂(CF₂)₂CF₃ | H |
| A-110 | CH₂(CF₂)₂CF₃ | CH₃ |
| A-111 | CH₂(CF₂)₂CF₃ | CH₂CH₃ |
| A-112 | CH₂(CF₂)₂CF₃ | CH₂CH₂CH₃ |
| A-113 | CH₂C(CH₃)═CH₂ | H |
| A-114 | CH₂C(CH₃)═CH₂ | CH₃ |
| A-115 | CH₂C(CH₃)═CH₂ | CH₂CH₃ |
| A-116 | CH₂C(CH₃)═CH₂ | CH₂CH₂CH₃ |
| A-117 | CH₂CH═CH₂ | H |
| A-118 | CH₂CH═CH₂ | CH₃ |
| A-119 | CH₂CH═CH₂ | CH₂CH₃ |
| A-120 | CH(CH₃)CH═CH₂ | H |
| A-121 | CH(CH₃)CH═CH₂ | CH₃ |
| A-122 | CH(CH₃)CH═CH₂ | CH₂CH₃ |
| A-123 | CH(CH₃)CH═CH₂ | CH₂CH₂CH₃ |
| A-124 | CH(CH₃)C(CH₃)═CH₂ | H |
| A-125 | CH(CH₃)C(CH₃)═CH₂ | CH₃ |
| A-126 | CH(CH₃)C(CH₃)═CH₂ | CH₂CH₃ |
| A-127 | CH(CH₃)C(CH₃)═CH₂ | CH₂CH₂CH₃ |
| A-128 | CH₂—C≡CH | H |
| A-129 | CH₂—C≡CH | CH₃ |
| A-130 | CH₂—C≡CH | CH₂CH₃ |
| A-131 | CH₂—C≡CH | CH₂CH₂CH₃ |
| A-132 | Cyclopentyl | H |
| A-133 | Cyclopentyl | CH₃ |
| A-134 | Cyclopentyl | CH₂CH₃ |
| A-135 | Cyclopentyl | CH₂CH₂CH₃ |
| A-136 | Cyclohexyl | H |
| A-137 | Cyclohexyl | CH₃ |
| A-138 | Cyclohexyl | CH₂CH₃ |
| A-139 | Cyclohexyl | CH₂CH₂CH₃ |
| A-140 | CH₂—C₆H₅ | H |
| A-141 | CH₂—C₆H₅ | CH₃ |
| A-142 | CH₂—C₆H₅ | CH₂CH₃ |
| A-143 | CH₂—C₆H₅ | CH₂CH₂CH₃ |
| A-144 | NH₂ | CH₂-c-C₆H₁₁ |
| A-145 | NH₂ | CH₂CH₃ |
| A-146 | NH₂ | CH₂CH₂CH₃ |
| A-147 | NH—CH₂—CH═CH₂ | H |
| A-148 | NH—CH₂—CH═CH₂ | CH₃ |
| A-149 | NH—CH₂—CH═CH₂ | CH₂CH₃ |
| A-150 | NH—CH₂—CH═CH₂ | CH₂CH₂CH₃ |
| A-151 | NH—C(CH₃)₃ | H |
| A-152 | N(CH₃)₂ | H |
| A-153 | NH(CH₃) | H |
| A-154 | —(CH₂)₂CH═CHCH₂— | |
| A-155 | —(CH₂)₂C(CH₃)═CHCH₂— | |
| A-156 | —CH(CH₃)CH₂—CH═CHCH₂— | |
| A-157 | —(CH₂)₂CH(CH₃)(CH₂)₂— | |
| A-158 | —(CH₂)₃CHFCH₂— | |
| A-159 | —(CH₂)₂CHF(CH₂)₂— | |
| A-160 | —CH₂CHF(CH₂)₃— | |
| A-161 | —(CH₂)₂CH(CF₃)(CH₂)₂— | |
| A-162 | —(CH₂)₂O(CH₂)₂— | |
| A-163 | —(CH₂)₂S(CH₂)₂— | |
| A-164 | —(CH₂)₆— | |
| A-165 | —(CH₂)₅— | |
| A-166 | —(CH₂)₄— | |
| A-167 | —CH₂CH═CHCH₂— | |
| A-168 | —CH(CH₃)(CH₂)₃— | |
| A-169 | —CH₂CH(CH₃)(CH₂)₂— | |
| A-170 | —CH(CH₃)—(CH₂)₂—CH(CH₃)— | |
| A-171 | —CH(CH₃)—(CH₂)₄— | |
| A-172 | —CH₂—CH(CH₃)—(CH₂)₃— | |
| A-173 | —(CH₂)—CH(CH₃)—CH₂—CH(CH₃)—CH₂— | |
| A-174 | —CH(CH₂CH₃)—(CH₂)₄— | |
| A-175 | —(CH₂)₂—CHOH—(CH₂)₂— | |
| A-176 | —(CH₂)₆— | |
| A-177 | —CH(CH₃)—(CH₂)₅— | |
| A-178 | —(CH₂)₂—N(CH₃)—(CH₂)₂— | |
| A-179 | —N═CH—CH═CH— | |
| A-180 | —N═C(CH₃)—CH═C(CH₃)— | |
| A-181 | —N═C(CF₃)—CH═C(CF₃)— | |
| | H | CH₂CH₂OH |
| A-182 | CH₃ | CH₂CH₂OH |
| A-183 | CH₂CH₃ | CH₂CH₂OH |
| A-184 | CH₂CH₂CH₃ | CH₂CH₂OH |
| A-185 | H | CH(CH₃)CH₂OH |
| A-186 | CH₃ | CH(CH₃)CH₂OH |
| A-187 | CH₂CH₃ | CH(CH₃)CH₂OH |
| A-188 | CH₂CH₂CH₃ | CH(CH₃)CH₂OH |
| A-189 | H | CH(C₂H₅)CH₂OH |
| A-190 | CH₃ | CH(C₂H₅)CH₂OH |
| A-191 | CH₂CH₃ | CH(C₂H₅)CH₂OH |
| A-192 | CH₂CH₂CH₃ | CH(C₂H₅)CH₂OH |
| A-193 | H | CH(n-C₃H₇)CH₂OH |
| A-194 | CH₃ | CH(n-C₃H₇)CH₂OH |
| A-195 | CH₂CH₃ | CH(n-C₃H₇)CH₂OH |
| A-196 | CH₂CH₂CH₃ | CH(n-C₃H₇)CH₂OH |
| A-197 | H | CH(i-C₃H₇)CH₂OH |
| A-198 | CH₃ | CH(i-C₃H₇)CH₂OH |
| A-199 | CH₂CH₃ | CH(i-C₃H₇)CH₂OH |
| A-200 | CH₂CH₂CH₃ | CH(i-C₃H₇)CH₂OH |
| A-201 | H | CH(n-C₄H₉)CH₂OH |
| A-202 | CH₃ | CH(n-C₄H₉)CH₂OH |
| A-203 | CH₂CH₃ | CH(n-C₄H₉)CH₂OH |
| A-204 | CH₂CH₂CH₃ | CH(n-C₄H₉)CH₂OH |
| A-205 | H | CH(i-C₄H₉)CH₂OH |
| A-206 | CH₃ | CH(i-C₄H₉)CH₂OH |
| A-207 | CH₂CH₃ | CH(i-C₄H₉)CH₂OH |
| A-208 | CH₂CH₂CH₃ | CH(i-C₄H₉)CH₂OH |
| A-209 | H | CH₂CH₂OC(O)H |
| A-210 | CH₃ | CH₂CH₂OC(O)H |
| A-211 | CH₂CH₃ | CH₂CH₂OC(O)H |
| A-212 | CH₂CH₂CH₃ | CH₂CH₂OC(O)H |
| A-213 | H | CH(CH₃)CH₂OC(O)H |
| A-214 | CH₃ | CH(CH₃)CH₂OC(O)H |
| A-215 | CH₂CH₃ | CH(CH₃)CH₂OC(O)H |
| A-216 | CH₂CH₂CH₃ | CH(CH₃)CH₂OC(O)H |
| A-217 | H | CH(C₂H₅)CH₂OC(O)H |
| A-218 | CH₃ | CH(C₂H₅)CH₂OC(O)H |
| A-219 | CH₂CH₃ | CH(C₂H₅)CH₂OC(O)H |
| A-220 | CH₂CH₂CH₃ | CH(C₂H₅)CH₂OC(O)H |
| A-221 | H | CH(n-C₃H₇)CH₂OC(O)H |
| A-222 | CH₃ | CH(n-C₃H₇)CH₂OC(O)H |
| A-223 | CH₂CH₃ | CH(n-C₃H₇)CH₂OC(O)H |
| A-224 | CH₂CH₂CH₃ | CH(n-C₃H₇)CH₂OC(O)H |
| A-225 | H | CH(i-C₃H₇)CH₂OC(O)H |
| A-226 | CH₃ | CH(i-C₃H₇)CH₂OC(O)H |
| A-227 | CH₂CH₃ | CH(i-C₃H₇)CH₂OC(O)H |
| A-228 | CH₂CH₂CH₃ | CH(i-C₃H₇)CH₂OC(O)H |
| A-229 | H | CH(n-C₄H₉)CH₂OC(O)H |
| A-230 | CH₃ | CH(n-C₄H₉)CH₂OC(O)H |
| A-231 | CH₂CH₃ | CH(n-C₄H₉)CH₂OC(O)H |
| A-232 | CH₂CH₂CH₃ | CH(n-C₄H₉)CH₂OC(O)H |
| A-233 | H | CH(i-C₄H₉)CH₂OC(O)H |
| A-234 | CH₃ | CH(i-C₄H₉)CH₂OC(O)H |
| A-235 | CH₂CH₃ | CH(i-C₄H₉)CH₂OC(O)H |
| A-236 | CH₂CH₂CH₃ | CH(i-C₄H₉)CH₂OC(O)H |
| A-237 | H | CH₂CH₂OC(O)CH₃ |
| A-238 | CH₃ | CH₂CH₂OC(O)CH₃ |
| A-239 | CH₂CH₃ | CH₂CH₂OC(O)CH₃ |
| A-240 | CH₂CH₂CH₃ | CH₂CH₂OC(O)CH₃ |
| A-241 | H | CH(CH₃)CH₂OC(O)CH₃ |
| A-242 | CH₃ | CH(CH₃)CH₂OC(O)CH₃ |
| A-243 | CH₂CH₃ | CH(CH₃)CH₂OC(O)CH₃ |
| A-244 | CH₂CH₂CH₃ | CH(CH₃)CH₂OC(O)CH₃ |
| A-245 | H | CH(C₂H₅)CH₂OC(O)CH₃ |
| A-246 | CH₃ | CH(C₂H₅)CH₂OC(O)CH₃ |
| A-247 | CH₂CH₃ | CH(C₂H₅)CH₂OC(O)CH₃ |
| A-248 | CH₂CH₂CH₃ | CH(C₂H₅)CH₂OC(O)CH₃ |
| A-249 | H | CH(n-C₃H₇)CH₂OC(O)CH₃ |
| A-250 | CH₃ | CH(n-C₃H₇)CH₂OC(O)CH₃ |
| A-251 | CH₂CH₃ | CH(n-C₃H₇)CH₂OC(O)CH₃ |
| A-252 | CH₂CH₂CH₃ | CH(n-C₃H₇)CH₂OC(O)CH₃ |
| A-253 | H | CH(i-C₃H₇)CH₂OC(O)CH₃ |
| A-254 | CH₃ | CH(i-C₃H₇)CH₂OC(O)CH₃ |
| A-255 | CH₂CH₃ | CH(i-C₃H₇)CH₂OC(O)CH₃ |
| A-256 | CH₂CH₂CH₃ | CH(i-C₃H₇)CH₂OC(O)CH₃ |
| A-257 | H | CH(n-C₄H₉)CH₂OC(O)CH₃ |
| A-258 | CH₃ | CH(n-C₄H₉)CH₂OC(O)CH₃ |
| A-259 | CH₂CH₃ | CH(n-C₄H₉)CH₂OC(O)CH₃ |
| A-260 | CH₂CH₂CH₃ | CH(n-C₄H₉)CH₂OC(O)CH₃ |
| A-261 | H | CH(i-C₄H₉)CH₂OC(O)CH₃ |
| A-262 | CH₃ | CH(i-C₄H₉)CH₂OC(O)CH₃ |
| A-263 | CH₂CH₃ | CH(i-C₄H₉)CH₂OC(O)CH₃ |
| A-264 | CH₂CH₂CH₃ | CH(i-C₄H₉)CH₂OC(O)CH₃ |
| A-265 | H | CH₂CH₂OC(O)C₂H₅₃ |
| A-266 | CH₃ | CH₂CH₂OC(O)C₂H₅ |
| A-267 | CH₂CH₃ | CH₂CH₂OC(O)C₂H₅ |
| A-268 | CH₂CH₂CH₃ | CH₂CH₂OC(O)C₂H₅ |
| A-269 | H | CH(CH₃)CH₂OC(O)C₂H₅ |
| A-270 | CH₃ | CH(CH₃)CH₂OC(O)C₂H₅ |
| A-271 | CH₂CH₃ | CH(CH₃)CH₂OC(O)C₂H₅ |
| A-272 | CH₂CH₂CH₃ | CH(CH₃)CH₂OC(O)C₂H₅ |
| A-273 | H | CH(C₂H₅)CH₂OC(O)C₂H₅ |
| A-274 | CH₃ | CH(C₂H₅)CH₂OC(O)C₂H₅ |
| A-275 | CH₂CH₃ | CH(C₂H₅)CH₂OC(O)C₂H₅ |
| A-276 | CH₂CH₂CH₃ | CH(C₂H₅)CH₂OC(O)C₂H₅ |
| A-277 | H | CH(n-C₃H₇)CH₂OC(O)C₂H₅ |
| A-278 | CH₃ | CH(n-C₃H₇)CH₂OC(O)C₂H₅ |
| A-279 | CH₂CH₃ | CH(n-C₃H₇)CH₂OC(O)C₂H₅ |
| A-280 | CH₂CH₂CH₃ | CH(n-C₃H₇)CH₂OC(O)C₂H₅ |
| A-281 | H | CH(i-C₃H₇)CH₂OC(O)C₂H₅ |
| A-282 | CH₃ | CH(i-C₃H₇)CH₂OC(O)C₂H₅ |
| A-283 | CH₂CH₃ | CH(i-C₃H₇)CH₂OC(O)C₂H₅ |
| A-284 | CH₂CH₂CH₃ | CH(i-C₃H₇)CH₂OC(O)C₂H₅ |
| A-285 | H | CH(n-C₄H₉)CH₂OC(O)C₂H₅ |
| A-286 | CH₃ | CH(n-C₄H₉)CH₂OC(O)C₂H₅ |
| A-287 | CH₂CH₃ | CH(n-C₄H₉)CH₂OC(O)C₂H₅ |
| A-288 | CH₂CH₂CH₃ | CH(n-C₄H₉)CH₂OC(O)C₂H₅ |
| A-289 | H | CH(i-C₄H₉)CH₂OC(O)C₂H₅ |
| A-290 | CH₃ | CH(i-C₄H₉)CH₂OC(O)C₂H₅ |
| A-291 | CH₂CH₃ | CH(i-C₄H₉)CH₂OC(O)C₂H₅ |
| A-292 | CH₂CH₂CH₃ | CH(i-C₄H₉)CH₂OC(O)C₂H₅ |
| A-293 | H | CH₂CH₂OC(O)-n-C₃H₇ |
| A-294 | CH₃ | CH₂CH₂OC(O)-n-C₃H₇ |
| A-295 | CH₂CH₃ | CH₂CH₂OC(O)-n-C₃H₇ |
| A-296 | CH₂CH₂CH₃ | CH₂CH₂OC(O)-n-C₃H₇ |
| A-297 | H | CH(CH₃)CH₂OC(O)-n-C₃H₇ |
| A-298 | CH₃ | CH(CH₃)CH₂OC(O)-n-C₃H₇ |
| A-299 | CH₂CH₃ | CH(CH₃)CH₂OC(O)-n-C₃H₇ |
| A-300 | CH₂CH₂CH₃ | CH(CH₃)CH₂OC(O)-n-C₃H₇ |
| A-301 | H | CH(C₂H₅)CH₂OC(O)-n-C₃H₇ |
| A-302 | CH₃ | CH(C₂H₅)CH₂OC(O)-n-C₃H₇ |
| A-303 | CH₂CH₃ | CH(C₂H₅)CH₂OC(O)-n-C₃H₇ |
| A-304 | CH₂CH₂CH₃ | CH(C₂H₅)CH₂OC(O)-n-C₃H₇ |
| A-305 | H | CH(n-C₃H₇)CH₂OC(O)-n-C₃H₇ |
| A-306 | CH₃ | CH(n-C₃H₇)CH₂OC(O)-n-C₃H₇ |
| A-307 | CH₂CH₃ | CH(n-C₃H₇)CH₂OC(O)-n-C₃H₇ |
| A-308 | CH₂CH₂CH₃ | CH(n-C₃H₇)CH₂OC(O)-n-C₃H₇ |
| A-309 | H | CH(i-C₃H₇)CH₂OC(O)-n-C₃H₇ |
| A-310 | CH₃ | CH(i-C₃H₇)CH₂OC(O)-n-C₃H₇ |
| A-311 | CH₂CH₃ | CH(i-C₃H₇)CH₂OC(O)-n-C₃H₇ |
| A-312 | CH₂CH₂CH₃ | CH(i-C₃H₇)CH₂OC(O)-n-C₃H₇ |
| A-313 | H | CH(n-C₄H₉)CH₂OC(O)-n-C₃H₇ |
| A-314 | CH₃ | CH(n-C₄H₉)CH₂OC(O)-n-C₃H₇ |
| A-315 | CH₂CH₃ | CH(n-C₄H₉)CH₂OC(O)-n-C₃H₇ |
| A-316 | CH₂CH₂CH₃ | CH(n-C₄H₉)CH₂OC(O)-n-C₃H₇ |
| A-317 | H | CH(i-C₄H₉)CH₂OC(O)-n-C₃H₇ |
| A-318 | CH₃ | CH(i-C₄H₉)CH₂OC(O)-n-C₃H₇ |
| A-319 | CH₂CH₃ | CH(i-C₄H₉)CH₂OC(O)-n-C₃H₇ |
| A-320 | CH₂CH₂CH₃ | CH(i-C₄H₉)CH₂OC(O)-n-C₃H₇ |
| A-321 | H | CH₂CH₂OC(O)CH(CH₂)₂ |
| A-322 | CH₃ | CH₂CH₂OC(O)CH(CH₂)₂ |
| A-323 | CH₂CH₃ | CH₂CH₂OC(O)CH(CH₂)₂ |
| A-324 | CH₂CH₂CH₃ | CH₂CH₂OC(O)CH(CH₂)₂ |
| A-325 | H | CH(CH₃)CH₂OC(O)CH(CH₂)₂ |
| A-326 | CH₃ | CH(CH₃)CH₂OC(O)CH(CH₂)₂ |
| A-327 | CH₂CH₃ | CH(CH₃)CH₂OC(O)CH(CH₂)₂ |
| A-328 | CH₂CH₂CH₃ | CH(CH₃)CH₂OC(O)CH(CH₂)₂ |
| A-329 | H | CH(C₂H₅)CH₂OC(O)CH(CH₂)₂ |
| A-330 | CH₃ | CH(C₂H₅)CH₂OC(O)CH(CH₂)₂ |
| A-331 | CH₂CH₃ | CH(C₂H₅)CH₂OC(O)CH(CH₂)₂ |
| A-332 | CH₂CH₂CH₃ | CH(C₂H₅)CH₂OC(O)CH(CH₂)₂ |
| A-333 | H | CH(n-C₃H₇)CH₂OC(O)CH(CH₂)₂ |
| A-334 | CH₃ | CH(n-C₃H₇)CH₂OC(O)CH(CH₂)₂ |
| A-335 | CH₂CH₃ | CH(n-C₃H₇)CH₂OC(O)CH(CH₂)₂ |
| A-336 | CH₂CH₂CH₃ | CH(n-C₃H₇)CH₂OC(O)CH(CH₂)₂ |
| A-337 | H | CH(i-C₃H₇)CH₂OC(O)CH(CH₂)₂ |
| A-338 | CH₃ | CH(i-C₃H₇)CH₂OC(O)CH(CH₂)₂ |
| A-339 | CH₂CH₃ | CH(i-C₃H₇)CH₂OC(O)CH(CH₂)₂ |
| A-340 | CH₂CH₂CH₃ | CH(i-C₃H₇)CH₂OC(O)CH(CH₂)₂ |
| A-341 | H | CH(n-C₄H₉)—CH₂OC(O)CH(CH₂)₂ |
| A-342 | CH₃ | CH(n-C₄H₉)CH₂OC(O)CH(CH₂)₂ |
| A-343 | CH₂CH₃ | CH(n-C₄H₉)CH₂OC(O)CH(CH₂)₂ |
| A-344 | CH₂CH₂CH₃ | CH(n-C₄H₉)CH₂OC(O)CH(CH₂)₂ |
| A-345 | H | CH(i-C₄H₉)CH₂OC(O)CH(CH₂)₂ |
| A-346 | CH₃ | CH(i-C₄H₉)CH₂OC(O)CH(CH₂)₂ |
| A-347 | CH₂CH₃ | CH(i-C₄H₉)CH₂OC(O)CH(CH₂)₂ |
| A-348 | CH₂CH₂CH₃ | CH(i-C₄H₉)CH₂OC(O)CH(CH₂)₂ |

TABLE B

| No. | R^{1′} |
|---|---|
| B-1 | CH₃ |
| B-2 | CH₂CH₃ |
| B-3 | CH₂CH₂CH₃ |
| B-4 | CH(CH₃)₂ |
| B-5 | CH₂CH(CH₃)₂ |
| B-6 | (±)CH(CH₃)CH₂CH₃ |
| B-7 | (R)CH(CH₃)CH₂CH₃ |
| B-8 | (S)CH(CH₃)CH₂CH₃ |
| B-9 | (CH₂)₃CH₃ |
| B-10 | C(CH₃)₃ |
| B-11 | (CH₂)₄CH₃ |
| B-12 | CH(CH₂CH₃)₂ |
| B-13 | CH₂CH₂CH(CH₃)₂ |
| B-14 | (±)CH(CH₃)(CH₂)₂CH₃ |
| B-15 | (R)CH(CH₃)(CH₂)₂CH₃ |
| B-16 | (S)CH(CH₃)(CH₂)₂CH₃ |
| B-17 | (±)CH₂CH(CH₃)CH₂CH₃ |
| B-18 | (R)CH₂CH(CH₃)CH₂CH₃ |
| B-19 | (S)CH₂CH(CH₃)CH₂CH₃ |
| B-20 | (±)CH(CH₃)CH(CH₃)₂ |
| B-21 | (R)CH(CH₃)CH(CH₃)₂ |
| B-22 | (S)CH(CH₃)CH(CH₃)₂ |
| B-23 | (CH₂)₅CH₃ |
| B-24 | (±,±)CH(CH₃)CH(CH₃)CH₂CH₃ |
| B-25 | (±,R)CH(CH₃)CH(CH₃)CH₂CH₃ |
| B-26 | (±,S)CH(CH₃)CH(CH₃)CH₂CH₃ |
| B-27 | (R,±)CH(CH₃)CH(CH₃)CH₂CH₃ |
| B-28 | (S,±)CH(CH₃)CH(CH₃)CH₂CH₃ |
| B-29 | (±)CH₂CH(CH₃)CF₃ |
| B-30 | (R)CH₂CH(CH₃)CF₃ |
| B-31 | (S)CH₂CH(CH₃)CF₃ |
| B-32 | (±)CH₂CH(CF₃)CH₂CH₃ |
| B-33 | (R)CH₂CH(CF₃)CH₂CH₃ |
| B-34 | (S)CH₂CH(CF₃)CH₂CH₃ |
| B-35 | (±,±)CH(CH₃)CH(CH₃)CF₃ |
| B-36 | (±,R)CH(CH₃)CH(CH₃)CF₃ |
| B-37 | (±,S)CH(CH₃)CH(CH₃)CF₃ |
| B-38 | (R,±)CH(CH₃)CH(CH₃)CF₃ |
| B-39 | (S,±)CH(CH₃)CH(CH₃)CF₃ |
| B-40 | (±,±)CH(CH₃)CH(CF₃)CH₂CH₃ |
| B-41 | (±,R)CH(CH₃)CH(CF₃)CH₂CH₃ |
| B-42 | (±,S)CH(CH₃)CH(CF₃)CH₂CH₃ |
| B-43 | (R,±)CH(CH₃)CH(CF₃)CH₂CH₃ |
| B-44 | (S,±)CH(CH₃)CH(CF₃)CH₂CH₃ |
| B-45 | CF₃ |
| B-46 | CF₂CF₃ |
| B-47 | CF₂CF₂CF₃ |
| B-48 | c-C₃H₅ |
| B-49 | (1-CH₃)-c-C₃H₄ |
| B-50 | c-C₅H₉ |
| B-51 | c-C₆H₁₁ |
| B-52 | (4-CH₃)-c-C₆H₁₀ |
| B-53 | CH₂C(CH₃)═CH₂ |
| B-54 | CH₂CH₂C(CH₃)═CH₂ |
| B-55 | CH₂—C(CH₃)₃ |
| B-56 | CH₂—Si(CH₃)₃ |
| B-57 | n-C₆H₁₃ |
| B-58 | (CH₂)₃—CH(CH₃)₂ |
| B-59 | (CH₂)₂—CH(CH₃)—C₂H₅ |
| B-60 | CH₂—CH(CH₃)-n-C₃H₇ |
| B-61 | CH(CH₃)-n-C₄H₉ |
| B-62 | CH₂—CH(C₂H₅)₂ |
| B-63 | CH(C₂H₅)-n-C₃H₇ |
| B-64 | CH₂-c-C₅H₉ |
| B-65 | CH₂—CH(CH₃)—CH(CH₃)₂ |
| B-66 | CH(CH₃)—CH₂CH(CH₃)₂ |
| B-67 | CH(CH₃)—CH(CH₃)—C₂H₅ |
| B-68 | CH(CH₃)—C(CH₃)₃ |
| B-69 | (CH₂)₂—C(CH₃)₃ |
| B-70 | CH₂—C(CH₃)₂—C₂H₅ |
| B-71 | 2-CH₃-c-C₅H₈ |
| B-72 | 3-CH₃-c-C₅H₈ |
| B-73 | C(CH₃)₂-n-C₃H₇ |
| B-74 | (CH₂)₆—CH₃ |
| B-75 | (CH₂)₄—CH(CH₃)₂ |
| B-76 | (CH₂)₃—CH(CH₃)—C₂H₅ |
| B-77 | (CH₂)₂—CH(CH₃)-n-C₃H₇ |
| B-78 | CH₂—CH(CH₃)-n-C₄H₉ |
| B-79 | CH(CH₃)-n-C₅H₁₁ |
| B-80 | (CH₂)₃C(CH₃)₃ |
| B-81 | (CH₂)₂CH(CH₃)—CH(CH₃)₂ |
| B-82 | (CH₂)CH(CH₃)—CH₂CH(CH₃)₂ |
| B-83 | CH(CH₃)(CH₂)₂—CH(CH₃)₂ |
| B-84 | (CH₂)₂C(CH₃)₂C₂H₅ |
| B-85 | CH₂CH(CH₃)CH(CH₃)C₂H₅ |
| B-86 | CH(CH₃)CH₂CH(CH₃)C₂H₅ |
| B-87 | CH₂C(CH₃)₂-n-C₃H₇ |
| B-88 | CH(CH₃)CH(CH₃)-n-C₃H₇ |
| B-89 | C(CH₃)₂-n-C₄H₉ |
| B-90 | (CH₂)₂CH(C₂H₅)₂ |
| B-91 | CH₂CH(C₂H₅)-n-C₃H₇ |
| B-92 | CH(C₂H₅)-n-C₄H₉ |
| B-93 | CH₂CH(CH₃)C(CH₃)₃ |
| B-94 | CH(CH₃)CH₂C(CH₃)₃ |
| B-95 | CH₂C(CH₃)₂CH(CH₃)₂ |
| B-96 | CH₂CH(C₂H₅)CH(CH₃)₂ |
| B-97 | CH(CH₃)CH(CH₃)CH(CH₃)₂ |
| B-98 | C(CH₃)₂CH₂CH(CH₃)₂ |
| B-99 | CH(C₂H₅)CH₂CH(CH₃)₂ |
| B-100 | CH(CH₃)C(CH₃)₂C₂H₅ |
| B-101 | CH(CH₃)CH(C₂H₅)₂ |
| B-102 | C(CH₃)₂CH(CH₃)C₂H₅ |
| B-103 | CH(C₂H₅)CH(CH₃)C₂H₅ |
| B-104 | C(CH₃)(C₂H₅)-n-C₃H₇ |
| B-105 | CH(n-C₃H₇)₂ |
| B-106 | CH(n-C₃H₇)CH(CH₃)₂ |
| B-107 | C(CH₃)₂C(CH₃)₃ |
| B-108 | C(CH₃)(C₂H₅)—CH(CH₃)₂ |
| B-109 | C(C₂H₅)₃ |
| B-110 | (3-CH₃)-c-C₆H₁₀ |
| B-111 | (2-CH₃)-c-C₆H₁₀ |
| B-112 | n-C₈H₁₇ |
| B-113 | CH₂C(═NO—CH₃)CH₃ |
| B-114 | CH₂C(═NO—C₂H₅)CH₃ |
| B-115 | CH₂C(═NO-n-C₃H₇)CH₃ |
| B-116 | CH₂C(═NO-i-C₃H₇)CH₃ |
| B-117 | CH(CH₃)C(═NOCH₃)CH₃ |
| B-118 | CH(CH₃)C(═NOC₂H₅)CH₃ |
| B-119 | CH(CH₃)C(═NO-n-C₃H₇)CH₃ |
| B-120 | CH(CH₃)C(═NO-i-C₃H₇)CH₃ |
| B-121 | C(═NOCH₃)C(═NOCH₃)CH₃ |
| B-122 | C(═NOCH₃)C(═NOC₂H₅)CH₃ |
| B-123 | C(═NOCH₃)C(═NO-n-C₃H₇)CH₃ |
| B-124 | C(═NOCH₃)C(═NO-i-C₃H₇)CH₃ |
| B-125 | C(═NOC₂H₅)C(═NOCH₃)CH₃ |
| B-126 | C(═NOC₂H₅)C(═NOC₂H₅)CH₃ |
| B-127 | C(═NOC₂H₅)C(═NO-n-C₃H₇)CH₃ |
| B-128 | C(═NOC₂H₅)C(═NO-i-C₃H₇)CH₃ |
| B-129 | CH₂C(═NO—CH₃)C₂H₅ |
| B-130 | CH₂C(═NO—C₂H₅)C₂H₅ |
| B-131 | CH₂C(═NO-n-C₃H₇)C₂H₅ |
| B-132 | CH₂C(═NO-i-C₃H₇)C₂H₅ |
| B-133 | CH(CH₃)C(═NOCH₃)C₂H₅ |
| B-134 | CH(CH₃)C(═NOC₂H₅)C₂H₅ |
| B-135 | CH(CH₃)C(═NO-n-C₃H₇)C₂H₅ |
| B-136 | CH(CH₃)C(═NO-i-C₃H₇)C₂H₅ |
| B-137 | C(═NOCH₃)C(═NOCH₃)C₂H₅ |
| B-138 | C(═NOCH₃)C(═NOC₂H₅)C₂H₅ |
| B-139 | C(═NOCH₃)C(═NO-n-C₃H₇)C₂H₅ |
| B-140 | C(═NOCH₃)C(═NO-i-C₃H₇)C₂H₅ |
| B-141 | C(═NOC₂H₅)C(═NOCH₃)C₂H₅ |
| B-142 | C(═NOC₂H₅)C(═NOC₂H₅)C₂H₅ |
| B-143 | C(═NOC₂H₅)C(═NO-n-C₃H₇)C₂H₅ |
| B-144 | C(═NOC₂H₅)C(═NO-i-C₃H₇)C₂H₅ |
| B-145 | CH═CH—CH₂CH₃ |
| B-146 | CH₂—CH═CH—CH₃ |
| B-147 | CH₂—CH₂—CH═CH₂ |
| B-148 | C(CH₃)₂CH₂CH₃ |
| B-149 | CH═C(CH₃)₂ |
| B-150 | C(═CH₂)—CH₂CH₃ |
| B-151 | C(CH₃)═CH—CH₃ |
| B-152 | CH(CH₃)CH═CH₂ |
| B-153 | CH═CH-n-C₃H₇ |
| B-154 | CH₂—CH═CH—C₂H₅ |
| B-155 | (CH₂)₂—CH═CH—CH₃ |
| B-156 | (CH₂)₃—CH═CH₂ |
| B-157 | CH═CH—CH(CH₃)₂ |
| B-158 | CH₂—CH═C(CH₃)₂ |
| B-159 | (CH₂)₂—C(CH₃)═CH₂ |
| B-160 | CH═C(CH₃)—C₂H₅ |
| B-161 | CH₂—C(═CH₂)—C₂H₅ |
| B-162 | CH₂—C(CH₃)═CH—CH₃ |
| B-163 | CH₂—CH(CH₃)—CH═CH₂ |
| B-164 | C(═CH₂)—CH₂—CH₂—CH₃ |
| B-165 | C(CH₃)═CH—CH₂—CH₃ |
| B-166 | CH(CH₃)—CH═CH—CH₃ |
| B-167 | CH(CH₃)—CH₂—CH═CH₂ |
| B-168 | C(═CH₂)CH(CH₃)₂ |
| B-169 | C(CH₃)═C(CH₃)₂ |
| B-170 | CH(CH₃)—C(═CH₂)—CH₃ |
| B-171 | C(CH₃)₂—CH═CH₂ |
| B-172 | C(C₂H₅)═CH—CH₃ |
| B-173 | CH(C₂H₅)—CH═CH₂ |
| B-174 | CH═CH—CH₂—CH₂—CH₂—CH₃ |
| B-175 | CH₂—CH═CH—CH₂—CH₂—CH₃ |
| B-176 | CH₂—CH₂—CH═CH—CH₂—CH₃ |
| B-177 | CH₂—CH₂—CH₂—CH═CH—CH₃ |
| B-178 | CH₂—CH₂—CH₂—CH₂—CH═CH₂ |
| B-179 | CH═CH—CH₂—CH(CH₃)CH₃ |
| B-180 | CH₂—CH═CH—CH(CH₃)CH₃ |
| B-181 | CH₂—CH₂—CH═C(CH₃)CH₃ |
| B-182 | CH₂—CH₂—CH₂—C(CH₃)═CH₂ |
| B-183 | CH═CH—CH(CH₃)—CH₂—CH₃ |
| B-184 | CH₂—CH═C(CH₃)—CH₂—CH₃ |
| B-185 | CH₂—CH₂—C(═CH₂)—CH₂—CH₃ |
| B-186 | CH₂—CH₂—C(CH₃)═CH—CH₃ |
| B-187 | CH₂—CH₂—CH(CH₃)—CH═CH₂ |
| B-188 | CH═C(CH₃)—CH₂—CH₂—CH₃ |
| B-189 | CH₂—C(═CH₂)—CH₂—CH₂—CH₃ |
| B-190 | CH₂—C(CH₃)═CH—CH₂—CH₃ |
| B-191 | CH₂—CH(CH₃)—CH═CH—CH₃ |
| B-192 | CH₂—CH(CH₃)—CH₂—CH═CH₂ |
| B-193 | C(═CH₂)—CH₂—CH₂—CH₂—CH₃ |
| B-194 | C(CH₃)═CH—CH₂—CH₂—CH₃ |
| B-195 | CH(CH₃)—CH═CH—CH₂—CH₃ |
| B-196 | CH(CH₃)—CH₂—CH═CH—CH₃ |
| B-197 | CH(CH₃)—CH₂—CH₂—CH═CH₂ |
| B-198 | CH═CH—C(CH₃)₃ |
| B-199 | CH═C(CH₃)—CH(CH₃)—CH₃ |
| B-200 | CH₂—C(═CH₂)—CH(CH₃)—CH₃ |
| B-201 | CH₂—C(CH₃)═C(CH₃)—CH₃ |
| B-202 | CH₂—CH(CH₃)—C(═CH₂)—CH₃ |
| B-203 | C(═CH₂)—CH₂—CH(CH₃)—CH₃ |
| B-204 | C(CH₃)═CH—CH(CH₃)—CH₃ |
| B-205 | CH(CH₃)—CH═C(CH₃)—CH₃ |
| B-206 | CH(CH₃)—CH₂—C(═CH₂)—CH₃ |
| B-207 | CH═C(CH₂—CH₃)—CH₂—CH₃ |
| B-208 | CH₂—C(═CH—CH₃)—CH₂—CH₃ |
| B-209 | CH₂—CH(CH═CH₂)—CH₂—CH₃ |
| B-210 | C(═CH—CH₃)—CH₂—CH₂—CH₃ |
| B-211 | CH(CH═CH₂)—CH₂—CH₂—CH₃ |
| B-212 | C(CH₂—CH₃)═CH—CH₂—CH₃ |
| B-213 | CH(CH₂—CH₃)—CH═CH—CH₃ |
| B-214 | CH(CH₂—CH₃)—CH₂—CH═CH₂ |
| B-215 | CH₂—C(CH₃)₂—CH═CH₂ |
| B-216 | C(═CH₂)—CH(CH₃)—CH₂—CH₃ |
| B-217 | C(CH₃)═C(CH₃)—CH₂—CH₃ |
| B-218 | CH(CH₃)—C(═CH₂)—CH₂—CH₃ |
| B-219 | CH(CH₃)—C(CH₃)═CH—CH₃ |
| B-220 | CH(CH₃)—CH(CH₃)—CH═CH₂ |
| B-221 | C(CH₃)₂—CH═CH—CH₃ |
| B-222 | C(CH₃)₂—CH₂—CH═CH₂ |
| B-223 | C(═CH₂)—C(CH₃)₃ |
| B-224 | C(═CH—CH₃)—CH(CH₃)—CH₃ |
| B-225 | CH(CH═CH₂)—CH(CH₃)—CH₃ |
| B-226 | C(CH₂—CH₃)═C(CH₃)—CH₃ |
| B-227 | CH(CH₂—CH₃)—C(═CH₂)—CH₃ |
| B-228 | C(CH₃)₂—C(═CH₂)—CH₃ |
| B-229 | C(CH₃)(CH═CH₂)—CH₂—CH₃ |
| B-230 | C(CH₃)(CH₂CH₃)—CH₂—CH₂—CH₃ |
| B-231 | CH(CH₂CH₃)—CH(CH₃)—CH₂—CH₃ |
| B-232 | CH(CH₂CH₃)—CH₂—CH(CH₃)—CH₃ |
| B-233 | C(CH₃)₂—C(CH₃)₃ |
| B-234 | C(CH₂—CH₃)—C(CH₃)₃ |
| B-235 | C(CH₃)(CH₂—CH₃)—CH(CH₃)₂ |
| B-236 | CH(CH(CH₃)₂)—CH(CH₃)₂ |
| B-237 | CH═CH—CH₂—CH₂—CH₂—CH₂—CH₃ |
| B-238 | CH₂—CH═CH—CH₂—CH₂—CH₂—CH₃ |
| B-239 | CH₂—CH₂—CH═CH—CH₂—CH₂—CH₃ |
| B-240 | CH₂—CH₂—CH₂—CH═CH—CH₂—CH₃ |
| B-241 | CH₂—CH₂—CH₂—CH₂—CH═CH—CH₃ |
| B-242 | CH₂—CH₂—CH₂—CH₂—CH₂—CH═CH₂ |
| B-243 | CH═CH—CH₂—CH₂—CH(CH₃)—CH₃ |
| B-244 | CH₂—CH═CH—CH₂—CH(CH₃)—CH₃ |
| B-245 | CH₂—CH₂—CH═CH—CH(CH₃)—CH₃ |
| B-246 | CH₂—CH₂—CH₂—CH═C(CH₃)—CH₃ |
| B-247 | CH₂—CH₂—CH₂—CH₂—C(═CH₂)—CH₃ |
| B-248 | CH═CH—CH₂—CH(CH₃)—CH₂—CH₃ |
| B-249 | CH₂—CH═CH—CH(CH₃)—CH₂—CH₃ |
| B-250 | CH₂—CH₂—CH═C(CH₃)—CH₂—CH₃ |
| B-251 | CH₂—CH₂—CH₂—C(═CH₂)—CH₂—CH₃ |
| B-252 | CH₂—CH₂—CH₂—C(CH₃)═CH—CH₃ |
| B-253 | CH₂—CH₂—CH₂—CH(CH₃)—CH═CH₂ |
| B-254 | CH═CH—CH(CH₃)—CH₂—CH₂—CH₃ |
| B-255 | CH₂—CH═C(CH₃)—CH₂—CH₂—CH₃ |
| B-256 | CH₂—CH₂—C(═CH₂)—CH₂—CH₂—CH₃ |
| B-257 | CH₂—CH₂—C(CH₃)═CH—CH₂—CH₃ |
| B-258 | CH₂—CH₂—CH(CH₃)—CH═CH—CH₃ |
| B-259 | CH₂—CH₂—CH(CH₃)—CH₂—CH═CH₂ |
| B-260 | CH═C(CH₃)—CH₂—CH₂—CH₂—CH₃ |
| B-261 | CH₂—C(═CH₂)—CH₂—CH₂—CH₂—CH₃ |
| B-262 | CH₂—C(CH₃)═CH—CH₂—CH₂—CH₃ |
| B-263 | CH₂—CH(CH₃)—CH═CH—CH₂—CH₃ |
| B-264 | CH₂—CH(CH₃)—CH₂—CH═CH—CH₃ |
| B-265 | CH₂—CH(CH₃)—CH₂—CH₂—CH═CH₂ |
| B-266 | C(═CH₂)—CH₂—CH₂—CH₂—CH₂—CH₃ |
| B-267 | C(CH₃)═CH—CH₂—CH₂—CH₂—CH₃ |
| B-268 | CH(CH₃)—CH═CH—CH₂—CH₂—CH₃ |
| B-269 | CH(CH₃)—CH₂—CH═CH—CH₂—CH₃ |
| B-270 | CH(CH₃)—CH₂—CH₂—CH═CH—CH₃ |
| B-271 | CH(CH₃)—CH₂—CH₂—CH₂—CH═CH₂ |
| B-272 | CH═CH—CH₂—C(CH₃)₃ |
| B-273 | CH₂—CH═CH—C(CH₃)₃ |
| B-274 | CH═CH—CH(CH₃)—CH(CH₃)₂ |
| B-275 | CH₂—CH═C(CH₃)—CH(CH₃)₂ |
| B-276 | CH₂—CH₂—C(═CH₂)—CH(CH₃)₂ |
| B-277 | CH₂—CH₂—C(CH₃)═C(CH₃)₂ |
| B-278 | CH₂—CH₂—CH(CH₃)—C(═CH₂)—CH₃ |
| B-279 | CH═C(CH₃)—CH₂—CH(CH₃)₂ |
| B-280 | CH₂—C(═CH₂)—CH₂—CH(CH₃)₂ |
| B-281 | CH₂—C(CH₃)═CH—CH(CH₃)₂ |
| B-282 | CH₂—CH(CH₃)—CH═C(CH₃)₂ |
| B-283 | CH₂—CH(CH₃)—CH₂—C(═CH₂)—CH₃ |
| B-284 | C(═CH₂)—CH₂—CH₂—CH(CH₃)₂ |
| B-285 | C(CH₃)═CH—CH₂—CH(CH₃)₂ |
| B-286 | CH(CH₃)—CH═CH—CH(CH₃)₂ |
| B-287 | CH(CH₃)—CH₂—CH═C(CH₃)₂ |
| B-288 | CH(CH₃)—CH₂—CH₂—C(═CH₂)—CH₃ |
| B-289 | CH═CH—C(CH₃)₂—CH₂—CH₃ |
| B-290 | CH₂—CH₂—C(CH₃)₂—CH═CH₂ |
| B-291 | CH═C(CH₃)—CH(CH₃)—CH₂—CH₃ |
| B-292 | CH₂—C(═CH₂)—CH(CH₃)—CH₂—CH₃ |
| B-293 | CH₂—C(CH₃)═C(CH₃)—CH₂—CH₃ |
| B-294 | CH₂—CH(CH₃)—C(═CH₂)—CH₂—CH₃ |
| B-295 | CH₂—CH(CH₃)—C(CH₃)═CH—CH₃ |
| B-296 | CH₂—CH(CH₃)—CH(CH₃)—CH═CH₂ |
| B-297 | C(═CH₂)—CH₂—CH(CH₃)—CH₂—CH₃ |
| B-298 | C(CH₃)═CH—CH(CH₃)—CH₂—CH₃ |
| B-299 | CH(CH₃)—CH═C(CH₃)—CH₂—CH₃ |
| B-300 | CH(CH₃)—CH₂—C(═CH₂)—CH₂—CH₃ |
| B-301 | CH(CH₃)—CH₂—C(CH₃)═CH—CH₃ |
| B-302 | CH(CH₃)—CH₂—CH(CH₃)—CH═CH₂ |
| B-303 | CH₂—C(CH₃)₂—CH═CH—CH₃ |
| B-304 | CH₂—C(CH₃)₂—CH₂—CH═CH₂ |
| B-305 | C(═CH₂)—CH(CH₃)—CH₂—CH₂—CH₃ |
| B-306 | C(CH₃)═C(CH₃)—CH₂—CH₂—CH₃ |
| B-307 | CH(CH₃)—C(═CH₂)—CH₂—CH₂—CH₃ |
| B-308 | CH(CH₃)—C(CH₃)═CH—CH₂—CH₃ |
| B-309 | CH(CH₃)—CH(CH₃)—CH═CH—CH₃ |
| B-310 | CH(CH₃)—CH(CH₃)—CH₂—CH═CH₂ |
| B-311 | C(CH₃)₂—CH═CH—CH₂—CH₃ |
| B-312 | C(CH₃)₂—CH₂—CH═CH—CH₃ |
| B-313 | C(CH₃)₂—CH₂—CH₂—CH═CH₂ |
| B-314 | CH═CH—CH(CH₂—CH₃)—CH₂—CH₃ |
| B-315 | CH₂—CH═C(CH₂—CH₃)—CH₂—CH₃ |
| B-316 | CH₂—CH₂—C(═CH—CH₃)—CH₂—CH₃ |
| B-317 | CH₂—CH₂—CH(CH═CH₂)—CH₂—CH₃ |
| B-318 | CH═C(CH₂—CH₃)—CH₂—CH₂—CH₃ |
| B-319 | CH₂—C(═CH—CH₃)—CH₂—CH₂—CH₃ |
| B-320 | CH₂—CH(CH═CH₂)—CH₂—CH₂—CH₃ |
| B-321 | CH₂—C(CH₂—CH₃)═CH—CH₂—CH₃ |
| B-322 | CH₂—CH(CH₂—CH₃)—CH═CH—CH₃ |
| B-323 | CH₂—CH(CH₂—CH₃)—CH—CH═CH₂ |
| B-324 | C(═CH—CH₃)—CH₂—CH₂—CH₂—CH₃ |
| B-325 | CH(CH═CH₂)—CH₂—CH₂—CH₂—CH₃ |
| B-326 | C(CH₂—CH₃)═CH—CH₂—CH₂—CH₃ |
| B-327 | CH(CH₂—CH₃)—CH═CH—CH₂—CH₃ |
| B-328 | CH(CH₂—CH₃)—CH₂—CH═CH—CH₃ |
| B-329 | CH(CH₂—CH₃)—CH₂—CH₂—CH═CH₂ |
| B-330 | C(═CH—CH₂—CH₃)—CH₂—CH₂—CH₃ |
| B-331 | C(CH═CH—CH₃)—CH₂—CH₂—CH₃ |
| B-332 | C(CH₂—CH═CH₂)—CH₂—CH₂—CH₃ |
| B-333 | CH═C(CH₃)—C(CH₃)₃ |
| B-334 | CH₂—C(═CH₂)—C(CH₃)₃ |
| B-335 | CH₂—C(CH₃)₂—CH(═CH₂)—CH₃ |
| B-336 | C(═CH₂)—CH(CH₃)—CH(CH₃)—CH₃ |
| B-337 | C(CH₃)═C(CH₃)—CH(CH₃)—CH₃ |
| B-338 | CH(CH₃)—C(═CH₂)—CH(CH₃)—CH₃ |
| B-339 | CH(CH₃)—C(CH₃)═C(CH₃)—CH₃ |
| B-340 | CH(CH₃)—CH(CH₃)—C(═CH₂)—CH₃ |
| B-341 | C(CH₃)₂—CH═C(CH₃)—CH₃ |
| B-342 | C(CH₃)₂—CH₂—C(═CH₂)—CH₃ |
| B-343 | C(CH₃)₂—C(═CH₂)—CH₂—CH₃ |
| B-344 | C(CH₃)₂—C(CH₃)═CH—CH₃ |
| B-345 | C(CH₃)₂—CH(CH₃)CH═CH₂ |
| B-346 | CH(CH₂—CH₃)—CH₂—CH(CH₃)—CH₃ |
| B-347 | CH(CH₂—CH₃)—CH(CH₃)—CH₂—CH₃ |
| B-348 | C(CH₃)(CH₂—CH₃)—CH₂—CH₂—CH₃ |
| B-349 | CH(i-C₃H₇)—CH₂—CH₂—CH₃ |
| B-350 | CH═C(CH₂—CH₃)—CH(CH₃)—CH₃ |
| B-351 | CH₂—C(═CH—CH₃)—CH(CH₃)—CH₃ |
| B-352 | CH₂—CH(CH═CH₂)—CH(CH₃)—CH₃ |
| B-353 | CH₂—C(CH₂—CH₃)═C(CH₃)—CH₃ |
| B-354 | CH₂—CH(CH₂—CH₃)—C(═CH₂)—CH₃ |
| B-355 | CH₂—C(CH₃)(CH═CH₂)—CH₂—CH₃ |
| B-356 | C(═CH₂)—CH(CH₂—CH₃)—CH₂—CH₃ |
| B-357 | C(CH₃)═C(CH₂—CH₃)—CH₂—CH₃ |
| B-358 | CH(CH₃)—C(═CH—CH₃)—CH₂—CH₃ |
| B-359 | CH(CH₃)—CH(CH═CH₂)—CH₂—CH₃ |
| B-360 | CH═C(CH₂—CH₃)—CH(CH₃)—CH₃ |
| B-361 | CH₂—C(═CH—CH₃)—CH(CH₃)—CH₃ |
| B-362 | CH₂—CH(CH═CH₂)—CH(CH₃)—CH₃ |
| B-363 | CH₂—C(CH₂—CH₃)═C(CH₃)—CH₃ |
| B-364 | CH₂—CH(CH₂—CH₃)—C(═CH₂)—CH₃ |
| B-365 | C(═CH—CH₃)—CH₂—CH(CH₃)—CH₃ |
| B-366 | CH(CH═CH₂)—CH₂—CH(CH₃)—CH₃ |
| B-367 | C(CH₂—CH₃)═CH—CH(CH₃)—CH₃ |
| B-368 | CH(CH₂—CH₃)CH═C(CH₃)—CH₃ |
| B-369 | CH(CH₂—CH₃)CH₂—C(═CH₂)—CH₃ |
| B-370 | C(═CH—CH₃)CH(CH₃)—CH₂—CH₃ |
| B-371 | CH(CH═CH₂)CH(CH₃)—CH₂—CH₃ |
| B-372 | C(CH₂—CH₃)═C(CH₃)—CH₂—CH₃ |
| B-373 | CH(CH₂—CH₃)—C(═CH₂)—CH₂—CH₃ |
| B-374 | CH(CH₂—CH₃)—C(CH₃)═CH—CH₃ |
| B-375 | CH(CH₂—CH₃)—CH(CH₃)—CH═CH₂ |
| B-376 | C(CH₃)(CH═CH₂)—CH₂—CH₂—CH₃ |
| B-377 | C(CH₃)(CH₂—CH₃)—CH═CH—CH₃ |
| B-378 | C(CH₃)(CH₂—CH₃)—CH₂—CH═CH₂ |
| B-379 | C[═C(CH₃)—CH₃]—CH₂—CH₂—CH₃ |
| B-380 | CH[C(═CH₂)—CH₃]—CH₂—CH₂—CH₃ |
| B-381 | C(i-C₃H₇)═CH—CH₂—CH₃ |
| B-382 | CH(i-C₃H₇)—CH═CH—CH₃ |
| B-383 | CH(i-C₃H₇)—CH₂—CH═CH₂ |
| B-384 | C(═CH—CH₃)—C(CH₃)₃ |
| B-385 | CH(CH═CH₂)—C(CH₃)₃ |
| B-386 | C(CH₃)(CH═CH₂)CH(CH₃)—CH₃ |
| B-387 | C(CH₃)(CH₂—CH₃)C(═CH₂)—CH₃ |
| B-388 | 2-CH₃-cyclohex-1-enyl |
| B-389 | [2-(═CH₂)]-c-C₆H₉ |
| B-390 | 2-CH₃-cyclohex-2-enyl |
| B-391 | 2-CH₃-cyclohex-3-enyl |
| B-392 | 2-CH₃-cyclohex-4-enyl |
| B-393 | 2-CH₃-cyclohex-5-enyl |
| B-394 | 2-CH₃-cyclohex-6-enyl |
| B-395 | 3-CH₃-cyclohex-1-enyl |
| B-396 | 3-CH₃-cyclohex-2-enyl |
| B-397 | [3-(═CH₂)]-c-C₆H₉ |
| B-398 | 3-CH₃-cyclohex-3-enyl |
| B-399 | 3-CH₃-cyclohex-4-enyl |
| B-400 | 3-CH₃-cyclohex-5-enyl |
| B-401 | 3-CH₃-cyclohex-6-enyl |
| B-402 | 4-CH₃-cyclohex-1-enyl |
| B-403 | 4-CH₃-cyclohex-2-enyl |
| B-404 | 4-CH₃-cyclohex-3-enyl |
| B-405 | [4-(═CH₂)]-c-C₆H₉ |

The compounds of the general formula I can be prepared by various routes analogously to processes, known per se, of the prior art for preparing substituted pyrimidines.

The compounds of the formula I can be obtained, for example, from appropriately substituted pyrimidine compounds of the formula II by nucleophilic substitution according to the synthesis shown in Scheme 1:

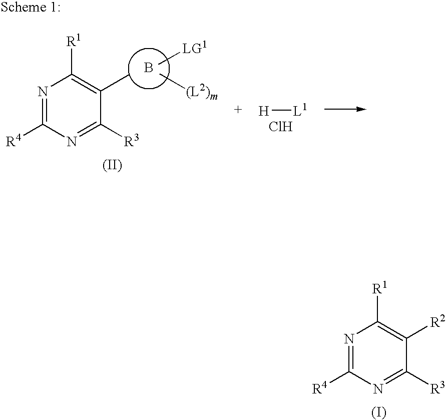

In Scheme 1, R¹, R³, R⁴, L¹ and L² have the meanings given above, m is 0, 1, 2, 3 or 4, LG¹ is a nucleophilically exchangeable group, such as halogen, for example fluorine, and

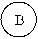

is phenyl or a 5- or 6-membered heteroaromatic radical, where the heteroaromatic radical contains 1, 2, 3 or 4 heteroatoms selected from the group consisting of O, S and N as ring members.

The reaction of II with III is carried out, for example, according to the method described in WO 20005/030775 and is advantageously carried out in the presence of strong bases. Suitable bases are, for example, alkali metal hydroxides, such as sodium hydroxide or potassium hydroxide, alkali metal carbonates, such as sodium carbonate or potassium carbonate, alkaline earth metal carbonates, such as calcium carbonate or magnesium carbonate, or alkali metal hydrides, such as lithium hydride or sodium hydride. The reaction can be carried out in the presence of a solvent. Suitable solvents are aprotic solvents, for example N,N-disubstituted amides, such as N,N-dimethyl-formamide, N,N-dimethylacetamide or N-methylpyrrolidone, sulfoxides, such as dimethyl sulfoxide, or ethers, such as diethyl ether, diisopropyl ether, tert-butyl ether, 1,2-dimethoxyethane, tetrahydrofuran, dioxane or anisole. The reaction is usually carried out at temperatures in the range of from 0° C. to the boiling point of the solvent.

If T in group L¹ is OH or a primary or secondary amino group, it is advantageous to protect the hydroxyl group or the amino group. A suitable protective group for the hydroxyl group is, for example, the benzyl group which, if appropriate, carries a methoxy group in the 4-position of the phenyl ring. The protective group for the hydroxyl group can be removed, for example, by catalytic hydrogenolysis or with the aid of 2,3-dichloro-5,6-dicyano-1,4-benzoquinone (DDQ). A suitable protective group for primary and secondary amino groups is, for example, the tert-butoxycarbonyl group (Boc), which is usually removed using trifluoroacetic acid or p-toluenesulfonic acid.

5-Phenylpyrimidines of the formula II are known from the literature and described, for example, in EP 407899, WO 01/68614, WO 02/074753, WO 03/070721, WO 03/043993, WO 2004/103978, WO 2005/12261, WO 2005/019187 and WO 2005/070899 and in the literature cited therein, the entire contents of which is incorporated herein by way of reference. Compounds II not described in these publications can be prepared analogously to the processes described therein.

5-Hetarylpyrimidines of the formula II are likewise known from the literature and described, for example, in WO 01/68614, WO 2006/029867, WO 2006/005571 and EP 06006255.1 and in the literature cited therein, the entire contents of which is incorporated herein by way of reference. Compounds II not described in these publications can be prepared analogously to the processes described therein.

Compounds of the formula III are generally commercially available or can be prepared by processes known from the literature.

Alternatively, compounds of the formula I in which L¹ is a group attached via oxygen can be obtained according to the process described in Scheme 2.

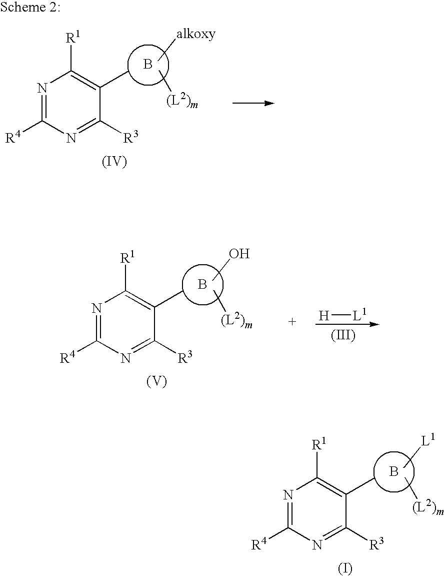

In Scheme 2, R¹, R³, R⁴ and L² are as defined above, m is 0, 1, 2, 3 or 4 and

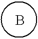

is phenyl or a 5- or 6-membered heteroaromatic radical, where the heteroaromatic radical contains 1, 2, 3 or 4 heteroatoms selected from the group consisting of O, S and N as ring members.

In a first step, the compound IV is reacted with a Lewis acid such as aluminum trichloride or iron(II) chloride, which gives the phenolic compound V. The ether cleavage is usually carried out in an organic solvent, for example in an aromatic hydrocarbon, such as benzene, toluene or xylene. Group L¹ is introduced by nucleophilic substitution of the hydroxyl group under basic conditions, as described in Scheme 1.

Compounds of the formula IV are known from the publications mentioned above.

Compounds of the formula I in which L¹ is a group attached via carbon can be prepared in an advantageous manner from compounds V. Initially, the hydroxyl compound V is reacted with trifluoromethanesulfonic anhydride, which gives a trifluoro-methanesulfonate VI; and this is followed by reaction with an aminoalkylboronic acid. This route is shown in Scheme 3.

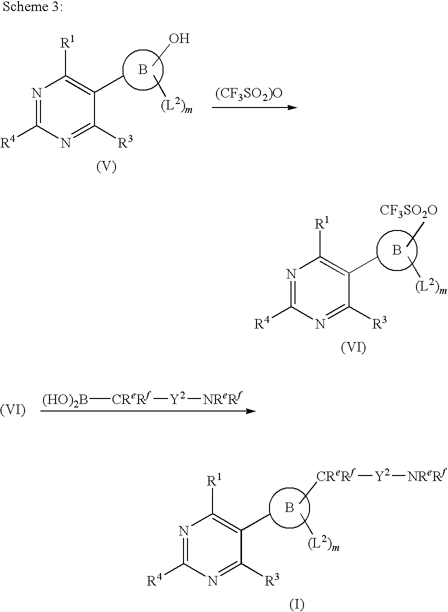

In Scheme 3, R¹, R³, R⁴, R^{e}, R^{f}, Y² and L² are as defined above, m is 0, 1, 2, 3 or 4 and

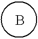

is phenyl or a 5- or 6-membered heteroaromatic radical, where the heteroaromatic radical contains 1, 2, 3 or 4 heteroatoms selected from the group consisting of O, S and N as ring members.

Compounds of the formula I in which L¹ is a group attached via nitrogen can be prepared in an advantageous manner from precursors whose group

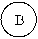

carries an amino group which, if appropriate, can be obtained from the corresponding nitro-substituted compounds by reduction.

Compounds of the formula I in which Z in R⁶ is not hydrogen can also be prepared from hydroxy- or mercaptotriazolopyrimidines of the formula I.

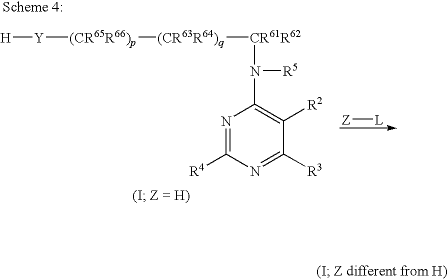

In Scheme 4, R², R³, R⁴, R⁵, R⁶¹ to R⁶⁶, Y, p and q have the meanings mentioned above.

The 4-hydroxy or mercaptoaminopyrimidine of the formula I (Z=H) is reacted with an alkylating or acylating agent Z-L (Z≠H), where L is a nucleophilically removable group. Usually, halides, in particular chlorides or bromides, carboxylic anhydrides, for example acetic anhydride, or carbonyl chlorides, for example acetyl chloride, are employed. Carboxylic acids are generally used in combination with coupling agents, such as, for example, dicyclohexylcarbodiimide, or with strong acids, such as HCl. The reaction conditions suitable for the etherification or esterification are generally known to the person skilled in the art and described, for example, in Organikum, VEB Deutscher Verlag der Wissenschaften, Berlin (1981), the entire contents of which is incorporated herein by way of reference.

Compounds of the formula I in which R¹ is OR⁷ can also be prepared by the route shown in Scheme 5.

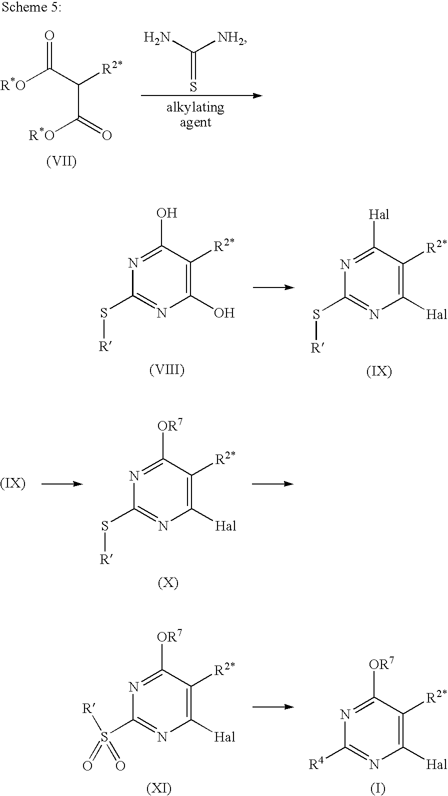

In Scheme 5, R⁷ has the meanings given above, R* and R′ independently of one another are alkyl, preferably C₁-C₆-alkyl. Hal is halogen, preferably chlorine or bromine. R²* is R² or a precursor of R². Here and below, a precursor of R² is to be understood as meaning a group of R² which does not carry a substituent L¹. It is self-evident that the conversion of the group R^{2′} into a group R² can be carried out at any stage of the synthesis of the compounds of the formula I. If appropriate, it may be required to protect the hydroxyl group or the amino group in L¹. For suitable protective groups, reference is made to what was said above.

The malonic ester VII can be reacted with thiourea and an alkylating agent or with S-alkylisothiourea to give the dihydroxyl compound VII. Suitable alkylating agents are, for example, C₁-C₆-alkyl halides, preferably alkyl bromides and alkyl chlorides, di-C₁-C₆-alkyl sulfates or C₁-C₆-alkyl phenylsulfonates. The reaction is usually carried out in the presence of a solvent which is inert under the given reaction conditions. The compound VII is then converted with a halogenating agent [HAL] into the dihalocompounds of the formula IX. The halogenating agent employed is advantageously a phosphorus oxyhalide or a phosphorus(V) halide, such as phosphorus pentachloride, phosphorus oxybromide or phosphorus oxychloride, or a mixture of phosphorus oxychloride with phosphorus pentachloride. If appropriate, a hydrohalide of a tertiary amine, for example triethylamine hydrochloride, may be added as co-catalyst. This reaction of VII with the halogenating agent is usually carried out at from 0° C. to 150° C., preferably at from 80° C. to 125° C. (cf. also EP-A-770615). The reaction can be carried out neat or in an inert solvent, for example a halogenated hydrocarbon, such as dichloromethane, dichloroethane, or an aromatic hydrocarbon, such as, for example, toluene, xylene and the like, or in a mixture of the solvents mentioned above.

The compounds X can be obtained by reacting the compounds IX with an alcohol R⁷OH. Such reactions are known in principle, for example from JACS, 69,1947, 1204f. The reaction is generally carried out in the presence of a base. Suitable bases are alkali metal hydrides, such as sodium hydride or potassium hydride, alkali metal alkoxides or alkaline earth metal alkoxides, such as sodium t-butoxide or potassium t-butoxide, tertiary amines, such as triethylamine or pyridine. Alternatively, the alcohol R⁷OH can initially be reacted with an alkali metal, preferably sodium, with formation of the corresponding alkoxide. The reaction is carried out in excess alcohol or in an inert solvent, such as carboxamides.

Compounds XI can be prepared, for example, by oxidation of the thioethers X. Suitable oxidizing agents are, for example, hydrogen peroxide, selenium dioxide (cf. WO 02/88127) or organic carboxylic acids, such as 3-chloroperbenzoic acid. The oxidation is preferably carried out at from 10 to 50° C. in the presence of protic or aprotic solvents (cf. B. Kor. Chem. Soc., Vol. 16, pp. 489-492 (1995); Z. Chem., Vol. 17, p. 63 (1977)).

If the radical R⁴ in compounds of the formula I is a radical which can be introduced nucleophilically, the compound of the formula I is prepared by reacting the sulfone of the formula XI with compounds R⁴—H. In general, the reaction is carried out under basic conditions. For practical reasons, the alkali metal, alkaline earth metal or ammonium salt of the compound R⁴—H can be employed directly. Alternatively, it is possible to add bases. This reaction is typically carried out under the conditions of a nucleophilic substitution; usually at from 0 to 200° C., preferably at from 10 to 150° C. If appropriate, it may be advantageous to carry out the reaction in the presence of a phase-transfer catalyst, for example 18-crown-6. The reaction is usually carried out in the presence of a dipolar aprotic solvent, such as an N,N-dialkylated carboxamide, for example N,N-dimethylformamide, a cyclic ether, for example tetrahydrofuran, or a carbonitrile, such as acetonitrile (cf. DE-A 39 01 084; Chimia, Vol. 50, pp. 525-530 (1996); Khim. Geterotsikl. Soedin, Vol. 12, pp. 1696-1697 (1998)).

In general, the compounds X¹ and R⁴—H are employed in approximately stoichiometric amounts. However, it may be advantageous to use the nucleophile of the formula R⁴—H in excess, for example in excess of up to 10-fold, in particular up to 3-fold, based on the compound XI.

In general, the reaction is carried out in the presence of a base which may be employed in equimolar amounts or else in excess. Suitable bases are alkali metal carbonates and alkali metal bicarbonates, for example sodium carbonate and sodium bicarbonate, nitrogen bases, such as triethylamine, tributylamine and pyridine, alkali metal alkoxides, such as sodium methoxide or potassium tert-butoxide, alkali metal amides, such as sodium amide, or alkali metal hydrides, such as lithium hydride or sodium hydride.

Suitable solvents are halogenated hydrocarbons, ethers, such as diethyl ether, diisopropyl ether, tert-butyl ether, 1,2-dimethoxyethane, dioxane, anisole and tetrahydrofuran, and also dimethyl sulfoxide, N,N-dialkylated carboxamides, such as dimethylformamide or dimethylacetamide. Particular preference is given to using ethanol, dichloromethane, acetonitrile or tetrahydrofuran. It is also possible to use mixtures of the solvents mentioned.

(Het)arylmalonates of the formula VII can be prepared from (het)aryl compounds of the formula XIII by reaction with one or two equivalents of a carbonic ester or a chloroformate (compound XIII) in the presence of a strong base (see Scheme 6).

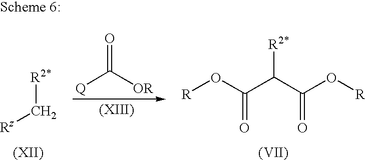

In Scheme 6, R^{z} is hydrogen or a C₁-C₄-alkoxycarbonyl group. Q is halogen or C₁-C₄-alkoxy, in particular methoxy or ethoxy. R²* has the meanings given above and R is C₁-C₄-alkyl. The person skilled in the art will realize that, in the case of R^{z}═H, at least 2 equivalents of the compound XIII have to be used to achieve complete conversion of XII.

The reaction shown in Scheme 6 is usually carried out in the presence of strong bases. If R^{z} is hydrogen, the base employed is usually an alkali metal amide, such as sodium amide or lithium diisopropylamide, or an organolithium compound, such as phenyllithium or butyllithium. In this case, the base is employed in at least equimolar amounts, based on the compound XII, to achieve complete conversion. If R^{z} is an alkoxycarbonyl group, the base used is preferably an alkali metal alkoxide, for example sodium ethoxide or potassium ethoxide, sodium butoxide or potassium butoxide, sodium methoxide or potassium methoxide. For R^{z}═H, the reaction of XII with XIII can be carried out in one step or in two separate steps, where in the latter case the compound VII in which R^{z} is an alkoxycarbonyl group is obtained as intermediate. Besides, the reaction of XII with XIII can be carried out analogously to the method described in J. Med. Chem. 25,1982, p. 745.

Moreover, malonates of the formula VII can be prepared in an advantageous manner by reacting appropriate bromo(het)aryl compounds Br—R²* with dialkyl malonates under Cu(I) catalysis (cf. Chemistry Letters, pp. 367-370, 1981; EP-A-1002788).

Compounds of the formula I in which R¹ is NR⁵R⁶ can also be prepared by the route shown in Scheme 7.

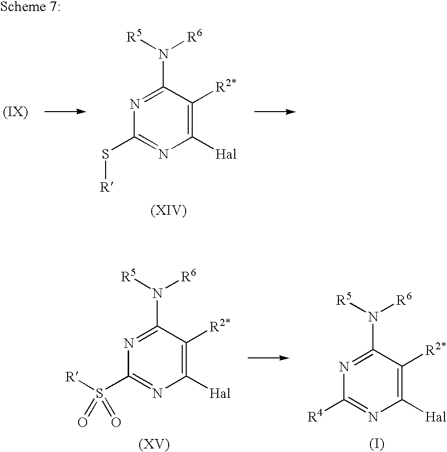

In Scheme 7, Hal, R⁴, R⁵, R⁶, R⁷ and R²* have the meanings given above.

The reaction of IX with an amine HNR⁵R⁶ is usually carried out in an inert solvent, such as ethers, for example dioxane, tetrahydrofuran or diethyl ether, halogenated hydrocarbons, such as dichloromethane, aromatic hydrocarbons, for example toluene, or carboxylic esters, such as ethyl acetate [cf. WO 98/46608]. If appropriate, it may be advantageous to carry out the reaction in the presence of a base, such as tertiary amines, for example triethylamine, or inorganic bases, such as alkali metal carbonates or alkaline earth metal carbonates or alkali metal bicarbonates or alkaline earth metal bicarbonates; it is also possible for excess amine to serve as base. The conversion of the compounds XIV initially into the compound XV and then into the compound I is carried out as shown in Scheme 5 for the conversion of the compound X into the compound XI followed by conversion into the compound I.

Amines of the formula HNR⁵R⁶ are known from the literature, can be prepared by known methods or are commercially available.

Compounds of the formula I in which R¹ is not NR⁵R⁶, OR⁷ or SR⁸ can also be prepared by the process shown in Scheme 8.

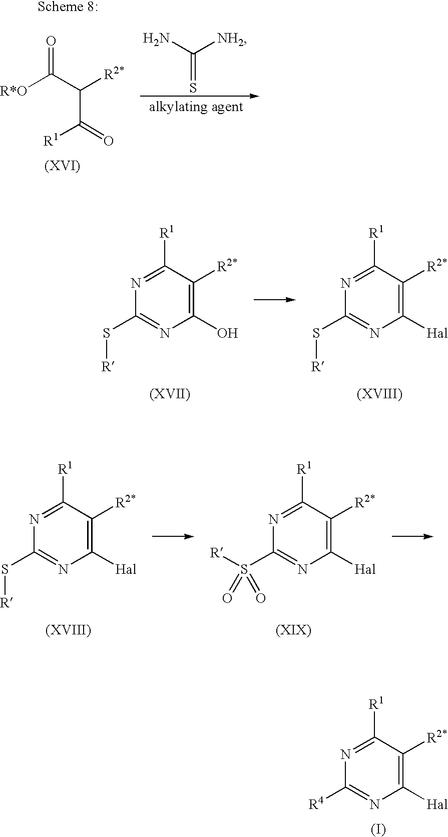

In Scheme 8, R* is alkyl, preferably C₁-C₆-alkyl. Hal is halogen, preferably chlorine or bromine. R²* is R² or a precursor of R². The reactions shown in Scheme 8 can be carried out analogously to the reactions illustrated in Scheme 5.

Compounds of the formula XVI can be prepared analogously to standard processes in the sense of a mixed ester condensation from the corresponding (hetaryl)acetic esters by reaction with the appropriate aliphatic alkyl C₂-C₅-carboxylates, such as ethyl acetate, ethyl propionate, ethyl butyrate or ethyl valerate, or with a reactive derivative thereof, for example an acid chloride or an acid anhydride, in the presence of a strong base, for example an alkoxide, an alkali metal amide or an organolithium compound, for example analogously to the methods described in J. Chem. Soc. Perkin Trans. 1967, 767 or in Eur. J. Org. Chem. 2002, p. 3986.

Compounds of the formula I in which R¹ has a meaning different from NR⁵R⁶, OR⁷ and SR⁸ can be prepared by condensing the thiourea analogously to the preparation of compounds XVII (see Scheme 8) with 1,3-dicarbonyl compounds XX.

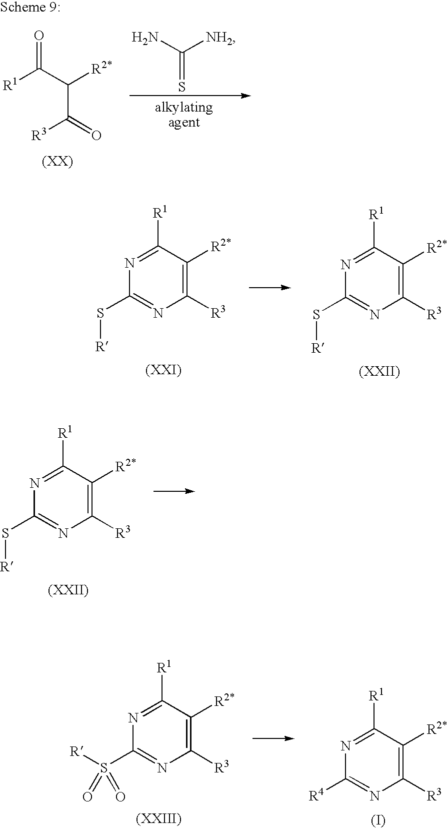

In Scheme 9, R¹ and R³ independently of one another are alkyl. R²* is R² or a precursor of R². The reaction shown in Scheme 9 can be carried out analogously to the reactions illustrated in Scheme 5.

Compounds of the formula I in which R³ is cyano, C₁-C₈-alkoxy, C₁-C₈-alkylthio, C₁-C₈-haloalkoxy or C₁-C₈-haloalkylthio can also be obtained in an advantageous manner by reacting compounds of the formula I in which R³ is halogen, preferably chlorine, with compounds M¹-R^{3′} (hereinbelow also compounds of the formula XXIV). Depending on the group R^{3′} to be introduced, the compounds of the formula XX are inorganic cyanides, alkoxides, thiolates or haloalkoxylates. The reaction is advantageously carried out in an inert solvent. The cation M¹ in formula XXIV is of little importance; for practical reasons preference is usually given to ammonium salts, tetraalkylammonium salts, such as tetramethylammonium salts or tetraethylammonium salts, or alkali metal or alkaline earth metal salts (Scheme 10).

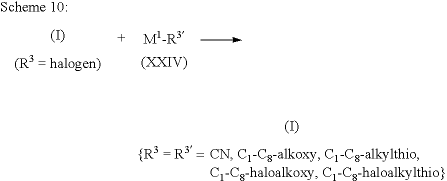

The reaction temperature is usually from 0 to 120° C., preferably from 10 to 40° C. [cf. J. Heterocycl. Chem., Vol. 12, pp. 861-863 (1975)].

Suitable solvents include ethers, dioxane, diethyl ether, methyl tert-butyl ether and, preferably, tetrahydrofuran, halogenated hydrocarbons, such as dichloromethane or dichloroethane, aromatic hydrocarbons such as toluene, and mixtures thereof.

Compounds of the formula I in which R³ is C₁-C₈-alkyl, C₁-C₈-haloalkyl, C₂-C₈-alkenyl, C₂-C₈-haloalkenyl, C₂-C₈-alkynyl or C₂-C₈-haloalkynyl can be prepared in an advantageous manner by reacting compounds of the formula I in which R³ is halogen with organometallic compounds R^{3a}—Mt in which R^{3a} is C₁-C₈-alkyl, C₁-C₈-haloalkyl, C₂-C₈-alkenyl, C₂-C₈-haloalkenyl, C₂-C₈-alkynyl or C₂-C₈-haloalkynyl and Mt is lithium, magnesium or zinc. The reaction is preferably carried out in the presence of catalytic or, in particular, at least equimolar amounts of transition metal salts and/or transition metal compounds, in particular in the presence of Cu salts, such as Cu(I) halides and especially Cu(I) iodide. In general, the reaction is carried out in an inert organic solvent, for example one of the ethers mentioned above, in particular tetrahydrofuran, an aliphatic or cycloaliphatic hydrocarbon, such as hexane, cyclohexane and the like, an aromatic hydrocarbon, such as toluene, or in a mixture of these solvents. The temperatures required for this purpose are in the range of from −100 to +100° C. and especially in the range of from −80° C. to +40° C. Suitable processes are known, for example from the prior art cited at the outset or from WO 03/004465.

Compounds of the formula I in which R⁴ is cyano are useful intermediates for preparing further compounds of the formula I.

Compounds of the formula I in which R⁴ is a derivatized carboxylic acid radical such as C(═O)OR^{a}, C(═O)NR^{a}R^{b}, C(═NOR^{c})NR^{a}R^{b}, C(═O)NR^{a}—NR^{d}R^{b}, C(═N—NR^{c}R^{d})NR^{a}R^{b}, C(═O)R^{c}, CR^{a}R^{b}—OR^{c}, CR^{a}R^{b}—NR^{c}R^{d} can be obtained in an advantageous manner from the compounds of the formula I in which R⁴ is cyano by standard processes for derivatizing CN groups, where R^{a}, R^{b}, R^{c} and R^{d} have the meanings given above.

Compounds of the formula I in which R⁴ is —C(═O)NR^{a}R^{b} can be obtained from compounds of the formula I in which R⁴ is cyano by hydrolysis to the carboxylic acids (where R⁴=—COOH) under acidic or basic conditions and amidation with amines HNR^{a}R^{b}.

By oximation with hydroxylamine or substituted hydroxylamines H₂N—OR^{c} under basic conditions, the amides of the formula I (where R⁴=—CONR^{a}R^{b}) afford the compounds of the formula I in which R⁴ is C(═NOR^{c})NR^{a}R^{b} (cf. U.S. Pat. No. 4,876,252). The substituted hydroxylamines can be used as free base or, preferably, in the form of their acid addition salts. For practical reasons, the halides, such as chlorides, or the sulfates are particularly suitable.

Compounds of the formula I in which R⁴ is —C(═N—NR^{c}R^{d})NR^{a}R^{b} can be prepared in an advantageous manner from the corresponding cyano compounds II by reaction with H₂N—NR^{c}R^{d} to give the corresponding compounds of the formula I where R⁴═C(═N—NR^{a}R^{b})NH₂. The compounds obtained in this manner can be mono- or dialkylated, which gives compounds of the formula I where R⁴=—C(═N—NR^{c}R^{d})NR^{a}R^{b} in which R^{a} and/or R^{b} are different from hydrogen. For suitable alkylation processes, reference is made to what was said above.

Compounds of the formula I in which R⁴ is —C(═O)R^{c} can be obtained from the corresponding cyano compounds I by reaction with Grignard reagents R^{c}-Mg-Hal in which Hal is a halogen atom, in particular chlorine or bromine. This reaction is advantageously carried out under the conditions known from J. Heterocycl. Chem. 1994, Vol. 31(4), p. 1041.

Compounds of the formula I in which R⁴ is —CR^{a}R^{b}—OR^{c} can be obtained from the corresponding ketones in which R⁴ is —C(═O)R^{c} by reaction with Grignard reagents R^{a}R^{b}-Mg-Hal* in which Hal* is a halogen atom, in particular chlorine or bromine, and, if appropriate, subsequent alkylation.

Compounds of the formula I in which R⁴ is —CH₂—OR^{c} can be obtained from the corresponding ketones in which R⁴ is —C(═O)R^{c} by reduction with a metal hydride, for example lithium aluminum hydride, and, if appropriate, subsequent alkylation.

Compounds of the formula I in which R⁴ is —C(═O)OR^{a} can be obtained by esterification of the compounds II (R⁴=—COOH) under acidic or basic conditions.

Compounds of the formula I in which R⁴ is —C(═S)NR^{a}R^{b} can be obtained from compounds of the formula I in which R⁴ is CN by reaction with hydrogen sulfide and, if appropriate, subsequent mono- or dialkylation at the amide nitrogen.

Compounds of the formula I in which R⁴ is a heterocyclic substituent can also be introduced, for example, via the construction of the pyrimidine ring. To this end, a corresponding heterocyclic amidine, which is known to the person skilled in the art or can be prepared from the corresponding heterocyclic nitriles, is reacted with a malonic ester to give the pyrimidine ring (see also WO 2003/070721).

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature.

If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally however not required since in some cases the individual isomers can be interconverted during work-up for use or during application (for example under the action of light, acids or bases). Such conversions may also take place after application, for example, in the case of the treatment of plants, in the treated plants, or in the harmful fungus to be controlled.

The compounds I are suitable for use as fungicides. They have excellent activity against a broad spectrum of phytopathogenic fungi, in particular from the class of the Ascomycetes, Deuteromycetes, Basidiomycetes and Peronosporomycetes (syn. Oomycetes). Some of them are systemically active and can be used in crop protection as foliar fungicides, as fungicides for seed dressing and as soil fungicides.

They are particularly important for the control of a large number of fungi on various crop plants, such as wheat, rye, barley, oats, rice, corn, grass, bananas, cotton, soybeans, coffee, sugarcane, grapevines, fruit and ornamental plants and vegetables, such as cucumbers, beans, tomatoes, potatoes and cucurbits, and also on the seeds of these plants.

The compounds I are particularly suitable for controlling the plant diseases mentioned below:

The compounds I are suitable for controlling Alternaria species on vegetables, rapeseed, sugarbeet and fruit and rice, such as, for example, A. solani or A. alfternata on potatoes and tomatoes.

The compounds I are suitable for controlling Aphanomyces species on sugarbeet and vegetables.

The compounds I are suitable for controlling Ascochyta species on cereals and vegetables.

The compounds I are suitable for controlling Bipolaris and Drechslera species on corn, cereals, rice and lawns, such as, for example, D. maydis on corn.

The compounds I are suitable for controlling Blumeria graminis (powdery mildew) on cereals.

The compounds I are suitable for controlling Botrytis cinerea (gray mold) on strawberries, vegetables, flowers and grapevines.

The compounds I are suitable for controlling Bremia lactucae on lettuce.

The compounds I are suitable for controlling Cercospora species on corn, soybeans, rice and sugarbeet.

The compounds I are suitable for controlling Cochliobolus species on corn, cereals, rice, such as, for example, Cochliobolus sativus on cereals, Cochliobolus miyabeanus on rice.

The compounds I are suitable for controlling Colletotricum species on soybeans and cotton.

The compounds I are suitable for controlling Drechslera species, Pyrenophora species on corn, cereals, rice and lawns, such as, for example, D. teres on barley or D. friftici-repentis on wheat.

The compounds I are suitable for controlling Esca on grapevines, caused by Phaeoacremonium chlamydosporium, Ph. Aleophilum, and Formitipora punctata (syn. Phellinus punctatus).

The compounds I are suitable for controlling Exserohilum species on corn.

The compounds I are suitable for controlling Erysiphe cichoracearum and Sphaerotheca fuliginea on cucumbers.

The compounds I are suitable for controlling Fusarium and Verticillium species on various plants, such as, for example, F. graminearum or F. culmorum on cereals or F. oxysporum on a large number of plants, such as, for example, tomatoes.

The compounds I are suitable for controlling Gaeumanomyces graminis on cereals.

The compounds I are suitable for controlling Gibberella species on cereals and rice (for example Gibberella fujikuroi on rice).

The compounds I are suitable for controlling Grainstaining complexon rice.

The compounds I are suitable for controlling Helminthosporium species on corn and rice.

The compounds I are suitable for controlling Michrodochium nivale on cereals.

The compounds I are suitable for controlling Mycosphaerella species on cereals, bananas and peanuts, such as, for example, M. graminicola on wheat or M. fijiensis on bananas.

The compounds I are suitable for controlling Peronospora species on cabbage and bulbous plants, such as, for example, P. brassicae on cabbage or P. destructoron onions.

The compounds I are suitable for controlling Phakopsara pachyrhizi and Phakopsara meibomiae on soybeans.

The compounds I are suitable for controlling Phomopsis species on soybeans and sunflowers.

The compounds I are suitable for controlling Phytophthora infestans on potatoes and tomatoes.

The compounds I are suitable for controlling Phytophthora species on various plants, such as, for example, P. capsicion bell peppers.

The compounds I are suitable for controlling Plasmopara vifticola on grapevines.

The compounds I are suitable for controlling Podosphaera leucotricha on apples.

The compounds I are suitable for controlling Pseudocercosporella herpotrichoides on cereals.

The compounds I are suitable for controlling Pseudoperonospora on various plants, such as, for example, P. cubensis on cucumbers or P. humilion hops.

The compounds I are suitable for controlling Puccinia species on various plants, such as, for example, P. trifticina, P. striformins, P. hordei or P. graminis on cereals, or P. asparagi on asparagus.

The compounds I are suitable for controlling Pyricularia oryzae, Corticium sasakii, Sarocladium oryzae, S. attenuatum, Entyloma oryzae on rice.

The compounds I are suitable for controlling Pyricularia grisea on lawns and cereals.

The compounds I are suitable for controlling Pythium spp. on lawns, rice, corn, cotton, rapeseed, sunflowers, sugarbeet, vegetables and other plants, such as, for example, P. ultiumum on various plants, P. aphanidermatumon lawns.

The compounds I are suitable for controlling Rhizoctonia species on cotton, rice, potatoes, lawns, corn, rapeseed, sugarbeet, vegetables and on various plants, such as, for example, R. solani on beet and various plants.

The compounds I are suitable for controlling Rhynchosporium secalis on barley, rye and triticale.

The compounds I are suitable for controlling Sclerotinia species on rapeseed and sunflowers.

The compounds I are suitable for controlling Septoria tritici and Stagonospora nodorum on wheat.

The compounds I are suitable for controlling Erysiphe (syn. Uncinula) necator on grapevines.

The compounds I are suitable for controlling Setospaeria species on corn and lawns.

The compounds I are suitable for controlling Sphacelotheca reilinia on corn.

The compounds I are suitable for controlling Thievaliopsis species on soybeans and cotton.

The compounds I are suitable for controlling Tilletia species on cereals.

The compounds I are suitable for controlling Ustilago species on cereals, corn and sugarcane, such as, for example, U. maydis on corn.

The compounds I are suitable for controlling Venturia species (scab) on apples and pears, such as, for example, V. inaequalis on apples.

In addition, the compounds according to the invention can also be used in crops which, owing to breeding including genetic engineering, are tolerant to attack by insects or fungi.

The compounds I are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds according to the invention and/or their agriculturally acceptable salts are employed by treating the fungi or the plants, seeds or materials to be protected against fungal attack or the soil with a fungicidally effective amount of the active compounds. Application can be both before and after the infection of the materials, plants or seeds by the fungi.

Accordingly, the invention furthermore provides a method for controlling phytopathogenic fungi wherein the fungi or the materials, plants, the soil or seed to be protected against fungal attack are/is treated with an effective amount of at least one compound I according to the invention and/or an agriculturally acceptable salt thereof.

The invention furthermore provides a composition for controlling phytopathogenic fungi, which composition comprises at least one compound according to the invention and/or an agriculturally acceptable salt thereof and at least one solid or liquid carrier.

The fungicidal compositions generally comprise between 0.1 and 95% by weight, preferably between 0.5 and 90% by weight, of active compound.

When employed in crop protection, the application rates are, depending on the kind of effect desired, between 0.01 and 2.0 kg of active compound per ha.

In seed treatment, the amounts of active compound required are generally from 1 to 1000 g/100 kg of seed, preferably from 5 to 100 g/100 kg of seed.

When used in the protection of materials or stored products, the active compound application rates depend on the kind of application area and on the desired effect. Amounts typically applied in the protection of materials are, for example, from 0.001 g to 2 kg, preferably from 0.005 g to 1 kg, of active compound per cubic meter of treated material.

The compounds of the formula I can be present in different crystal modifications which may differ in their biological activity. They are likewise subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water. Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations:

1. Products for dilution with water

### A Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content of the concentrate is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

### 2. Products to be Applied Undiluted

### I Dusts (DP, DS)

5 parts by weight of the active compounds are ground finely and mixed intimately with 95 parts by weight of finely divided kaolin. This gives a dustable product with an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

### K ULV Solutions (UL)

10 parts by weight of the active compounds are dissolved in 90 parts by weight of an organic solvent, for example xylene. This gives a product with an active compound content of 10% by weight to be applied undiluted.

Water-soluble concentrates (LS), suspensions (FS), dusts (DS), water-dispersible and water-soluble powders (WS, SS), emulsions (ES), emulsifiable concentrates (EC) and gel formulations (GF) are usually used for the treatment of seed. These formulations can be applied to the seed in undiluted or, preferably, diluted form. The application can be carried out before sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetters, tackifiers, dispersants or emulsifiers and, if appropriate, solvents or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 800; and sodium dioctylsulfosuccinate, for example Leophen RA®.

The compounds according to the invention in the application form as fungicides can also be present together with other active compounds, for example with herbicides, insecticides, growth regulators, fungicides or else with fertilizers. When mixing the compounds according to the invention or the compositions comprising them with one or more further active compounds, in particular fungicides, it is in many cases possible, for example, to widen the activity spectrum or to prevent the development of resistance. In many cases, synergistic effects are obtained.

The present invention furthermore provides a combination of at least one compound according to the invention and/or an agriculturally acceptable salt thereof and at least one further fungicidal, insecticidal, herbicidal and/or growth-regulating active compound.

The following list of fungicides with which the compounds according to the invention can be applied together is meant to illustrate the possible combinations, but not to limit them:

strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethyl-phenyloxymethylene)phenyl)-3-methoxyacrylate;

carboxamides
carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoro-methyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chloro-phenyl)prop-2-ynyloxy]-3-methoxyphenyl)ethyl)-2-methanesulfonylamino-3-methyl-butyramide, N-(2-(4-[3-(4-chlorophenyl)prop-2-ynyloxy]-3-methoxyphenyl)ethyl)-2-ethanesulfonylamino-3-methylbutyramide;
   azoles
triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;
   nitrogenous heterocyclyl compounds
pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propyl-chromen-4-one, N,N-dimethyl-3-(3-bromo-6-fluoro-2-methylindole-1-sulfonyl)-[1,2,4]triazole-1-sulfonamide;
   carbamates and dithiocarbamates
dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;
   other fungicides
guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

Accordingly, the present invention furthermore relates to the compositions listed in Table C, where a row of Table C corresponds in each case to a fungicidal composition comprising a compound of the formula I (component 1), which is preferably one of the compounds described herein as being preferred, and the respective further active compound (component 2) stated in the row in question. According to one embodiment of the invention, component 1 in each row of Table C is in each case one of the compounds of the formula I specifically individualized in Tables 1 to 22848.

TABLE C

| No. | Component 1 | Component 2 |
|---|---|---|
| C-1 | a compound of the formula I | azoxystrobin |
| C-2 | a compound of the formula I | dimoxystrobin |
| C-3 | a compound of the formula I | enestroburin |
| C-4 | a compound of the formula I | fluoxastrobin |
| C-5 | a compound of the formula I | kresoxim-methyl |
| C-6 | a compound of the formula I | metominostrobin |
| C-7 | a compound of the formula I | picoxystrobin |
| C-8 | a compound of the formula I | pyraclostrobin |
| C-9 | a compound of the formula I | trifloxystrobin |
| C-10 | a compound of the formula I | orysastrobin |
| C-11 | a compound of the formula I | methyl (2-chloro-5-[1-(3-methylbenzyloxy- |
| | | imino)ethyl]benzyl)carbamate |
| C-12 | a compound of the formula I | methyl (2-chloro-5-[1-(6-methylpyridin-2-yl- |
| | | methoxyimino)ethyl]benzyl)carbamate |
| C-13 | a compound of the formula I | methyl 2-(ortho-(2,5-dimethylphenyloxy- |
| | | methylene)phenyl)-3-methoxyacrylate |
| C-14 | a compound of the formula I | benalaxyl |
| C-15 | a compound of the formula I | benodanil |
| C-16 | a compound of the formula I | boscalid |
| C-17 | a compound of the formula I | carboxin |
| C-18 | a compound of the formula I | mepronil |
| C-19 | a compound of the formula I | fenfuram |
| C-20 | a compound of the formula I | fenhexamid |
| C-21 | a compound of the formula I | flutolanil |
| C-22 | a compound of the formula I | furametpyr |
| C-23 | a compound of the formula I | metalaxyl |
| C-24 | a compound of the formula I | ofurace |
| C-25 | a compound of the formula I | oxadixyl |
| C-26 | a compound of the formula I | oxycarboxin |
| C-27 | a compound of the formula I | penthiopyrad |
| C-28 | a compound of the formula I | thifluzamide |
| C-29 | a compound of the formula I | tiadinil |
| C-30 | a compound of the formula I | N-(4′-bromobiphenyl-2-yl)-4-difluoromethyl-2- |
| | | methylthiazole-5-carboxamide |
| C-31 | a compound of the formula I | N-(4′-trifluoromethylbiphenyl-2-yl)-4-di- |
| | | fluoromethyl-2-methylthiazole-5-carboxamide |
| C-32 | a compound of the formula I | N-(4′-chloro-3′-fluorobiphenyl-2-yl)-4-di- |
| | | fluoromethyl-2-methylthiazole-5-carboxamide |
| C-33 | a compound of the formula I | N-(3′,4′-dichloro-4-fluorobiphenyl-2-yl)-3-di- |
| | | fluoromethyl-1-methylpyrazole-4-carboxamide |
| C-34 | a compound of the formula I | N-(3′,4′-dichloro-5-fluorobiphenyl-2-yl)-3-di- |
| | | fluoromethyl-1-methylpyrazole-4-carboxamide |
| C-35 | a compound of the formula I | N-(2-cyanophenyl)-3,4-dichloroisothiazole-5- |
| | | carboxamide |
| C-36 | a compound of the formula I | dimethomorph |
| C-37 | a compound of the formula I | flumorph |
| C-38 | a compound of the formula I | flumetover |
| C-39 | a compound of the formula I | fluopicolide (picobenzamid) |
| C-40 | a compound of the formula I | zoxamide |
| C-41 | a compound of the formula I | carpropamid |
| C-42 | a compound of the formula I | diclocymet |
| C-43 | a compound of the formula I | mandipropamid |
| C-44 | a compound of the formula I | N-(2-(4-[3-(4-chlorophenyl)prop-2-ynyloxy]-3- |
| | | methoxyphenyl)ethyl)-2-methanesulfonyl- |
| | | amino-3-methylbutyramide |
| C-45 | a compound of the formula I | N-(2-(4-[3-(4-Chlorophenyl)prop-2-ynyloxy]-3- |
| | | methoxyphenyl)ethyl)-2-ethanesulfonylamino- |
| | | 3-methylbutyramide |
| C-46 | a compound of the formula I | bitertanol |
| C-47 | a compound of the formula I | bromuconazole |
| C-48 | a compound of the formula I | cyproconazole |
| C-49 | a compound of the formula I | difenoconazole |
| C-50 | a compound of the formula I | diniconazole |
| C-51 | a compound of the formula I | enilconazole |
| C-52 | a compound of the formula I | epoxiconazole |
| C-53 | a compound of the formula I | fenbuconazole |
| C-54 | a compound of the formula I | flusilazole |
| C-55 | a compound of the formula I | fluquinconazole |
| C-56 | a compound of the formula I | flutriafol |
| C-57 | a compound of the formula I | hexaconazol |
| C-58 | a compound of the formula I | imibenconazole |
| C-59 | a compound of the formula I | ipconazole |
| C-60 | a compound of the formula I | metconazol |
| C-61 | a compound of the formula I | myclobutanil |
| C-62 | a compound of the formula I | penconazole |
| C-63 | a compound of the formula I | propiconazole |
| C-64 | a compound of the formula I | prothioconazole |
| C-65 | a compound of the formula I | simeconazole |
| C-66 | a compound of the formula I | tebuconazole |
| C-67 | a compound of the formula I | tetraconazole |
| C-68 | a compound of the formula I | triadimenol |
| C-69 | a compound of the formula I | triadimefon |
| C-70 | a compound of the formula I | triticonazole |
| C-71 | a compound of the formula I | cyazofamid |
| C-72 | a compound of the formula I | imazalil |
| C-73 | a compound of the formula I | pefurazoate |
| C-74 | a compound of the formula I | prochloraz |
| C-75 | a compound of the formula I | triflumizole |
| C-76 | a compound of the formula I | benomyl |
| C-77 | a compound of the formula I | carbendazim |
| C-78 | a compound of the formula I | fuberidazole |
| C-79 | a compound of the formula I | thiabendazole |
| C-80 | a compound of the formula I | ethaboxam |
| C-81 | a compound of the formula I | etridiazole |
| C-82 | a compound of the formula I | hymexazole |
| C-83 | a compound of the formula I | fluazinam |
| C-84 | a compound of the formula I | pyrifenox |
| C-85 | a compound of the formula I | 3-[5-(4-chlorophenyl)-2,3-dimethylisoxazo- |
| | | lidin-3-yl]pyridine |
| C-86 | a compound of the formula I | bupirimate |
| C-87 | a compound of the formula I | cyprodinil |
| C-88 | a compound of the formula I | ferimzone |
| C-89 | a compound of the formula I | fenarimol |
| C-90 | a compound of the formula I | mepanipyrim |
| C-91 | a compound of the formula I | nuarimol |
| C-92 | a compound of the formula I | pyrimethanil |
| C-93 | a compound of the formula I | triforine |
| C-94 | a compound of the formula I | fludioxonil |
| C-95 | a compound of the formula I | fenpiclonil |
| C-96 | a compound of the formula I | aldimorph |
| C-97 | a compound of the formula I | dodemorph |
| C-98 | a compound of the formula I | fenpropimorph |
| C-99 | a compound of the formula I | tridemorph |
| C-100 | a compound of the formula I | iprodione |
| C-101 | a compound of the formula I | procymidone |
| C-102 | a compound of the formula I | vinclozolin |
| C-103 | a compound of the formula I | acibenzolar-S-methyl |
| C-104 | a compound of the formula I | anilazin |
| C-105 | a compound of the formula I | captan |
| C-106 | a compound of the formula I | captafol |
| C-107 | a compound of the formula I | dazomet |
| C-108 | a compound of the formula I | diclomezine |
| C-109 | a compound of the formula I | fenoxanil |
| C-110 | a compound of the formula I | folpet |
| C-111 | a compound of the formula I | fenpropidin |
| C-112 | a compound of the formula I | famoxadone |
| C-113 | a compound of the formula I | fenamidone |
| C-114 | a compound of the formula I | octhilinone |
| C-115 | a compound of the formula I | probenazole |
| C-116 | a compound of the formula I | proquinazid |
| C-117 | a compound of the formula I | pyroquilon |
| C-118 | a compound of the formula I | quinoxyfen |
| C-119 | a compound of the formula I | tricyclazole |
| C-120 | a compound of the formula I | 5-chloro-7-(4-methylpiperidin-1-yl)-6-(2,4,6- |
| | | trifluorophenyl)-[1,2,4]triazolo[1,5-a]pyrimidine |
| C-121 | a compound of the formula I | 2-butoxy-6-iodo-3-propylchromene-4-one |
| C-122 | a compound of the formula I | N,N-dimethyl-3-(3-bromo-6-fluoro-2-methyl- |
| | | indole-1-sulfonyl)-[1,2,4]triazole-1-sulfon- |
| | | amide |
| C-123 | a compound of the formula I | ferbam |
| C-124 | a compound of the formula I | mancozeb |
| C-125 | a compound of the formula I | maneb |
| C-126 | a compound of the formula I | metiram |
| C-127 | a compound of the formula I | metam |
| C-128 | a compound of the formula I | propineb |
| C-129 | a compound of the formula I | thiram |
| C-130 | a compound of the formula I | zineb |
| C-131 | a compound of the formula I | ziram |
| C-132 | a compound of the formula I | diethofencarb |
| C-133 | a compound of the formula I | flubenthiavalicarb |
| C-134 | a compound of the formula I | iprovalicarb |
| C-135 | a compound of the formula I | propamocarb |
| C-136 | a compound of the formula I | methyl 3-(4-chlorophenyl)-3-(2-isopropoxy- |
| | | carbonylamino-3-methylbutyrylamino)- |
| | | propionate |
| C-137 | a compound of the formula I | 4-fluorophenyl N-(1-(1-(4-cyanophenyl)- |
| | | ethanesulfonyl)but-2-yl)carbamate |
| C-138 | a compound of the formula I | dodine |
| C-139 | a compound of the formula I | iminoctadine |
| C-140 | a compound of the formula I | guazatine |
| C-141 | a compound of the formula I | kasugamycin |
| C-142 | a compound of the formula I | polyoxine |
| C-143 | a compound of the formula I | streptomycin |
| C-144 | a compound of the formula I | validamycin A |
| C-145 | a compound of the formula I | fentin salts |
| C-146 | a compound of the formula I | isoprothiolane |
| C-147 | a compound of the formula I | dithianon |
| C-148 | a compound of the formula I | edifenphos |
| C-149 | a compound of the formula I | fosetyl |
| C-150 | a compound of the formula I | fosetyl-aluminum |
| C-151 | a compound of the formula I | iprobenfos |
| C-152 | a compound of the formula I | pyrazophos |
| C-153 | a compound of the formula I | tolclofos-methyl |
| C-154 | a compound of the formula I | phosphorous acid and its salts |
| C-155 | a compound of the formula I | thiophanate methyl |
| C-156 | a compound of the formula I | chlorothalonil |
| C-157 | a compound of the formula I | dichlofluanid |
| C-158 | a compound of the formula I | tolylfluanid |
| C-159 | a compound of the formula I | flusulfamide |
| C-160 | a compound of the formula I | phthalide |
| C-161 | a compound of the formula I | hexachlorobenzene |
| C-162 | a compound of the formula I | pencycuron |
| C-163 | a compound of the formula I | quintozene |
| C-164 | a compound of the formula I | binapacryl |
| C-165 | a compound of the formula I | dinocap |
| C-166 | a compound of the formula I | dinobuton |
| C-167 | a compound of the formula I | Bordeaux mixture |
| C-168 | a compound of the formula I | copper acetate |
| C-169 | a compound of the formula I | copper hydroxide |
| C-170 | a compound of the formula I | copper oxychloride |
| C-171 | a compound of the formula I | basic copper sulfate |
| C-172 | a compound of the formula I | sulfur |
| C-173 | a compound of the formula I | spiroxamine |
| C-174 | a compound of the formula I | cyflufenamid |
| C-175 | a compound of the formula I | cymoxanil |
| C-176 | a compound of the formula I | metrafenone |

The active compounds II, mentioned above as component 2, their preparation and their action against harmful fungi are generally known (cf.: http://www.hclrss.demon.co.uk/index.html); they are commercially available. The compounds named according to IUPAC, their preparation and their fungicidal action are likewise known and described, for example, in EP-A 226 917; EP-A 10 28 125; EP-A 10 35 122; EP-A 12 01 648; WO 98/46608; WO 99/24413; WO 03/14103; WO 03/053145; WO 03/066609 and WO 04/049804, the entire contents of which is included herein by way of reference.

The present invention furthermore relates to a pharmaceutical composition comprising at least one pyrimidine compound according to the invention and/or a pharmaceutically acceptable salt thereof and, if appropriate, at least one pharmaceutically acceptable carrier. The invention also relates to the pharmaceutical use of the (novel) pyrimidines of the formula I according to the invention, in particular the (novel) pyrimidines of the formula I described in the above description as being preferred, and/or their pharmaceutically acceptable salts, in particular their use for preparing a medicament for the treatment of cancer.

The pyrimidines of the formula I according to the invention, in particular the pyrimidines of the formula I according to the invention described in the above description as being preferred, and/or their pharmaceutically acceptable salts effectively inhibit the growth and/or the propagation of tumor cells, as can be demonstrated in standard tests with tumor cell lines, such as HeLa, MCF-7 and COLO 205. In particular, the pyrimidines of the formula I according to the invention generally have IC₅₀ values of <10⁻⁶ mol/l (i.e. <1 μM), preferably IC₅₀ values of <10⁻⁷ mol/l (i.e. <100 nM), for cell cycle inhibition in HeLa cells.

The pyrimidines of the formula I according to the invention, in particular the pyrimidines of the formula I according to the invention described in the above description as being preferred, and/or their pharmaceutically acceptable salts are suitable for the treatment, inhibition or control of growth and/or propagation of tumor cells and the disorders associated therewith. Accordingly, they are suitable for cancer therapy in warm-blooded vertebrates, for example mammals and birds, in particular man, but also other mammals, in particular useful and domestic animals, such as dogs, cats, pigs, ruminants (cattle, sheep, goats, bison, etc.), horses and birds, such as chicken, turkey, ducks, geese, guineafowl and the like.

The pyrimidines of the formula I according to the invention, in particular the pyrimidines of the formula I according to the invention described in the above description as being preferred, and/or their pharmaceutically acceptable salts are suitable for the therapy of cancer or cancerous disorders of the following organs: breast, lung, intestine, prostate, skin (melanoma), kidney, bladder, mouth, larynx, oesophagus, stomach, ovaries, pancreas, liver and brain.

In addition to the pyrimidine compound I according to the invention and/or its pharmaceutically acceptable salt, the pharmaceutical compositions according to the invention comprise at least optionally a suitable carrier. Suitable carriers are, for example, solvents, carriers, excipients, binders and the like customarily used for pharmaceutical formulations, which are described below in an exemplary manner for individual types of administration.

The compounds I according to the invention can be administered in a customary manner, for example orally, intravenously, intramuscularly or subcutaneously. For oral administration, the active compound can be mixed, for example, with an inert diluent or with an edible carrier; it can be embedded into a hard or soft gelatin capsule, it can be compressed to tablets or it can be mixed directly with the food/feed. The active compound can be mixed with excipients and administered in the form of indigestible tablets, buccal tablets, pastilles, pills, capsules, suspensions, potions, syrups and the like. Such preparations should contain at least 0.1% of active compound. The composition of the preparation may, of course, vary. It usually comprises from 2 to 60% by weight of active compound, based on the total weight of the preparation in question (dosage unit). Preferred preparations of the compound I according to the invention comprise from 10 to 1000 mg of active compound per oral dosage unit.

The tablets, pastilles, pills, capsules and the like may furthermore comprise the following components: binders, such as traganth, gum arabic, corn starch or gelatin, excipients, such as dicalcium phosphate, disintegrants, such as corn starch, potato starch, alginic acid and the like, glidants, such as magnesium stearate, sweeteners, such as sucrose, lactose or saccharin, and/or flavors, such as peppermint, vanilla and the like. The capsules may furthermore comprise a liquid carrier. Other substances which modify the properties of the dosage unit may also be used. For example, tablets, pills and capsules may be coated with schellack, sugar or mixtures thereof. In addition to the active compound, syrups or potions may also comprise sugar (or other sweeteners), methyl- or propylparaben as preservative, a colorant and/or a flavor. The components of the active compound preparations must, of course, be pharmaceutically pure and nontoxic at the quantities employed. Furthermore, the active compounds can be formulated as preparations with a controlled release of active compound, for example as delayed-release preparations.

The active compounds can also be administered parenterally or intraperitoneally. Solutions or suspensions of the active compounds or their salts can be prepared with water using suitable wetting agents, such as hydroxypropylcellulose. Dispersions can also be prepared using glycerol, liquid polyethylene glycols and mixtures thereof in oils. Frequently, these preparations furthermore comprise a preservative to prevent the growth of microorganisms.

Preparations intended for injections comprise sterile aqueous solutions and dispersions and also sterile powders for preparing sterile solutions and dispersions. The preparation has to be sufficiently liquid for injection. It has to be stable under the preparation and storage conditions and it has to be protected against contamination by microorganisms. The carrier may be a solvent or a dispersion medium, for example, water, ethanol, a polyol (for example glycerol, propylene glycol or liquid polyethylene glycol), a mixture thereof and/or a vegetable oil.

### EXAMPLES

### 1.) Synthesis of compounds I

The syntheses were carried out analogously to the processes described in WO 2003/043993.

1.1) Synthesis of the compound I.A.1 (=compound of the formula I.A,

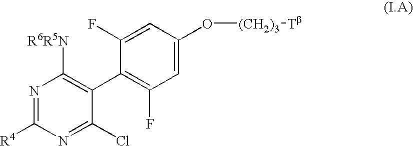

in which T^{β} is NHCH₃, R⁴ is —C(═N—OCH₃)—NH₂ and R⁵ and R⁶ together are —(CH₂)₂—CH(CH₃)—(CH₂)₂—

### a) 4-Chloro-2-cyano-5-(2,6-difluoro-4-hydroxyphenyl)-6-(4-methylpiperidin-1-yl)-pyrimidine

At 5° C., trimethylammonium chloride (1.3 g, 13.5 mmol) was added a little at a time to a solution of aluminum chloride (3.6 g, 27.1 mmol) in toluene (18 ml). The two-phase mixture was allowed to warm to room temperature and then stirred for another 1 h. 4-Chloro-2-cyano-5-(2,6-difluoro-4-methoxyphenyl)-6-(4-methylpiperidin-1-yl)-pyrimidine (1.9 g, 4.5 mmol) was added a little at a time, and the mixture was heated at 100° C. for 2.5 h. The reaction mixture was then added to a saturated aqueous sodium bicarbonate solution on crushed ice, the phases were separated and the aqueous phase was extracted three times with in each case 50 ml of ethyl acetate. The combined organic phases were washed twice with in each case 20 ml of saturated sodium chloride solution. After removal of the solvent under reduced pressure, the crude product was purified by flash chromatography (silica gel; acetonitrile:water 60:40), which gave the title compound in the form of a colorless oil (700 mg; 42% of theory).

### b) 4-Chloro-2-cyano-5-(2,6-difluoro-4-(3-(N-(tert-butyloxycarbonyl)amino)propoxy)-phenyl)-6-(4-methylpiperidin-1-yl)-pyrimidine

Triphenylphosphine (270 mg, 1.03 mmol), N-(3-hydroxypropyl)-N-(tert-butyloxy-carbonyl)amine (182 mg, 0.96 mmol) and a solution of diisopropyl azodicarboxylate (208 mg, 1.03 mmol) in 1 ml of THF were added successively to a solution of the product obtained in step a) (250 mg, 0.69 mmol) in 2 ml of THF. The mixture was stirred for 2 h and then concentrated. The crude product was purified by flash chromatography (silica gel; acetonitrile:water 60:40 to 90:10), which gave the title compound in the form of a pale yellow oil (220 mg; 57% of theory).

### c) 4-Chloro-2-(N-methoxyamidine)-5-(2,6-difluoro-4-(3-(N-(tert-butyloxycarbonyl)-amino)propoxy)phenyl)-6-(4-methylpiperidin-1-yl)-pyrimidine

Sodium methoxide (4.03 g, 0.02 mmol) was added to a solution of the product obtained in step b) (120 mg, 0.22 mmol) in 2.5 ml of methanol, and the mixture was stirred overnight. O-Methylhydroxylamine hydrochloride (22 mg, 0.27 mmol) was then added, and the mixture was stirred overnight. The solvent was removed under reduced pressure, and 10 ml of methyl tert-butyl ether were added to the residue. The solution was washed twice with in each case 5 ml of water, dried over sodium sulfate and concentrated under reduced pressure, which gave the title product (100 mg, 73% of theory) in the form of a pale yellow oil.

### d) Compound I.A.1

A mixture of the compound obtained in step c) (50 mg, 0.09 mmol) and Amberlyst 15 (H⁺) (200 mg) in 2 ml of methylene chloride was shaken at room temperature for 16 h. The resin was filtered off and washed twice with in each case 10 ml of methylene chloride. After addition of 10 ml of a 10M solution of ammonia in methanol, the mixture was stirred for a further 2 h. The resin was filtered again and washed twice with in each case 10 ml of methanol. The filtrate was concentrated under reduced pressure (bath temperature <30° C.), which gave the title compound (30 mg, 72% of theory) in the form of a pale yellow oil.

The following compounds of the formula I.A listed in Table 1 were prepared analogously:

TABLE 1

| | |
|---|---|
| | (I.A) |
| 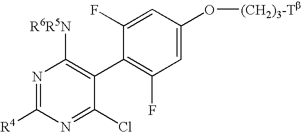 | |

| Ex. | | | | | | M.p. |
|---|---|---|---|---|---|---|
| No. | R⁴ | R⁵ | R⁶ | T^{β} | LCMS | [° C.] |
| 1. | —C(═N—OCH₃)—NH₂ | —(CH₂)₂—CH(CH₃)—CH₂)₂— | | NHCH₃ | 483 | |
| 2. | —C(═N—OCH₃)—NH₂ | —(CH₂)₂—CH(CH₃)—CH₂)₂— | | N(CH₃)₂ | 497 | |
| 3. | —C(═N—OCH₃)—NH₂ | —(CH₂)₂—CH(CH₃)—CH₂)₂— | | N(CH₃)—CO—O-t-Bu | 583 | |
| 4. | —C(═N—OCH₃)—NH₂ | CH(CH₃)CF₃ | H | NHCH₃ | 497 | |
| 5. | —C(═N—OCH₃)—NH₂ | —(CH₂)₆— | | NHCH₃ | 483 | |
| 6. | —C(═N—OCH₃)—NH₂ | —(CH₂)₆— | | N(CH₃)—CO—O-t-Bu | 583 | |
| 7. | —C(═N—OCH₃)—NH₂ | CH(CH₃)C(CH₃)₃ | H | OCH₃ | 486 | |
| 8. | —C(═N—OCH₃)—NH₂ | CH(CH₃)C(CH₃)₃ | H | OCH₃ | 472 | |
| 9. | —C(═N—OCH₃)—NH₂ | —(CH₂)₂—CH(CH₃)—CH₂)₂— | | OCH₃ | 437 | |
| 10. | pyrazol-1-yl | —(CH₂)₂—CH(CH₃)—CH₂)₂— | | NHCH₃ | 477 | |
| 11. | pyrazol-1-yl | —(CH₂)₂—CH(CH₃)—CH₂)₂— | | N(CH₃)—CO—O-t-Bu | 577 | |
| 12. | pyrazol-1-yl | —(CH₂)₂—CH(CH₃)—CH₂)₂— | | O—(4-Cl-pyrimidin-6-yl) | 576 | |
| 13. | pyrazol-1-yl | —(CH₂)₆— | | NHCH₃ | 477 | |
| 14. | pyrazol-1-yl | —(CH₂)₆— | | N(CH₃)—CO—O-t-Bu | 577 | |
| 15. | pyrazol-1-yl | CH(CH₃)C(CH₃)₃ | H | OCH₃ | 480 | |
| 16. | pyrazol-1-yl | CH(CH₃)CH(CH₃)₂ | H | OCH₃ | 466 | 134-135 |
| 17. | [1,2,3]-(1H)-triazol-1-yl | CH(CH₃)CH(CH₃)₂ | H | OCH₃ | 467 | |
| 18. | [1,2,3]-(1H)-triazol-1-yl | CH(CH₃)C(CH₃)₃ | H | OCH₃ | 481 | |
| 19. | [1,2,3]-(2H)-triazol-2-yl | CH(CH₃)CH(CH₃)₂ | H | OCH₃ | 467 | |
| 20. | [1,2,3]-(2H)-triazol-2-yl | CH(CH₃)C(CH₃)₃ | | OCH₃ | 481 | |
| 21. | [1,2,4]-(1H)-triazol-1-yl | —(CH₂)₂—CH(CH₃)—CH₂)₂— | | NHCH₃ | 478 | |
| 22. | [1,2,4]-(1H)-triazol-1-yl | —(CH₂)₂—CH(CH₃)—CH₂)₂— | | N(CH₃)—CO—O-t-Bu | 578 | |
| 23. | [1,2,4]-(1H)-triazol-1-yl | —(CH₂)₂—CH(CH₃)—CH₂)₂— | | O—(4-Cl-pyrimidin-6-yl) | 577 | |
| 24. | [1,2,4]-(1H)-triazol-1-yl | CH(CH₃)C(CH₃)₃ | H | OCH₃ | 481 | |
| 25. | [1,2,4]-(1H)-triazol-1-yl | CH(CH₃)CH(CH₃)₂ | H | OCH₃ | 467 | |
| 26. | CN | —(CH₂)₂—CH(CH₃)—CH₂)₂— | | NHCH₃ | 436 | |
| 27. | CN | —(CH₂)₂—CH(CH₃)—CH₂)₂— | | N(CH₃)₂ | 450 | |
| 28. | CN | —(CH₂)₂—CH(CH₃)—CH₂)₂— | | N(CH₃)—CO—O-t-Bu | 536 | |
| 29. | CN | —(CH₂)₂—CH(CH₃)—CH₂)₂— | | pyrrolidin-2-on-1-yl | 490 | |
| 30. | CN | —(CH₂)₂—CH(CH₃)—CH₂)₂— | | [1,2,4]-triazol-1-yl | 474 | |
| 31. | CN | —(CH₂)₂—CH(CH₃)—CH₂)₂— | | OCH₃ | 437 | |
| 32. | CN | —(CH₂)₂—CH(CH₃)—CH₂)₂— | | O—(4-Cl-pyrimidin-6-yl) | 535 | |
| 33. | CN | —(CH₂)₆— | | NHCH₃ | 436 | |
| 34. | CN | —(CH₂)₆— | | N(CH₃)—CO—O-t-Bu | 536 | |
| 35. | CN | CH(CH₃)C(CH₃)₃ | H | OCH₃ | 439 | |
| 36. | CN | CH(CH₃)CH(CH₃)₂ | H | OCH₃ | 425 | 99-100 |
| 37. | CN | CH(CH₃)C(CH₃)₃ | H | Cl | 444 | |
| 38. | CN | CH(CH₃)CH(CH₃)₂ | H | Cl | 430 | 121-122 |

| | | | | |
|---|---|---|---|---|
| 39. | CO-(para-tolyl) | —(CH₂)₆— | NHCH₃ | 529 |
| 40. | CO-(para-tolyl) | —(CH₂)₆— | N(CH₃)—CO—O-t-Bu | 629 |

### 2.) Fungicidal Activity

### Microtiter Tests

The active compounds were formulated separately as a stock solution in dimethyl sulfoxide having a concentration of 10 000 ppm.

Use example 1—activity against the gray mold pathogen Botrytis cinerea in the microtiter test:

The stock solution was pipetted into a microtiter plate (MTP) and diluted to the stated active compound concentration using a malt-based aqueous nutrient medium for fungi. An aqueous spore suspension of Botrytis cinerea was then added. The plates were placed in a water vapor-saturated chamber at temperatures of 18° C. Using an absorption photometer, the MTPs were measured at 405 nm on day 7 after the inoculation. The measured parameters were compared to the growth of the active compound-free control variant (=100%) and the fungus- and active compound-free blank value to determine the relative growth in % of the pathogens in the individual active compounds.

In this test, the samples which had been treated with in each case 125 ppm of the compounds from examples 2, 7, 8, 9, 15, 16, 17, 18, 19, 20, 23, 24, 27, 29, 30, 31, 34 and 35 showed a relative pathogen growth of at most 16%.

Use example 2—activity against the rice blast pathogen Pyricularia oryzae in the microtiter test:

The stock solution was pipetted into a microtiter plate (MTP) and diluted to the stated active compound concentration using a malt-based aqueous nutrient medium for fungi. An aqueous spore suspension of Pyricularia oryzae was then added. The plates were placed in a water vapor-saturated chamber at temperatures of 18° C. Using an absorption photometer, the MTPs were measured at 405 nm on day 7 after the inoculation. The measured parameters were compared to the growth of the active compound-free control variant (=100%) and the fungus- and active compound-free blank value to determine the relative growth in % of the pathogens in the individual active compounds.

In this test, the samples which had been treated with in each case 125 ppm of the compounds from examples 7, 8, 9, 10, 11, 12, 15, 16, 17, 18, 19, 20, 22, 24, 27, 29, 30, 31, 32, 35, 36, 37 and 38 showed a relative pathogen growth of at most 14%.

### 3.) Pharmacological Activity—Cell Cycle Inhibition in Hela Cells

### General Procedure

HeLa B cells were cultivated in DMEM (Life Technologies Cat. No. 21969-035) containing fetal calf serum (FCS, Life Technologies Cat. No. 10270-106) in 180 cm² containers at 37° C., 92% humidity and 7% CO₂.

5×10⁴ cells per well were added into a 24-well plate. After 20 h, the compounds to be tested were added such that the final concentration was 1×10⁻⁶, 3.3×10⁻⁷, 1.1×10⁻⁷, 3.7×10⁻⁸, 1.2×10⁻⁸ and 1×10⁻⁹ M in a final volume of 500 μl. 6 wells contained only DMSO as control. The treated cells were incubated as described above for a further 20 h. The cells were then examined under the microscope for dead cells. The 24-well plate was then centrifuged at 1200 rpm at 20° C., an acceleration in position 7 and the brake position 5 (Eppendorf centrifuge 5804R) for 5 min.

The supernatant was removed and the cells were lysed with 0.5 ml of RNase buffer (10 mM sodium citrate, 0.1% Nonidet NP40, 50 μg/ml RNase, 10 μg/ml propidium iodide) per well. The plates were then incubated at room temperature in the dark for at least 30 min, and the samples were then transferred into FACS tubes. These were measured in an FACS instrument (Beckton Dickinson) with the following settings:

Instrument settings at the FACS Calibur:

Run modus: high

| Parameter | Voltage | Amp gain | Mode |
|---|---|---|---|
| FSC | E01 | 2.5 | lin |
| SSC | 350 | 1 | lin |
| FI 1 | | | |
| FI 2 | 430 | 2 | lin |
| FI 3 | | | |
| FI 2-A | — | 1 | lin |
| FI 2-W | — | 3 | lin |

| | |
|---|---|
| DDM parameter | FI 2 |

The ratio of cells in the G₀/G₁ phase to those in the G₂/M phase was calculated and compared to the value for the control (DMSO). In Table 2 below, the results are stated as IC₅₀ values calculated from the curve of the concentration against the cell cycle ratio; they state the concentration at which 50% of the cells are inhibited in their cell cycle.

Similar tests were also carried out using different cell lines (MCF-7 and COLO 205), where the cells were incubated in the growth media recommended by the American Tissue Culture Collection for the respective cell type.

TABLE 2

| Ex. No. | IC₅₀ [nM] |
|---|---|
| 2 | 27 |
| 7 | 56 |
| 8 | 53 |
| 9 | 38 |
| 11 | 570 |
| 12 | 470 |
| 15 | 32 |
| 16 | 26 |
| 17 | 32 |
| 18 | 29 |
| 19 | 26 |
| 20 | 27 |
| 21 | 18 |
| 22 | 290 |
| 23 | 330 |
| 24 | 42 |
| 25 | 42 |
| 27 | 220 |
| 31 | 120 |
| 32 | 570 |
| 33 | 480 |
| 35 | 190 |
| 36 | 59 |
| 37 | 280 |
| 38 | 330 |
| 39 | 580 |

1-32. (canceled)

33. A method of controlling harmful fungi comprising, treating said fingi or the materials, plants, the soil or seed to be protected against said fungi with an effective amount of at least one compound of formula I 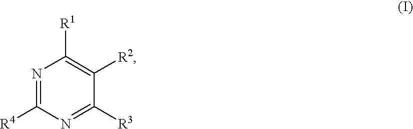
wherein
R¹ is C₁-C₁₀-alkyl, C₂-C₁₀-alkenyl, C₂-C₁₀-alkynyl, C₃-C₁₀-cycloalkyl, C₃-C₁₀-cycloalkenyl, phenyl, naphthyl or a saturated or unsaturated aromatic or non-aromatic 5-, 6-, 7-, 8-, 9- or 10-membered heterocycle, wherein said heterocycle contains 1, 2, 3 or 4 heteroatoms selected from the group consisting of O, S and N as ring members and may furthermore contain one or two CO groups as ring members, where R¹ may be partially or fully halogenated and/or may carry 1, 2, 3 or 4 identical or different substituents L³; or
is a radical of the formula NR⁵R⁶, OR⁷ or SR⁸;
R² is phenyl or a 5- or 6-membered heteroaromatic radical, wherein said heteroaromatic radical contains 1, 2, 3 or 4 heteroatoms selected from the group consisting of O, S and N as ring members, wherein phenyl or said heteroaromatic radical carries a substituent L¹ and optionally 1, 2, 3 or 4 identical or different substituents L²;
R³ is halogen, hydroxyl, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-alkylsulfonyl, C₁-C₄-alkoxy-C₁-C₄-alkyl, cyano-C₁-C₄-alkyl or cyano;
R⁴ is halogen, cyano, hydroxyl, mercapto, N₃, C₁-C₆-alkyl, C₂-C₈-alkenyl, C₂-C₈-alkynyl, C₁-C₆-alkoxy, C₃-C₈-alkenyloxy, C₃-C₈-alkynyloxy, C₁-C₆-alkylthio, C₃-C₈-alkenylthio, C₃-C₈-alkynylthio, C₁-C₆-alkylsulfinyl, C₁-C₆-alkylsulfonyl, hydroxysulfonyl, aminosulfonyl, C₁-C₆-alkylaminosulfonyl, di-C₁-C₆-alkylaminosulfonyl, C₃-C₁₀-cycloalkyl, phenyl, naphthyl, 3-, 4-, 5-, 6-, 7-, 8-, 9- or 10-membered saturated, partially unsaturated or aromatic heterocyclyl having 1, 2, 3 or 4 heteroatoms selected from the group consisting of O, N and S and optionally 1 or 2 carbonyl groups as ring members,
or a radical of the formula —ON(═CR^{a}R^{b}), —NR^{c}N═CR^{a}R^{b}, —NR^{a}R^{b}, —NR^{c}NR^{a}R^{b}, —NR^{a}═CN, —N═OR^{a}; —NR^{c}C(═W)—NR^{a}R^{k}, NR^{a}C(═W)R^{c}, —NNR^{a}R^{b}C(═W)—X —R^{c}, —OC(═W)R^{c}, —O(C═W)NR^{a}R^{b}, —C(═W)R^{c}, C(═W)NR^{a}R^{b}, —C(═W)NR^{a}OR^{b}, —CR^{a}R^{b}—R^{c}, CR^{a}R^{b}—SR^{c}, —CR^{a}R^{b}NR^{c}R^{d}, —CR^{a}R^{b}—C(═W)R^{c}, —C(═W)—NR^{a}—X²—R^{b}, —C(═NX²R^{a})—OR^{b} or —C(═NX²R^{a})—SR^{b},
wherein
W is O, S, NR^{d} or NNR^{d}R^{e};
X¹ is O or NR^{f};
X² is a single bond, —CO—, —CONH—, —COO—, —O—, —NR^{f}—, —CH₂—O—CO— or —CH═CH—(C═O)—, wherein the left part of the divalent radicals is attached to the nitrogen atom;
R^{a}, R^{b}, R^{c}, R^{d}, R^{e}, R^{f} independently of one another are hydrogen, hydroxyl, C₁-C₆-alkyl, C₂-C₈-alkenyl, C₂-C₈-alkynyl, C₁-C₆-alkoxy, C₁-C₄-alkoxy-C₁-C₄-alkyl, C₁-C₆-alkylcarbonyl, C₃-C₆-cycloalkyl, C₃-C₆-cycloalkenyl, C₃-C₆-cycloalkoxy, aryl, aryl-C₁-C₄-alkyl or 5- to 10-membered heterocyclyl having 1, 2, 3 or 4 heteroatoms selected from the group consisting of O, N and S and optionally 1 or 2 carbonyl groups as ring members;
wherein, if R^{a}, R^{b}, R^{c} are attached directly to an oxygen atom, they are not hydroxyl, C₁-C₆-alkoxy or C₃-C₆-cycloalkoxy;
or R^{a} and R^{b} together with the nitrogen atom to which they are attached form a group R^{c}—X¹¹—C(R^{g})═N wherein
R^{g} is independently defined like R^{a} or is halogen or cyano; and
X¹¹ is independently defined like X¹;
or two of the radicals R^{a}, R^{b}, R^{c}, R^{d}, R^{e}, R^{k}, R^{g} together form a C₂-C₄-alkylene group which may be interrupted by an oxygen atom and/or may contain a C—C double bond,
wherein the aliphatic, alicyclic, aromatic and/or heterocyclic groups in R⁴, R^{a}, R^{b}, R^{c}, R^{d}, R^{e}, R^{f} and/or R^{g} may be partially or fully halogenated and/or may have 1, 2 or 3 substituents R^{x}, wherein
R^{x} is cyano, nitro, amino, aminocarbonyl, aminothiocarbonyl, hydroxyl, mercapto, oxo, carboxyl, C₁-C₆-alkyl, C₁-C₆-haloalkyl, C₁-C₆-alkylcarbonyl, C₃-C₆-cycloalkyl, C₃-C₆-halocycloalkyl, C₃-C₆-cycloalkyl-C₁-C₄-alkyl, C₃-C₆-cycloalkenyl, C₁-C₆-alkoxy, C₁-C₆-haloalkoxy, C₁-C₆-alkyloxycarbonyl, C₁-C₆-alkylthio, C₁-C₆-alkylsulfinyl, C₁-C₆-alkylsulfonyl, hydroxysulfonyl, aminosulfonyl, C₁-C₆-alkylaminosulfonyl, di-C₁-C₆-alkylaminosulfonyl, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₁-C₆-alkylcarbonylamino, C₂-C₆-alkenyl, C₂-C₆-alkynyl, C₂-C₆-alkenyloxy, C₃-C₆-alkynyloxy, tri-C₁-C₆-alkylsilyl, aryl, aryloxy, aryl-C₁-C₄-alkyl, aryl-C₁-C₄-alkoxy, 5- or 6-membered saturated, partially unsaturated or aromatic heterocyclyl, 5- or 6-membered saturated, partially unsaturated or aromatic heterocyclyloxy, 5- or 6-membered saturated, partially unsaturated or aromatic heterocyclylcarbonyl, wherein said heterocyclyl radicals in the three last-mentioned groups contain 1, 2, 3 or 4 heteroatoms selected from the group consisting of O, N and S and optionally 1 or 2 carbonyl groups as ring members, —C(═NOR^{α})—OR^{β} or —OC(R^{α})₂—C(R^{β})═NOR^{β},
wherein the cyclic radicals in R^{x} may be unsubstituted or may carry 1, 2 or 3 radicals R^{y}, where
R^{y} is cyano, nitro, halogen, hydroxyl, amino, aminocarbonyl, aminothiocarbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl,
C₃-C₆-cycloalkenyl, phenyl, phenoxy, phenylthio, benzyl, benzyloxy, 5- or 6-membered saturated, partially unsaturated or aromatic heterocyclyl, 5- or 6-membered saturated, partially unsaturated or aromatic heterocyclyloxy, wherein said heterocyclyl radicals in the two last-mentioned groups contain 1, 2, 3 or 4 heteroatoms selected from the group consisting of O, N and S and optionally 1 or 2 carbonyl groups as ring members, or —C(═NOR^{α})—OR^{β}; where
R^{α}, R^{β} independently of one another are hydrogen or C₁-C₆-alkyl;
R⁵ is H, C₁-C₁₀-alkyl, C₂-C₁₀-hydroxyalkyl, C₂-C₁₀-alkenyl, C₂-C₁₀-alkynyl, C₄-C₁₀-alkadienyl, C₃-C₁₀-cycloalkyl, C₁-C₁₀-alkoxy, C₂-C₁₀-alkenyloxy, C₂-C₁₀-alkynyloxy, amino, C₁-C₈-alkylamino, di-(C₁-C₈-alkyl)amino, phenyl, naphthyl or a saturated or unsaturated aromatic or non-aromatic 5- or 6-membered heterocycle which is attached via a carbon atom, wherein said heterocycle contains 1, 2, 3 or 4 heteroatoms selected from the group consisting of O, S and N as ring members and may furthermore contain 1 or 2 CO groups as ring members;
wherein the aliphatic, alicyclic, aromatic and/or heterocyclic groups in R⁵ may be partially or fully halogenated and/or may carry 1, 2, 3 or 4 identical or different substituents R^{a1};
R⁶ is independently defined like R⁵, with the proviso that R⁵ and R⁶ are not both H, or is a group
#-CR⁶¹R⁶²—(CR⁶³R⁶⁴)_{q}—(CR⁶⁵R⁶⁶)ₚ—Y-Z in which
# is the point of attachment to the nitrogen atom;
R⁶¹, R⁶²R⁶³, R⁶⁴, R⁶⁵ and R⁶⁶ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle which contains one, two, three or four heteroatoms from the group consisting of O, N and S; where
R⁶³ with R⁶¹ or R⁶⁶ together with the atoms to which these radicals are attached may also form a five-, six-, seven-, eight-, nine- or ten-membered saturated or partially unsaturated ring which, in addition to carbon atoms, may contain one, two or three heteroatoms from the group consisting of O, N and S as ring members and/or may carry one or more substituents R^{a1};
R⁶¹ with R⁶², R⁶³ with R⁶⁴, R⁶⁵ with R⁶⁶ in each case together may also be oxygen, thus forming carbonyl groups, and form a C₂-C₅-alkylene, C₂-C₅-alkenylene or C₂-C₅-alkynylene chain (which may be interrupted by one, two or three heteroatoms from the group consisting of O, N and S), thus forming spiro groups;
R⁵ and R⁶¹ together with atoms to which they are attached may form a 5-, 6-7-, 8-, 9- or 10-membered saturated or partially unsaturated heterocycle which, in addition to carbon atoms, may contain one, two or three further heteroatoms from the group consisting of O, N and S as ring members;
wherein
the aliphatic, alicyclic, heterocyclic, aromatic and/or heteroaromatic radicals in R⁶¹ to R⁶⁶ in each case independently of one another may be partially or fully halogenated and/or may carry one, two, three or four identical or different groups R^{a1};
each R^{a1} is independently cyano, nitro, hydroxyl, carboxyl, C₁-C₆-alkyl, C₂-C₈-alkenyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C₁-C₆-alkylthio, amino, C₁-C₆-alkylamino, di-(C₁-C₆-alkyl)amino, C(O)R^{π}, C(S)R^{π}, C(O)OR^{π}, C(S)OR^{π}, C(O)SR^{π}, C(S)SR^{π}, C(O)NH₂, C(O)NHR^{π}, C(O)NR^{π}₂, OC(O)OR^{π}, OC(O)NH₂, OC(O)NHR^{π}, OC(O)NR^{π}₁₂, C₁-C₆-alkylene, oxy-C₁-C₄-alkylene, oxy-C₁-C₃-alkyleneoxy, wherein the three last mentioned divalent groups may be attached to the same atom or to adjacent atoms, phenyl, naphthyl or a 5-, 6-, 7-, 8-, 9- or 10-membered saturated, partially unsaturated or aromatic heterocycle which contains one, two, three or four heteroatoms from the group consisting of O, N and S;
each R^{π} is independently C₁-C₈-alkyl, C₃-C₈-alkenyl, C₃-C₈-alkynyl, C₃-C₆-cycloalkyl or C₃-C₆-cycloalkenyl;
wherein the aliphatic, alicyclic, aromatic or heterocyclic groups in the abovementioned groups R^{a1} and R^{π} for their part may be partially or fully halogenated and/or may carry one, two or three groups R^{b1};
each R^{b1} is independently cyano, nitro, hydroxyl, mercapto, amino, carboxyl, C₁-C₆-alkyl, C₂-C₈-alkenyl, C₁-C₆-alkoxy, C₂-C₈-alkenyloxy, C₂-C₈-alkynyloxy, C₁-C₆-alkylthio, C₁-C₆-alkylamino, di-(C₁-C₆-alkyl)amino, formyl, C₁-C₆-alkylcarbonyl, C₁-C₆-alkylsulfonyl, C₁-C₆-alkylsulfinyl, C₁-C₆-alkoxycarbonyl, C₁-C₆-alkylcarbonyloxy, C₁-C₆-alkoxycarbonyloxy, aminocarbonyl, aminothiocarbonyl, C₁-C₆-alkylaminocarbonyl, di-(C₁-C₆-alkyl)aminocarbonyl, C₁-C₆-alkylaminothiocarbonyl, di-(C₁-C₆-alkyl)aminothiocarbonyl, C₃-C₁₀-cycloalkyl, C₃-C₁₀-cycloalkoxy, heterocyclyl, heterocyclyloxy, wherein said heterocyclyl in the two last mentioned radicals is 3- to 10-membered and contains 1, 2, 3 or 4 heteroatoms selected from the group consisting of O, N and S and optionally 1 or 2 carbonyl groups as ring members; aryl, aryloxy, arylthio, aryl-C₁-C₆-alkoxy, aryl-C₁-C₆-alkyl, hetaryl, hetaryloxy or hetarylthio, wherein the aryl radicals contain 6 to 10 ring members and the hetaryl radicals 5 or 6 ring members and 1, 2, 3 or 4 heteroatoms selected from the group consisting of O, N and S, wherein the alicyclic, heterocyclic, aromatic and/or heteroaromatic systems may be partially or fully halogenated and/or substituted by 1, 2, 3, 4 or 5 C₁-C₄-alkyl and/or C₁-C₄-haloalkyl groups;
p is 0, 1, 2, 3, 4 or 5,
q is 0 or 1,
Y is oxygen or sulfur;
Z is hydrogen, carboxyl, formyl, C₁-C₈-alkyl, C₂-C₈-alkenyl, C₂-C₈-alkynyl, C₃-C₆-cycloalkyl, C₃-C₈-cycloalkenyl, C(O)R^{π}, C(O)OR^{π}, C(S)OR^{π}, C(O)SR^{π}, C(S)SR^{π}, C(NR^{A})SR^{π}, C(S)R^{π}, C(NR^{π})NR^{A}R^{B}, C(NR^{π})R^{A}, C(NR^{π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkylene-NR^{A}C(NR^{π})NR^{A}R^{B} C(S)—C₁-C₄-alkylene-NR^{A}C(NR^{π})NR^{A}R^{B}, C(NR^{π})-C₁-C₄-alkylene-NR^{A}C(NR^{π})NR^{A}R^{B}, phenyl, naphthyl, a five-, six-, seven-, eight-, nine- or ten-membered saturated, partially unsaturated or aromatic heterocycle which contains one, two, three or four heteroatoms from the group consisting of O, N and S and which is attached directly or via a carbonyl, thiocarbonyl, C₁-C₄-alkylcarbonyl or C₁-C₄-alkylthiocarbonyl group; where the carbon chains in group Z may be substituted by one or more groups R^{b1};
R^{A} and R^{B} independently of one another are hydrogen, C₂-alkenyl, C₂-alkynyl or one of the groups mentioned under R^{π}; or
R^{A} and R^{B} together with the nitrogen atom to which they are attached or R^{A} and R^{π} together with the carbon atoms and heteroatoms to which they are attached may also form a five- or six-membered saturated, partially unsaturated or aromatic ring which, in addition to carbon atoms, may contain one, two or three further heteroatoms from the group consisting of O, N and S as ring members and/or may carry one or more substituents R^{a1};
or
Z with R⁶⁴ or R⁶⁶ may also form a five- or six-membered saturated or partially unsaturated ring which, in addition to carbon atoms and Y, may contain one or two further heteroatoms from the group consisting of O, N and S as ring members and/or may carry one or more substituents R^{a1};
wherein said group Z may be partially or fully halogenated and/or carry one, two or three groups R^{b1};
or R⁵ and R⁶ together with the nitrogen atom to which they are attached form a saturated or unsaturated aromatic or non-aromatic 5-, 6-, 7- or 8-membered heterocycle, wherein said heterocycle may additionally contain 1, 2 or 3 heteroatoms selected from the group consisting of O, S and N and/or 1 or 2 CO groups as ring members and wherein the heterocycle may carry 1, 2 or 3 substituents selected from the group consisting of halogen, hydroxyl, cyano, nitro, carboxyl, C₁-C₈-alkyl, C₁-C₈-haloalkyl, C₂-C₈-hydroxyalkyl, C₁-C₈-alkoxy, C₁-C₈-haloalkoxy, C₁-C₈-alkylthio, C₁-C₈-haloalkylthio, C₂-C₈-alkenyl, C₂-C₈-haloalkenyl, C₂-C₈-alkenyloxy, C₂-C₈-haloalkenyloxy, C₂-C₈-alkynyl, C₃-C₈-haloalkynyl, C₂-C₈-alkynyloxy, C₃-C₈-haloalkynyloxy, C₃-C₈-cycloalkyl, C₃-C₈-cycloalkoxy, C₃-C₈-cycloalkenyl, C₃-C₈-cycloalkenyloxy, amino, C₁-C₈-alkylamino, di-(C₁-C₈-alkyl)amino, C₁-C₉-alkylcarbonyl, C₁-C₈-haloalkylcarbonyl, C₂-C₈-alkenylcarbonyl, C₂-C₈-haloalkenylcarbonyl, C₂-C₈-alkynylcarbonyl, C₃-C₈-haloalkynylcarbonyl, C₃-C₈-cycloalkylcarbonyl, C₃-C₈-cycloalkenylcarbonyl, C₁-C₈-alkylcarbonyloxy, C₁-C₈-haloalkylcarbonyloxy, C₂-C₈-alkenylcarbonyloxy, C₂-C₈-haloalkenylcarbonyloxy, C₂-C₈-alkynylcarbonyloxy, C₃-C₈-haloalkynylcarbonyloxy, C₃-C₈-cycloalkylcarbonyloxy, C₃-C₈-cycloalkenylcarbonyloxy, C₁-C₈-alkoxycarbonyl, C₁-C₈-haloalkoxycarbonyl, C₂-C₈-alkenyloxycarbonyl, C₂-C₈-haloalkenyloxycarbonyl, C₂-C₈-alkynyloxycarbonyl, C₃-C₈-haloalkynyloxycarbonyl, C₃-C₈-cycloalkoxycarbonyl, cycloalkenyloxycarbonyl, aminocarbonyl, C₁-C₈-alkylaminocarbonyl, di-(C₁-C₈-alkyl)aminocarbonyl, C₁-C₈-alkoxycarbonyloxy, C₁-C₈-haloalkoxycarbonyloxy, C₂-C₈-alkenyloxycarbonyloxy, C₂-C₈-haloalkenyloxycarbonyloxy, C₂-C₈-alkynyloxycarbonyloxy, C₃-C₈-haloalkynyloxycarbonyloxy, C₃-C₈-cycloalkoxycarbonyloxy, cycloalkenyloxycarbonyloxy, aminocarbonyloxy, C₁-C₈-alkylaminocarbonyloxy and di-(C₁-C₈-alkyl)aminocarbonyloxy;
R⁷ and R⁸ independently of one another are hydrogen, C₁-C₁₀-alkyl, C₂-C₁₀-alkenyl, C₂-C₁₀-alkynyl, C₃-C₁₀-cycloalkyl, C₃-C₁₀-cycloalkenyl, phenyl, naphthyl or a saturated or unsaturated aromatic or non-aromatic 5-, 6-, 7-, 8-, 9- or 10-membered heterocycle, wherein said heterocycle contains 1, 2, 3 or 4 heteroatoms selected from the group consisting of O, S and N as ring members and may furthermore contain 1 or 2 CO groups as ring members, wherein the aliphatic, alicyclic, aromatic and/or heterocyclic groups in R⁷ and/or R⁸ may be partially or fully halogenated and/or may carry 1, 2, 3 or 4 identical or different substituents L⁴;
L¹ is a group of the formula —Y¹—Y²-T in which
Y¹ is CR^{h}Rⁱ, C(O)O, C(O)NR^{h}, O, NR^{h} or S(O)ᵣ;
Y² is C₁-C₈-alkylene, C₂-C₈-alkenylene or C₂-C₈-alkynylene, wherein Y² may be interrupted by one, two, three or four heteroatoms from the group consisting of NR^{h}, O and S(O)ᵣ;
r is 0, 1 or 2;
T is halogen, OR^{h}, NR^{h}Rⁱ, C(O)OR^{h}, C(O)NR^{h}Rⁱ, C(NOR^{h})Rⁱ or
T¹-C(=T²)-T³ in which
T¹ is O or NR^{h};
T² is O, S or NR^{h};
T³ is R^{h}, OR^{h}, SR^{h} or NR^{h}Rⁱ;
each R^{h} and Rⁱ is independently H, C₁-C₈-alkyl, C₂-C₈-alkenyl, C₂-C₈-alkynyl, C₃-C₆-cycloalkyl, C₃-C₆-cycloalkenyl, phenyl or a 5- or 6-membered heteroaromatic radical, wherein said heteroaromatic radical contains 1, 2, 3 or 4 heteroatoms selected from the group consisting of O, S and N as ring members, wherein phenyl and the heteroaromatic radical may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy,
or R^{h} and Rⁱ together with the nitrogen atom to which they are attached in the radical NR^{h}Rⁱ form a 5- or 6-membered saturated, partially unsaturated or aromatic heterocycle which may contain 1, 2 or 3 further heteroatoms selected from the group consisting of N, O and S and/or 1 or 2 carbonyl groups as ring members and/or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy;
each L² is independently halogen, hydroxyl, mercapto (SH), cyano, cyanato (OCN), nitro, carboxyl (COOH), C₁-C₁₀-alkyl, C₁-C₁₀-haloalkyl, C₂-C₁₀-hydroxyalkyl, C₁-C₁₀-alkoxy, C₁-C₁₀-haloalkoxy, C₁-C₁₀-alkylthio, C₂-C₁₀-alkenyl, C₂-C₁₀-haloalkenyl, C₂-C₁₀-alkenyloxy, C₂-C₁₀-alkynyl, C₃-C₁₀-haloalkynyl, C₂-C₁₀-alkynyloxy, C₃-C₁₀-cycloalkyl, C₃-C₁₀-cycloalkoxy, C₃-C₁₀-cycloalkyl-C₁-C₄-alkyl, C₃-C₁₀-cycloalkenyl, C₁-C₁₀-alkoxycarbonyl, C₁-C₁₀-haloalkoxycarbonyl, C₂-C₁₀-alkenyloxycarbonyl, C₂-C₁₀-alkynyloxycarbonyl, C₁-C₁₀-alkylcarbonyloxy, C₁-C₁₀-alkenylcarbonyloxy, C₁-C₁₀-alkynylcarbonyloxy, aminocarbonyl, C₁-C₁₀-alkylaminocarbonyl, di-(C₁-C₁₀-alkyl)aminocarbonyl, C₁-C₁₀-alkoximinoalkyl, C₂-C₁₀-alkenyloximinoalkyl, C₂-C₁₀-alkynyloximinoalkyl, formyl, C₁-C₁₀-alkylcarbonyl, C₂-C₁₀-alkenylcarbonyl, C₂-C₁₀-alkynylcarbonyl, C₃-C₆-cycloalkylcarbonyl, NR^{j}R^{k}, NR^{j}—(C═O)—R^{k}, S(═O)ₙA¹, C(═S)A², a group —C(═N—OR^{l})(NR^{m}Rⁿ) or a group —C(═N—NR^{o}R^{p})(NR^{q}R^{r});
wherein
R^{j}, R^{k}, R^{l}, R^{m}, Rⁿ, R^{o}, R^{p}, R^{q}, R^{r} are each independently H, C₁-C₈-alkyl, C₁-C₈-haloalkyl, C₂-C₈-hydroxyalkyl, C₂-C₈-alkenyl, C₂-C₈-haloalkenyl, C₂-C₈-alkynyl, C₃-C₈-cycloalkyl or C₃-C₈-cycloalkenyl; or
R^{m} and Rⁿ, R^{o} and R^{p} and/or R^{q} and R^{r} together with the nitrogen atom to which they are attached form a four-, five- or six-membered saturated or partially unsaturated ring which may carry one, two, three or four substituents independently of one another selected from L⁵;
A¹ is hydrogen, hydroxyl, C₁-C₈-alkyl, amino, C₁-C₈-alkylamino or di-(C₁-C₈-alkyl)amino;
A² is C₂-C₈-alkenyl, C₁-C₈-alkoxy, C₁-C₆-haloalkoxy, C₂-C₁₀-alkenyloxy, C₂-C₁₀-alkynyloxy or one of the groups mentioned under A¹; and
n is 0, 1 or 2;
each L³ is independently defined like L² or is phenyl, naphthyl or a saturated or unsaturated aromatic or non-aromatic 5-, 6-, 7-, 8-, 9- or 10-membered heterocycle, wherein said heterocycle contains 1, 2, 3 or 4 heteroatoms selected from the group consisting of O, S and N as ring members and may furthermore contain one or two CO groups as ring members, wherein the aliphatic, alicyclic, aromatic and heterocyclic groups in L³ for their part may be partially or fully halogenated and/or may carry 1, 2 or 3 substituents L⁴;
each L⁴ is independently cyano, nitro, hydroxyl, mercapto, amino, carboxyl, aminocarbonyl, aminothiocarbonyl, C₁-C₆-alkyl, C₁-C₆-haloalkyl, C₂-C₈-alkenyl, C₄-C₈-alkadienyl, C₂-C₈-alkenyloxy, C₂-C₈-alkynyloxy, C₁-C₆-alkoxy, C₁-C₆-haloalkoxy, C₁-C₆-alkylthio, C₁-C₆-alkylamino, di-(C₁-C₆-alkyl)amino, formyl, C₁-C₆-alkylcarbonyl, C₁-C₆-alkylsulfonyl, C₁-C₆-alkylsulfinyl, C₁-C₆-alkoxycarbonyl, C₁-C₆-alkylcarbonyloxy, C₁-C₆-alkylaminocarbonyl, di-(C₁-C₆-alkyl)aminocarbonyl, C₁-C₆-alkylaminothiocarbonyl, di-(C₁-C₆-alkyl)aminothiocarbonyl, C₃-C₈-cycloalkyl, bicycloalkyl, C₃-C₈-cycloalkoxy, heterocyclyl, heterocyclyloxy, aryl, aryloxy, arylthio, aryl-C₁-C₆-alkoxy or aryl-C₁-C₆-alkyl, wherein the heterocyclyl radicals may be saturated or unsaturated, aromatic or non-aromatic and have 5 to 10 ring members and 1, 2, 3 or 4 heteroatoms selected from the group consisting of O, S and N and optionally one or two carbonyl groups as ring members and wherein the cyclic systems may be partially or fully halogenated and/or substituted by C₁-C₆-alkyl or C₁-C₆-haloalkyl groups; and
each L⁵ is in each case independently selected from the group consisting of hydroxyl, cyano, nitro, C₁-C₈-alkyl, C₁-C₈-haloalkyl, C₂-C₈-hydroxyalkyl, C₁-C₈-alkoxy, C₁-C₈-haloalkoxy, C₁-C₈-alkylthio, C₂-C₈-alkenyl, C₂-C₈-haloalkenyl, C₂-C₈-alkenyloxy, C₂-C₈-alkynyl, C₂-C₈-alkynyloxy, C₃-C₈-cycloalkyl, amino, C₁-C₈-alkylamino and di-(C₁-C₈-alkyl)amino;
and/or the agriculturally acceptable salts thereof
for controlling harmful fungi.

34. The method of claim 33, wherein R⁴ is a radical R^{4a} which is a 3-, 4-, 5-, 6-, 7-, 8-, 9- or 10-membered saturated, partially unsaturated or aromatic heterocyclic ring having 1, 2, 3 or 4 heteroatoms selected from the group consisting of O, N and S and optionally 1 or 2 carbonyl groups as ring members, wherein the heterocyclic ring may be partially or fully halogenated and/or carry 1, 2 or 3 radicals R^{x}.

35. The method of claim 34, wherein said heterocyclic ring is unsubstituted or carries 1 or 2 substituents selected from the group consisting of halogen, C₁-C₄-alkyl, C₁-C₄-haloalkyl, C₁-C₄-alkoxy and C₁-C₄-haloalkoxy.

36. The method of claim 34, wherein R⁴ is a radical R^{4aa} which is a 5- or 6-membered heteroaromatic ring which contains a nitrogen atom and optionally 1 or 2 further heteroatoms selected from the group consisting of O, N and S as ring members.

37. The method of claim 34, wherein R⁴ is a radical R^{4ab} which is a 5- or 6-membered saturated or partially unsaturated heterocyclic ring which contains a nitrogen atom and optionally 1 or 2 further heteroatoms selected from the group consisting of O, N and S and/or one or two carbonyl groups as ring members.

38. The method of claim 33, wherein R⁴ is CN or a radical R^{4b} of the formula ON(═CR^{a}R^{b}), —NR^{c}N═CR^{a}R^{b}, —N═OR^{a}; —NR^{c}C(═W)—NR^{a}R^{b}, —NR^{a}C(═W)R^{c}, —NNR^{a}R^{b}C(═W)—X¹—R^{c}, —OC(═W)R^{c}, —O(C═W)NR^{a}R^{b}, —C(═W)R^{c}, —C(═W)NR^{a}R^{b} C(═W)NR^{a}OR^{b}, —CR^{a}R^{b}—C(═W)R^{c}, —C(═W)—NR^{a}—X²—R^{b}, —C(═NX²R^{a})—OR^{b} or —C(═NX²R^{a})—SR^{b}.

39. The method of claim 38, wherein R⁴ is CN or a radical R^{4ba} of the formula —NR^{a}C(═O)R^{c}, —C(═O)—R^{c}, —C(═O)—OR^{c}, —C(═NR^{d})R^{c}, —C(═NR^{d})—NR^{a}—X²—R^{b} —C(═N—NR^{d}R^{e})—NR^{a}—X²—R^{b}, C(═O)—NR^{a}—X²—R^{b} or —C(═S)—NR^{a}—X²—R^{b},
wherein
X² is a single bond, —CO—, —CONH—, —COO—, —O— or —NR^{f}, wherein the left part of the divalent radicals is attached to the nitrogen atom;
R^{a} is hydrogen, hydroxyl, C₁-C₄-alkyl, C₁-C₄-alkoxy or C₁-C₄-alkylcarbonyl; and
R^{b}, R^{c}, R^{d} and R^{e} independently of one another are hydrogen, hydroxyl, C₁-C₄-alkyl, C₁-C₄-alkoxy or phenyl, where phenyl may carry 1 or 2 substituents selected from the group consisting of halogen, C₁-C₄-alkyl, C₁-C₄-haloalkyl, C₁-C₄-alkoxy and C₁-C₄-haloalkoxy;
wherein, if R^{a}, R^{b}, R^{c} or R^{d} are attached directly to an oxygen atom, they are not hydroxyl or C₁-C₄-alkoxy.

40. The method of claim 39, wherein R⁴ is CN or a radical R^{ba} of the formula —C(═O)—R^{c}, —C(═O)—OR^{c}, —C(═NR^{d})R^{c}, —C(═NR^{d})—NR^{a}R^{b}, —C(═O)—NR^{a}R^{b} or —C(═S)—NR^{a}R^{b},
wherein
R^{a} is hydrogen, hydroxyl, C₁-C₄-alkyl, C₁-C₄-alkoxy or C₁-C₄-alkylcarbonyl; and
R^{b}, R^{c}, R^{d} and R^{e} independently of one another are hydrogen, hydroxyl, C₁-C₄-alkyl, C₁-C₄-alkoxy or phenyl, wherein phenyl may carry 1 or 2 substituents selected from the group consisting of halogen, C₁-C₄-alkyl, C₁-C₄-haloalkyl, C₁-C₄-alkoxy and C₁-C₄-haloalkoxy;
wherein, if R^{a}, R^{b}, R^{c} or R^{d} are attached directly to an oxygen atom, they are not hydroxyl or C₁-C₄-alkoxy.

41. The method of claim 33, wherein R is a radical R^{4c} of the formula —NR^{a}R^{b}, —NR^{c}NR^{a}R^{b}, —NR^{a}—CN, —CR^{a}R^{b}—OR^{c}, —CR^{a}R^{b}—SR^{c} or —CR^{a}R^{b}—NR^{c}R^{d}.

42. The method of claim 33, wherein R¹ is C₁-C₁₀-alkyl, C₂-C₁₀-alkenyl, C₂-C₁₀-alkynyl, C₃-C₁₀-cycloalkyl, C₃-C₁₀-cycloalkenyl, phenyl, naphthyl or a saturated or unsaturated aromatic or non-aromatic 5-, 6-, 7-, 8-, 9- or 10-membered heterocycle, where the heterocycle contains 1, 2, 3 or 4 heteroatoms selected from the group consisting of O, S and N as ring members and may furthermore contain 1 or 2 CO groups as ring members, where R¹ may be partially or fully halogenated and/or may carry 1, 2, 3 or 4 identical or different substituents L³.

43. The method of claim 33, wherein R¹ is NR⁵R⁶.

44. The method of claim 43, wherein
R⁵ is C₁-C₈-alkyl or C₁-C₈-haloalkyl and
R⁶ is H, C₁-C₈-alkyl or C₁-C₈-haloalkyl; or
R⁵ and R⁶ together with the nitrogen atom to which they are attached form a saturated or unsaturated 5-, 6- or 7-membered heterocycle, where the heterocycle may additionally contain a heteroatom or a heteroatom-containing group selected from the group consisting of O, N and NR′″ as ring member, where R′″ is H, C₁-C₈-alkyl, C₁-C₈-haloalkyl or C₂-C₈-hydroxyalkyl and where the heterocycle may carry 1, 2 or 3 substituents selected from the group consisting of halogen, hydroxyl, C₁-C₈-alkyl, C₁-C₈-haloalkyl, C₂-C₈-hydroxyalkyl, C₁-C₈-alkoxy and C₁-C₈-haloalkoxy.

45. The method of claim 43, wherein
R⁵ is H, C₁-C₈-alkyl or C₁-C₈-haloalkyl and R⁶ is a group #-CR⁶R⁶²—(CR⁶³R⁶⁴)_{q}—(CR⁶⁵R⁶⁶)ₚ—Y-Z wherein R⁶¹, R⁶², R⁶³, R⁶⁴, R⁶⁵, R⁶⁶, Y, Z, p and q are as defined in claim 1.

46. The method of claim 43, wherein where neither R⁵ nor R⁶ is H.

47. The method of claim 33, wherein R³ is halogen, C₁-C₈-alkyl, C₁-C₈-haloalkyl, C₁-C₈-alkoxy, C₁-C₈-haloalkoxy or CN.

48. The method of claim 33, wherein R² is phenyl, pyridinyl, pyrimidinyl, pyrazinyl, pyridazinyl, triazinyl, furyl, thienyl, pyrrolyl, pyrazolyl, imidazolyl, oxazolyl, isoxazolyl, thiazolyl, isothiazolyl, triazolyl, oxadiazolyl, thiadiazolyl or tetrazolyl which carries a substituent L¹ and optionally 1 or 2 substituents L²

49. The method of claim 48, wherein R² is phenyl which carries a substituent L¹ and optionally 1 or 2 substituents L².

50. The method of claim 33, wherein L¹ is a radical L¹¹ of the formula
Y^{α1}A^{α}—Y^{α2}ₐ-A^{α}-T^{α}
wherein
A^{α} is C₁-C₄-alkylene;
Y^{α1}, Y^{α2} independently of one another are O, S or NR^{hα};
T^{α} is OR^{hα}, SR^{hα} or NR^{hα}R^{iα};
R^{hα} and R^{iα} independently of one another are H or C₁-C₄-alkyl; and
a is 1, 2, 3 or 4.

51. The method of claim 33, wherein L¹ is a radical L¹² of the formula
Y^{β}-A^{β}-T^{β}
wherein
Y^{β} is CH₂, O, S or NR^{hβ};
A^{β} is C₁-C₈-alkylene;
T^{β} is halogen, OR^{hβ}NR^{hβ}R^{iβ}, NR^{hβ}C(═O)-T^{3β} or OC(═O)-T^{3β};
T^{3β} is R^{hβ}, OR^{hβ} or NR^{hβ}R^{iβ}; and
R^{hβ} and R^{iβ} independently of one another are H, C₁-C₈-alkyl, C₂-C₈-alkenyl, C₂-C₈-alkynyl, C₃-C₆-cycloalkyl, C₃-C₆-cycloalkenyl, phenyl or a 5- or 6-membered heteroaromatic radical, wherein said heteroaromatic radical contains 1, 2, 3 or 4 heteroatoms selected from the group consisting of O, S and N as ring members, wherein phenyl and the heteroaromatic radical may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy, or R^{h} and Rⁱ together with the nitrogen atom to which they are attached in the radical NR^{h}Rⁱ form a 5- or 6-membered saturated, partially unsaturated or aromatic heterocycle which may contain 1, 2 or 3 further heteroatoms selected from the group consisting of N, O and S and/or 1 or 2 carbonyl groups as ring members and/or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy.

52. The method of claim 51, wherein
Y^{β} is O;
A^{β} is C₁-C₄-alkylene;
T^{β} is halogen, OR^{hβ}, NR^{hβR}^{iβ} or NR^{hβ}C(═O)-T^{3β};
T^{3β} is R^{hβ}, OR^{hβ} or NR^{hβ}R^{iβ}; and
R^{hβ} and R^{iβ} independently of one another are H, C₁-C₆-alkyl or a 5- or 6-membered heteroaromatic radical, wherein the heteroaromatic radical contains 1, 2 or 3 heteroatoms selected from the group consisting of O, S and N as ring members or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy,
or R^{h} and Rⁱ together with the nitrogen atom to which they are attached in the radical NR^{h}Rⁱ form a 5- or 6-membered saturated, partially unsaturated or aromatic heterocycle which may contain 1, 2 or 3 further heteroatoms selected from the group consisting of N, O and S and/or 1 or 2 carbonyl groups as ring members and/or may carry 1, 2 or 3 substituents selected from the group consisting of halogen, hydroxyl, C₁-C₄-alkyl, C₁-C₄-haloalkyl, C₁-C₄-alkoxy and C₁-C₄-haloalkoxy.

53. The method of claim 33, wherein L¹ is a radical L¹³ of the formula
—Y^{1γ}-A^{γ}-T^{γ}
in which
Y^{1γ} is —CONR^{hγ} or —COO;
A^{γ} is C₂-C₆-alkylene;
T^{γ} is OR^{hγ}, NR^{hγ}R^{iγ} or OC(═O)-T^{3γ};
T^{3γ} is R^{hγ}, OR^{hγ} or NR^{hγ}R^{iγ}; and
R^{hγ} and R^{iγ} independently of one another are H or C₁-C₄-alkyl.

54. A compound of formula I of claim 33, except for compounds wherein
R¹ is NR⁵R⁶, wherein R⁵ is H and R⁶ is C₃-C₆-haloalkyl, or is C₃-C₁₀-cycloalkyl and simultaneously
R² is phenyl which carries a substituent L¹ of the formula —Y¹—Y²-T in which Y¹ is O, NR^{h} or S, Y² is C₁-C₄-alkylene and T is OR^{h} or NR^{h}Rⁱ and optionally one or two substituents L² selected from the group consisting of halogen,
R³ is halogen and
R⁴ is NR^{a}R^{b}, NR^{a}—CN, phenyl, naphthyl or 5- to 10-membered hetaryl.

55. A compound of formula I of claim 33, wherein R² is a 5- or 6-membered heteroaromatic radical, wherein said heteroaromatic radical contains 1, 2, 3 or 4 heteroatoms selected from the group consisting of O, S and N as ring members, carries a substituent L¹ and optionally 1, 2, 3 or 4 identical or different substituents L².

56. A compound of formula I of claim 33, wherein R² is phenyl or a 5- or 6-membered heteroaromatic radical, wherein said heteroaromatic radical contains 1, 2, 3 or 4 heteroatoms selected from the group consisting of O, S and N as ring members, wherein phenyl or the heteroaromatic radical carries a substituent L¹ and optionally 1, 2, 3 or 4 identical or different substituents L².

57. A compound of formula I of claim 33, wherein R⁴ is a radical of the formula —ON(═CR^{a}R^{b}), —NR^{c}N═CR^{a}R^{b}, —N═OR^{a}; —NR^{c}C(═W)—NR^{a}R^{b}, —NR^{a}C(═W)R^{c}, —NNR^{a}R^{b}C(═W)—X¹—R^{c}, —OC(═W¹)R^{c}, —O(C═W¹)NR^{a}R^{b}, —C(═W)R^{c}, —C(═W)NR^{a}R^{b}, —C(═W)NR^{a}OR^{b}, —CR^{a}R^{b}—C(═W)R^{c}, —C(═W)—NR^{a}—X²—R^{b}, C(═NX²R^{a})—OR^{b} or —C(═NX²R^{a})—SR^{b}.

58. A compound of formula I of claim 33, wherein R⁴ is 3-, 4-, 5-, 6-, 7-, 8-, 9- or 10-membered saturated or partially unsaturated heterocyclyl having 1, 2, 3 or 4 heteroatoms selected from the group consisting of O, N and S and optionally 1 or 2 carbonyl groups as ring members, wherein said heterocyclyl radical may be partially or fully halogenated and/or carry 1, 2 or 3 substituents R^{x}.

59. A compound of formula I of claim 33, wherein R¹ is C₁-C₁₀-alkyl, C₂-C₁₀-alkenyl, C₂-C₁₀-alkynyl, phenyl, naphthyl or a saturated or unsaturated aromatic or non-aromatic 5-, 6-, 7-, 8-, 9- or 10-membered heterocycle, wherein said heterocycle contains 1, 2, 3 or 4 heteroatoms selected from the group consisting of O, S and N as ring members and may furthermore contain 1 or 2 CO groups as ring members, wherein R¹ may be partially or fully halogenated and/or may carry 1, 2, 3 or 4 identical or different substituents L³.

60. A compound of formula I of claim 33, wherein R¹ is a radical of the formula NR⁵R⁶, where R⁵ and R⁶, with the proviso that neither R⁵ nor R⁶ is H.

61. A compound of formula I of claim 33, wherein R¹ is a radical of the formula OR⁷ or SR⁸.

62. A fungicidal composition comprising at least one compound of formula I of claim 54 and/or at least one agriculturally acceptable salt thereof and, optionally, at least one liquid or solid carrier.

63. A pharmaceutical composition comprising at least one compound of formula I of claim 54 and/or at least one pharmaceutically acceptable salt thereof and, optionally, at least one pharmaceutically acceptable carrier.

64. A method of treating cancer comprising, administering to a warm-blooded vertebrate at least one compound of formula I of claim 54 or a pharmaceutically acceptable salt thereof.
